(19) United States
(12) Patent Application Publication
(10) Pub. No.: US 2022/0006023 A1
(43) Pub. Date: Jan. 6, 2022

(54) ORGANIC LIGHT EMITTING DIODE AND ORGANIC LIGHT EMITTING DEVICE INCLUDING THE SAME

(71) Applicant: LG DISPLAY CO., LTD., Seoul (KR)

(72) Inventors: Tae-Ryang HONG, Paju-si (KR); Gi-Hwan LIM, Paju-si (KR); Jun-Yun KIM, Paju-si (KR)

(73) Assignee: LG DISPLAY CO., LTD., Seoul (KR)

(21) Appl. No.: 17/363,744

(22) Filed: Jun. 30, 2021

(30) Foreign Application Priority Data
Jul. 1, 2020 (KR) 10-2020-0080800

(51) Int. Cl. H01L 51/00; C07D 487/04; C09K 11/06

(52) U.S. Cl. CPC H01L 51/0067; C07D 487/04; H01L 51/5012; H01L 51/0072; H01L 51/0054; C09K 11/06

(57) ABSTRACT

An organic light emitting diode (OLED) including at least one emitting material layer (EML) disposed between two electrodes and comprising a first compound and a second compound and an organic light emitting device having the OLED are disclosed. The first compound of delayed fluorescent material and the second compound of fluorescent material have predetermined energy levels so that the OLED can improve its stability and luminous lifespan.

### CROSS-REFERENCE TO RELATED APPLICATIONS

This application claims the benefit under 35 U.S.C. § 119(a) to Korean Patent Application No. 10-2020-0080800, filed in the Republic of Korea on Jul. 1, 2020, the entire contents of which are incorporated herein by reference into the present application.

### BACKGROUND

### Technical Field

The present disclosure relates to an organic light emitting diode, and more specifically, to an organic light emitting diode having excellent luminous properties and an organic light emitting device having the diode.

### Discussion of the Related Art

As display devices have become larger, there exists a need for a flat display device with lower spacing occupation. Among the flat display devices, a display device using an organic light emitting diode (OLED) has come into the spotlight, and technology relating to the OLED has been rapidly developing.

The OLED can be formed on a flexible transparent substrate such as a plastic substrate, and the OLED can be driven at a lower voltage of 10 V or less. Besides, the OLED has relatively lower power consumption for driving and the color purity of the OLED is very high. Particularly, the OLED can implement red, green and blue colors, thus it has been rapidly emerging as next-generation display device after liquid crystal display (LCD) devices, and the OLED is actually replacing the LCD in mobile display devices.

In the OLED, when electrical charges are injected into an emitting material layer between an electron injection electrode (i.e., cathode) and a hole injection electrode (i.e., anode), electrical charges are recombined to form excitons, and then emit light as the recombined excitons are shifted to a stable ground state. Conventional fluorescent materials have shown low luminous efficiency because only the singlet excitons are involved in the luminescence process thereof. The phosphorescent materials in which triplet excitons as well as the singlet excitons are involved in the luminescence process have relatively high luminous efficiency compared to the fluorescent material. However, the metal complex as the representative phosphorescent material has too short luminous lifetime to be applicable into commercial devices.

### SUMMARY

Accordingly, embodiments of the present disclosure are directed to an OLED and an organic light emitting device including the OLED that substantially obviates one or more of the problems due to the limitations and disadvantages of the related art.

An aspect of the present disclosure is to provide an OLED that can solve its low luminous lifespan and an organic light emitting device including the diode.

Another aspect of the present disclosure is to provide an OLED that can improve emission distribution in an emissive layer so as to enhance its luminous lifespan and an organic light emitting device including the diode.

Additional features and aspects will be set forth in the description that follows, and in part will be apparent from the description, or may be learned by practice of the inventive concepts provided herein. Other features and aspects of the inventive concepts may be realized and attained by the structure particularly pointed out in the written description, or derivable therefrom, and the claims hereof as well as the appended drawings.

To achieve these and other aspects of the inventive concept, as embodied and broadly described, an organic light emitting diode comprises: a first electrode; a second electrode facing the first electrode; and at least one emitting material layer disposed between the first and second electrodes, and wherein the at least one emitting material layer comprises a first compound and a second compound, wherein the first compound has the following structure of Formula 1 and the second compound has the following structure of Formula 4:

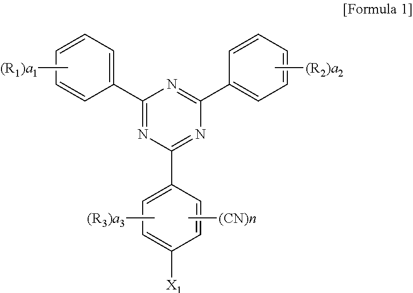

wherein each of R₁ to R₃ is independently selected from the group consisting of deuterium, tritium, a C₁-C₂₀ alkyl group, a C₆-C₃₀ aryl group and a C₃-C₄₀ hetero aryl group, or adjacent two of R₁ to R₃ form a fused ring; each of a₁ and a₂ is independently an integer of 0 to 5; a₃ is an integer of 0 to 3; n is an integer of 1 to 4; and X₁ is an unsubstituted or substituted hetero aromatic ring including at least one of N, O and S as a nuclear atom;

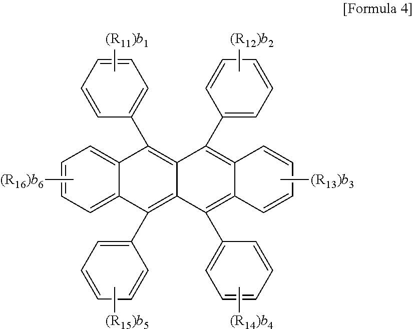

wherein each of R₁₁ to R₁₆ is independently selected from the group consisting of deuterium, tritium, a C₁-C₂₀ alkyl group, a C₆-C₃₀ aryl group and a C₃-C₄₀ hetero aryl group, or adjacent two of R₁₁ to R₁₆ form a fused ring; each of b₁, b₂, b₄ and b₅ is independently an integer of 0 to 5; and each of b₃ and b₆ is independently an integer of 0 to 4.

The first compound may have the following structure of Formula 2:

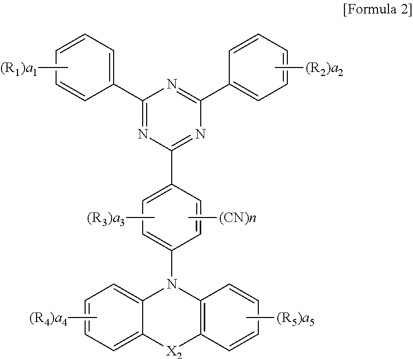

wherein each of R₁ to R₅ is independently selected from the group consisting of deuterium, tritium, a C₁-C₂₀ alkyl group, a C₆-C₃₀ aryl group and a C₃-C₄₀ hetero aryl group, or adjacent two of R₁ to R₅ form a fused ring which is unsubstituted or substituted with deuterium, tritium, a C₁-C₂₀ alkyl group, a C₆-C₃₀ aryl group or a C₃-C₄₀ hetero aryl group; each of a₁ and a₂ is independently an integer of 0 to 5; each of a₄ and as is independently an integer of 0 to 4; a₃ is an integer of 0 to 3; n is 1 an integer of 1 to 4; and X₂ is a single bond, CR₆R₇, NR₆, O or S, wherein each of R₆ and R₇ is independently selected from the group consisting of protium, deuterium, tritium, a C₁-C₂₀ alkyl group, a C₆-C₃₀ aryl group and a C₃-C₄₀ hetero aryl group.

A lowest unoccupied molecular orbital energy level (LUMO1) of the first compound may be lower than or identical to a lowest unoccupied molecular orbital energy level (LUMO2) of the second compound.

The first compound may have an energy level bandgap between about 2.0 eV and about 2.6 eV.

For example, X₁ in Formula 1 may include a carbazolyl moiety.

Contents of the first compound may be larger than contents of the second compound in the at least one emitting material layer.

In one exemplary aspect, the at least one emitting material layer has a mono-layered structure.

Alternatively, the emitting material layer may include multiple emitting material layers, and each of the first compound and the second compound is included in different emitting material layer, respectively.

In another aspect, an organic light emitting device comprises a substrate and the OLEDs disposed over the substrate, as described above.

It is to be understood that both the foregoing general description and the following detailed description are exemplary and explanatory and are intended to provide further explanation of the inventive concepts as claimed.

### BRIEF DESCRIPTION OF THE DRAWING

The accompanying drawings, which are included to provide a further understanding of the disclosure, are incorporated in and constitute a part of this application, illustrate embodiments of the disclosure and together with the description serve to explain principles of the disclosure.

FIG. 1 is a schematic cross-sectional view illustrating an organic light emitting display device as an example of an organic light emitting device in accordance with an exemplary aspect of the present disclosure.

FIG. 2 is a schematic cross-sectional view illustrating an OLED in accordance with an exemplary aspect of the present disclosure.

FIG. 3 is a schematic diagram illustrating an energy level relationship between the first compound and the second compound in an emitting material layer in accordance with the present disclosure.

FIG. 4 is a schematic diagram illustrating an energy level relationship between the first compound and the second compound in an emitting material layer in accordance with the Comparative Example.

FIG. 5 is a schematic diagram illustrating emission distribution with regard to an energy level relationship between the first compound and the second compound in accordance with the present disclosure.

FIG. 6 is a schematic diagram illustrating an emitting area in the EML for measuring an emission distribution in FIG. 5.

FIG. 7 is a schematic diagram illustrating a luminous mechanism among the luminous materials in an EML in accordance with an exemplary aspect of the present disclosure.

FIG. 8 is a schematic cross-sectional view illustrating an OLED in accordance with another exemplary aspect of the present disclosure.

FIG. 9 is a schematic cross-sectional view illustrating an OLED in accordance with still another exemplary aspect of the present disclosure.

### DETAILED DESCRIPTION

Reference and discussions will now be made below in detail to aspects, embodiments and examples of the disclosure, some examples of which are illustrated in the accompanying drawings.

The present disclosure relates to an organic light emitting diode (OLED) into which delayed fluorescent material and fluorescent material having adjusted energy levels are applied in an identical EML or adjacently disposed EMLs and an organic light emitting device having the OLED. The OLED may be applied into an organic light emitting device such as an organic light emitting display device and an organic light emitting luminescent device. As an example, a display device applying the OLED will be described.

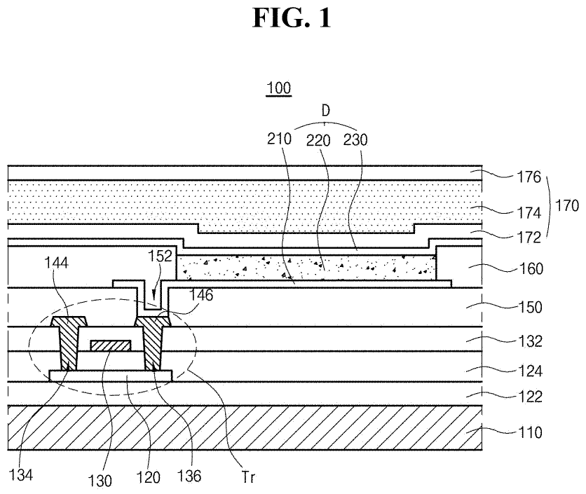

FIG. 1 is a schematic cross-sectional view of an organic light emitting display device 100 in accordance with an exemplary aspect of the present disclosure. All components of the organic light emitting device in accordance with all aspects of the present disclosure are operatively coupled and configured. As illustrated in FIG. 1, the organic light emitting display device 100 includes a substrate 110, a thin-film transistor Tr on the substrate 110, and an organic light emitting diode (OLED) D connected to the thin film transistor Tr.

The substrate 110 may include, but is not limited to, glass or plastics. For example, the substrate 110 may be made of polyimide (PI). The substrate 110, over which the thin film transistor Tr and the OLED D are arranged, form an array substrate. A buffer layer 122 may be disposed over the substrate 110, and the thin film transistor Tr is disposed over the buffer layer 122. The buffer layer 122 may be omitted.

A semiconductor layer 120 is disposed over the buffer layer 122. In one exemplary aspect, the semiconductor layer 120 may include, but is not limited to, oxide semiconductor materials. In this case, a light-shield pattern may be disposed under the semiconductor layer 120, and the light-shield pattern can prevent light from being incident toward the semiconductor layer 120, and thereby, preventing the semiconductor layer 120 from being deteriorated by the light. Alternatively, the semiconductor layer 120 may include, but is not limited to, polycrystalline silicon. In this case, opposite edges of the semiconductor layer 120 may be doped with impurities.

A gate insulating layer 124 formed of an insulating material is disposed on the semiconductor layer 120. The gate insulating layer 124 may include, but is not limited to, an inorganic insulating material such as silicon oxide (SiOₓ) or silicon nitride (SiNₓ).

A gate electrode 130 made of a conductive material such as a metal is disposed over the gate insulating layer 124 so as to correspond to a center of the semiconductor layer 120. While the gate insulating layer 124 is disposed over a whole area of the substrate 110 in FIG. 1, the gate insulating layer 124 may be patterned identically as the gate electrode 130.

An interlayer insulating layer 132 formed of an insulating material is disposed on the gate electrode 130. The interlayer insulating layer 132 may include, but is not limited to, an inorganic insulating material such as silicon oxide (SiOₓ) or silicon nitride (SiNₓ), or an organic insulating material such as benzocyclobutene or photo-acryl.

The interlayer insulating layer 132 has first and second semiconductor layer contact holes 134 and 136 that expose both sides of the semiconductor layer 120. The first and second semiconductor layer contact holes 134 and 136 are disposed over opposite sides of the gate electrode 130 with spacing apart from the gate electrode 130. The first and second semiconductor layer contact holes 134 and 136 are formed within the gate insulating layer 124 in FIG. 1. Alternatively, the first and second semiconductor layer contact holes 134 and 136 are formed only within the interlayer insulating layer 132 when the gate insulating layer 124 is patterned identically as the gate electrode 130.

A source electrode 144 and a drain electrode 146, which are formed of conductive material such as a metal, are disposed on the interlayer insulating layer 132. The source electrode 144 and the drain electrode 146 are spaced apart from each other with respect to the gate electrode 130, and contact both sides of the semiconductor layer 120 through the first and second semiconductor layer contact holes 134 and 136, respectively.

The semiconductor layer 120, the gate electrode 130, the source electrode 144 and the drain electrode 146 constitute the thin film transistor Tr, which acts as a driving element. The thin film transistor Tr in FIG. 1 has a coplanar structure in which the gate electrode 130, the source electrode 144 and the drain electrode 146 are disposed over the semiconductor layer 120. Alternatively, the thin film transistor Tr may have an inverted staggered structure in which a gate electrode is disposed under a semiconductor layer and a source and drain electrodes are disposed over the semiconductor layer. In this case, the semiconductor layer may comprise amorphous silicon.

A gate line and a data line, which cross each other to define a pixel region, and a switching element, which is connected to the gate line and the data line, may be further formed in the pixel region of FIG. 1. The switching element is connected to the thin film transistor Tr, which is a driving element. Besides, a power line is spaced apart in parallel from the gate line or the data line, and the thin film transistor Tr may further include a storage capacitor configured to constantly keep a voltage of the gate electrode for one frame.

A passivation layer 150 is disposed on the source and drain electrodes 144 and 146 over the whole substrate 110. The passivation layer 150 has a drain contact hole 152 that exposes the drain electrode 146 of the thin film transistor Tr.

A first electrode 210 is disposed on the passivation layer 150 and is connected to the drain electrode 146 of the thin film transistor Tr through the drain contact hole 152. The first electrode 210 is disposed in each pixel region. The first electrode 210 may be an anode and include a conductive material having a relatively high work function value. For example, the first electrode 210 may include, but is not limited to, a transparent conductive material such as indium tin oxide (ITO), indium zinc oxide (IZO), indium tin zinc oxide (ITZO), tin oxide (SnO), zinc oxide (ZnO), indium cerium oxide (ICO), aluminum doped zinc oxide (AZO), and the like.

In one exemplary aspect, when the organic light emitting display device 100 is a top-emission type, a reflective electrode or a reflective layer may be disposed under the first electrode 210. For example, the reflective electrode or the reflective layer may include, but are not limited to, silver (Ag) or aluminum-palladium-copper (APC) alloy.

A bank layer 160 is disposed on the passivation layer 150 in order to cover edges of the first electrode 210. The bank layer 160 corresponds to pixel region and exposes a center of the first electrode 210.

An emissive layer 220 is disposed on the first electrode 210. In one exemplary aspect, the emissive layer 220 may have a mono-layered structure of an emitting material layer (EML). Alternatively, the emissive layer 220 may have a multiple-layered structure in order to enhance its luminous efficiency. For example, the emissive layer 220 may comprise an EML 240 disposed between the first and second electrodes 210 and 230, and may further comprise a hole transport layer (HTL) 260 disposed between the first electrode 210 and the EML 240 and/or an electron transport layer (ETL) 270 disposed between the second electrode 230 and the EML 240. In addition, the emissive layer 220 may further comprise a hole injection layer (HIL) 250 disposed between the first electrode 210 and the HTL 260 and/or an electron injection layer (EIL) 280 disposed between the second electrode 230 and the ETL 270. Alternatively, the emissive layer 220 may further comprise an electron blocking layer (EBL) 265 disposed between the HTL 260 and the EML 240 and/or a hole blocking layer (HBL) disposed between the EML 240 and the ETL 270.

The second electrode 230 is disposed over the substrate 110 above which the emissive layer 220 is disposed. The second electrode 230 may be disposed over a whole display area and may include a conductive material with a relatively low work function value compared to the first electrode 210. The second electrode 230 may be a cathode. For example, the second electrode 230 may include, but is not limited to, aluminum (Al), magnesium (Mg), calcium (Ca), silver (Ag), alloy thereof or combination thereof such as aluminum-magnesium alloy (Al—Mg). The first electrode 210, the emissive layer 220 and the second electrode 230 constitutes the OLED D.

In addition, an encapsulation film 170 may be disposed over the second electrode 230 in order to prevent outer moisture from penetrating into the OLED D. The encapsulation film 170 may have, but is not limited to, a laminated structure of a first inorganic insulating film 172, an organic insulating film 174 and a second inorganic insulating film 176.

Moreover, the organic light emitting display device 100 may have a polarizer in order to decrease external light reflection. For example, the polarizer may be a circular polarizer. In addition, a cover window may be attached to the encapsulation film 170 or the polarizer. In this case, the substrate 110 and the cover window may have a flexible property, thus the organic light emitting display device 100 may be a flexible display device.

In addition, the organic light emitting display device 100 may include a color filter layer that comprises dyes or pigments for transmitting specific wavelength light of light emitted from the OLED D. For example, the color filter layer can transmit light of specific wavelength such as red (R), green (G), blue (B) and/or white (W). Each of red, green, and blue color filter may be formed separately in each pixel region and may be disposed correspondingly to the emissive layer 230 of the OLED D emitting light, respectively so that the organic light emitting display device 100 can implement full-color through the color filter.

For example, when the organic light emitting display device 100 is a bottom-emission type, the color filter layer may be disposed on the interlayer insulating layer 132 with corresponding to the OLED D. Alternatively, when the organic light emitting display device 100 is a top-emission type, the color filter layer may be disposed over the OLED D, that is, a second electrode 230.

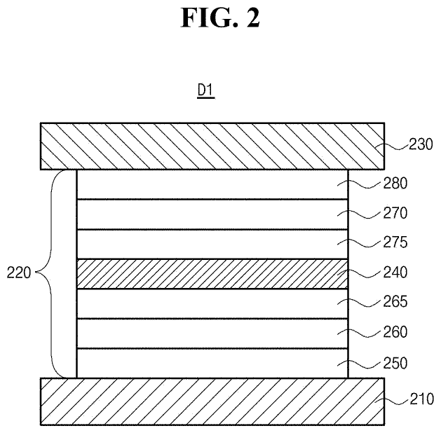

FIG. 2 is a schematic cross-sectional view illustrating an OLED in accordance with an exemplary aspect of the present disclosure. In this aspect, the EML 240 having a mono-layered structure includes a first compound (Compound 1) DF of delayed fluorescent material and a second compound (Compound 2) FD of fluorescent material, and optionally a third compound (Compound 3) H of host.

General fluorescent material emits light when excitons at singlet state S₁ drops to ground state S₀. Since the singlet excitons and triplet excitons are generated in a ratio of 1:3, the general fluorescent material has maximum 25% of internal quantum efficiency in theory.

On the other hand, delayed fluorescent material can activate the triplet excitons by heat or electrical field in driving the OLED so that the triplet excitons in the delayed fluorescent material can be involved in luminous process. The activated triplet excitons in delayed fluorescent material can be converted to its own singlet state, and then the delayed fluorescent material emits light as the singlet excitons drops to the ground state. All excitons in the delayed fluorescent material can be involved in the luminous process, and therefore the delayed fluorescent material can implement maximally 100% of internal quantum efficiency in theory.

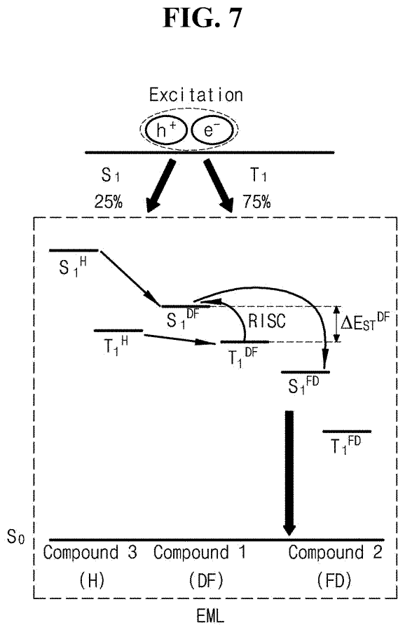

The delayed fluorescent material must have narrow energy level bandgap ΔE_{ST}^{DF} equal to or less than about 0.3 eV, for example, between about 0.05 and about 0.3 eV, between a singlet energy level S₁^{DF} and an triplet energy level T₁^{DF} (see, FIG. 7). The material having small energy level bandgap between the singlet state and the triplet state can exhibit common fluorescence with Inter System Crossing (ISC) in which the exciton energy of singlet state can be transferred to exciton energy of triplet state, as well as delayed fluorescence with Reverser Inter System Crossing (RISC) in which the exciton energy of triplet state can be converted upwardly to the exciton energy of singlet state, and then the excitons of singlet energy level can be transferred to the ground state S₀^{DF}. The delayed fluorescent material may have 100% of internal quantum efficiency in theory as the conventional phosphorescent materials including heavy metal.

The first compound DF of the delayed fluorescent material in the EML 240 may has the following structure of Formula 1:

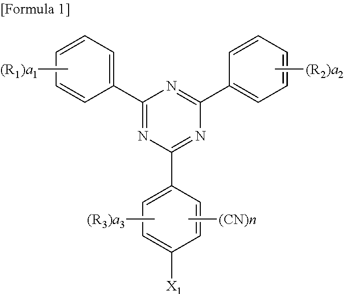

In Formula 1, each of R₁ to R₃ is independently selected from the group consisting of deuterium, tritium, a C₁-C₂₀ alkyl group, a C₆-C₃₀ aryl group and a C₃-C₄₀ hetero aryl group, or adjacent two of R₁ to R₃ form a fused ring; each of a₁ and a₂ is independently an integer of 0 to 5; a₃ is an integer of 0 to 3; n is an integer of 1 to 4; and X₁ is an unsubstituted or substituted hetero aromatic ring including at least one of N, O and S as a nuclear atom. The fused ring may be a C₆-C₂₀ fused alicyclic ring, a C₄-C₂₀ fused hetero alicyclic ring, a C₆-C₂₀ fused aromatic ring or a C₄-C₂₀ fused hetero aromatic ring.

Each of the alkyl group, the aryl group and the hetero aryl group may be independently substituted or unsubstituted. In addition, n may be an integer of 1 or 2 and X₁ may be a hetero aromatic ring including at least two of N, O and S or at least two Ns as a nuclear atom.

As an example, the first compound DF may have the following structure of Formula 2:

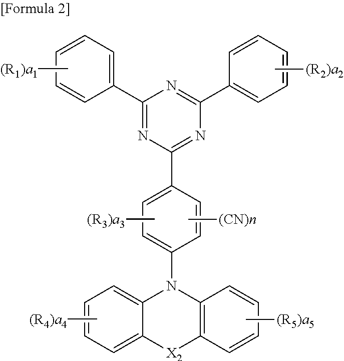

In Formula 2, each of R₁ to R₅ is independently selected from the group consisting of deuterium, tritium, a C₁-C₂₀ alkyl group, a C₆-C₃₀ aryl group and a C₃-C₄₀ hetero aryl group, or adjacent two of R₁ to R₅ form a fused ring which is unsubstituted or substituted with deuterium, tritium, a C₁-C₂₀ alkyl group, a C₆-C₃₀ aryl group or a C₃-C₄₀ hetero aryl group; each of a₁ and a₂ is independently an integer of 0 to 5; each of a₄ and as is independently an integer of 0 to 4; a₃ is an integer of 0 to 3; n is an integer of 1 to 4; and X₂ is a single bond, CR₆R₇, NR₆, O or S, wherein each of R₆ and R₇ is independently selected from the group consisting of protium, deuterium, tritium, a C₁-C₂₀ alkyl group, a C₆-C₃₀ aryl group and a C₃-C₄₀ hetero aryl group.

For example, n may be an integer of 1 or 2 and each of R₄ and R₅ may be a C₁₂-Cis polycyclic hetero aryl group having 0, N and/or S as a nuclear atom in Formula 2.

In an exemplary embodiment, X₁ in Formula 1 may include a carbazolyl moiety. The carbazolyl moiety may include, but is not limited to, an indeno-carbazolyl moiety, an indolo-carbazolyl moiety, a benzothieno-carbazolyl moiety and a benzofuro-carbazolyl moiety, each of which may be independently unsubstituted or substituted with at least one of a C₁-C₂₀ alkyl group, a C₆-C₃₀ aryl group and a C₄-C₃₀ hetero aryl group.

More particular, the first compound DF may be selected from the following compounds having the structure of Formula 3:

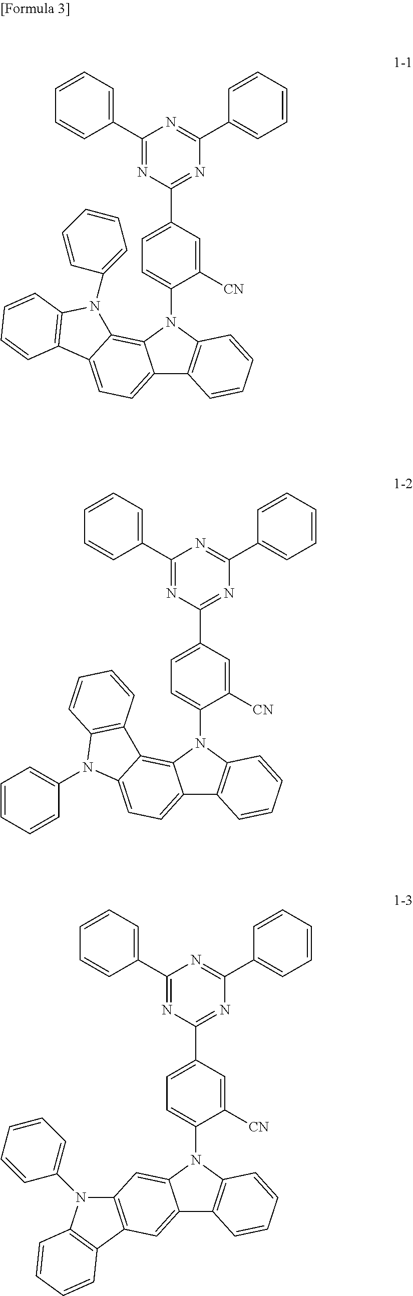 [SMILES: N#Cc1cc(-c2nc(-c3ccccc3)nc(-c3ccccc3)n2)ccc1-n1c2ccccc2c2ccc3c4ccccc4n(-c4ccccc4)c3c21 ; N#Cc1cc(-c2nc(-c3ccccc3)nc(-c3ccccc3)n2)ccc1-n1c2ccccc2c2ccc3c(c4ccccc4n3-c3ccccc3)c21 ; N#Cc1cc(-c2nc(-c3ccccc3)nc(-c3ccccc3)n2)ccc1-n1c2ccccc2c2cc3c4ccccc4n(-c4ccccc4)c3cc21]

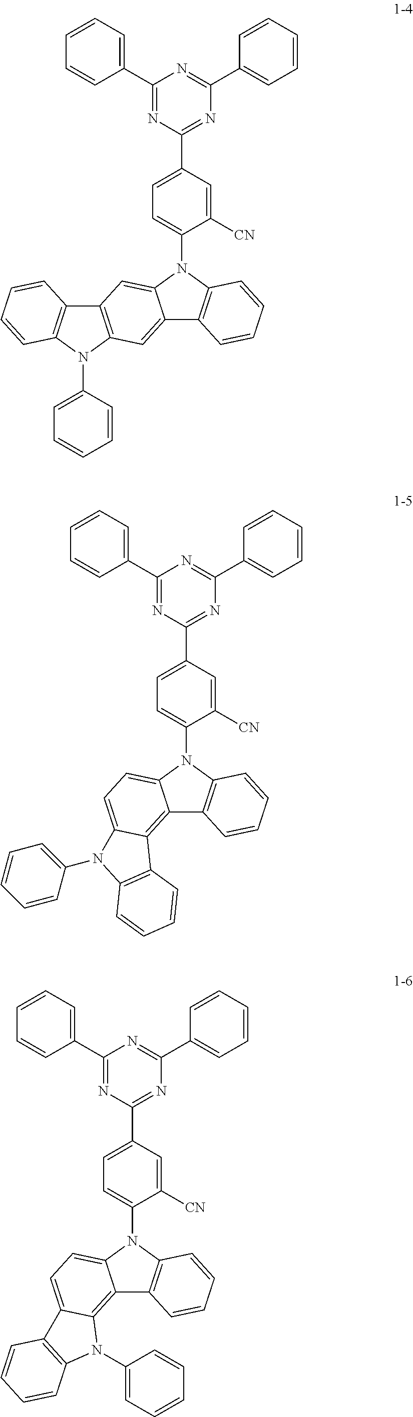 [SMILES: N#Cc1cc(-c2nc(-c3ccccc3)nc(-c3ccccc3)n2)ccc1-n1c2ccccc2c2cc3c(cc21)c1ccccc1n3-c1ccccc1 ; N#Cc1cc(-c2nc(-c3ccccc3)nc(-c3ccccc3)n2)ccc1-n1c2ccccc2c2c3c4ccccc4n(-c4ccccc4)c3ccc21 ; N#Cc1cc(-c2nc(-c3ccccc3)nc(-c3ccccc3)n2)ccc1-n1c2ccccc2c2c1ccc1c3ccccc3n(-c3ccccc3)c12]

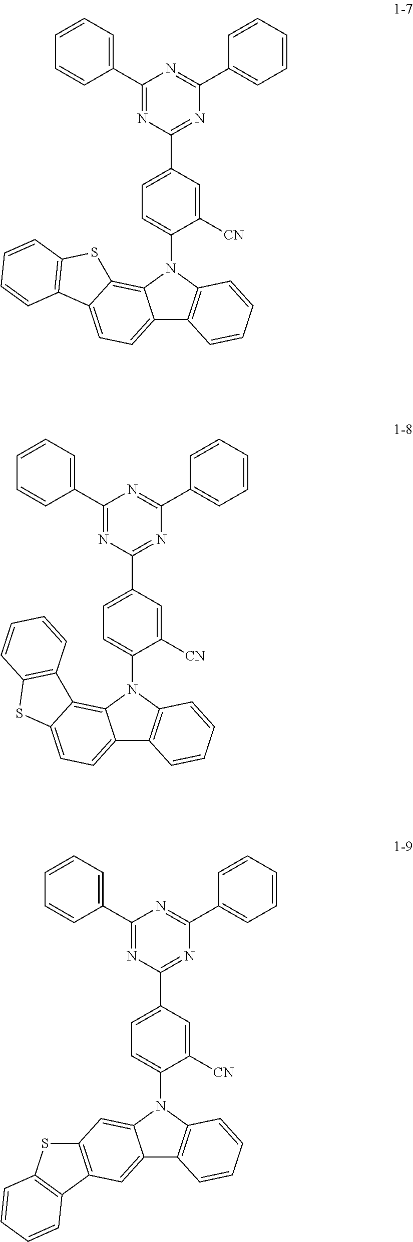 [SMILES: N#Cc1cc(-c2nc(-c3ccccc3)nc(-c3ccccc3)n2)ccc1-n1c2ccccc2c2ccc3c4ccccc4sc3c21 ; N#Cc1cc(-c2nc(-c3ccccc3)nc(-c3ccccc3)n2)ccc1-n1c2ccccc2c2ccc3sc4ccccc4c3c21 ; N#Cc1cc(-c2nc(-c3ccccc3)nc(-c3ccccc3)n2)ccc1-n1c2ccccc2c2cc3c(cc21)sc1ccccc13]

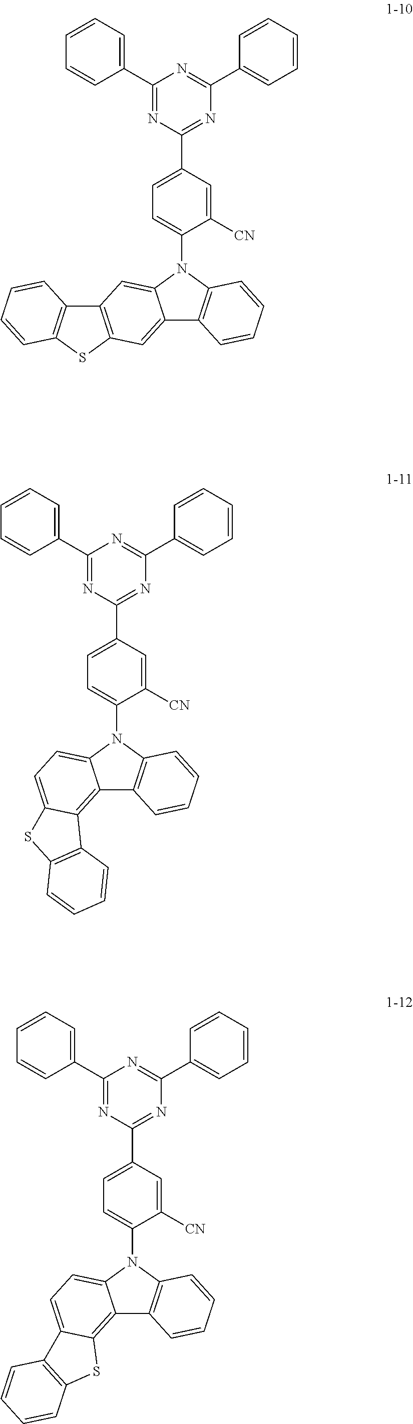 [SMILES: N#Cc1cc(-c2nc(-c3ccccc3)nc(-c3ccccc3)n2)ccc1-n1c2ccccc2c2cc3sc4ccccc4c3cc21 ; N#Cc1cc(-c2nc(-c3ccccc3)nc(-c3ccccc3)n2)ccc1-n1c2ccccc2c2c3c(ccc21)sc1ccccc13 ; N#Cc1cc(-c2nc(-c3ccccc3)nc(-c3ccccc3)n2)ccc1-n1c2ccccc2c2c3sc4ccccc4c3ccc21]

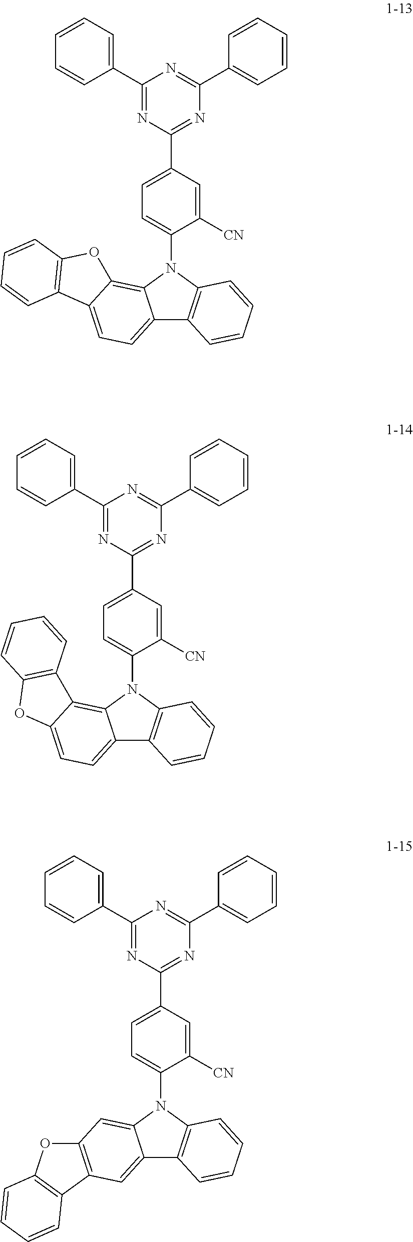 [SMILES: N#Cc1cc(-c2nc(-c3ccccc3)nc(-c3ccccc3)n2)ccc1-n1c2ccccc2c2ccc3c4ccccc4oc3c21 ; N#Cc1cc(-c2nc(-c3ccccc3)nc(-c3ccccc3)n2)ccc1-n1c2ccccc2c2ccc3oc4ccccc4c3c21 ; N#Cc1cc(-c2nc(-c3ccccc3)nc(-c3ccccc3)n2)ccc1-n1c2ccccc2c2cc3c(cc21)oc1ccccc13]

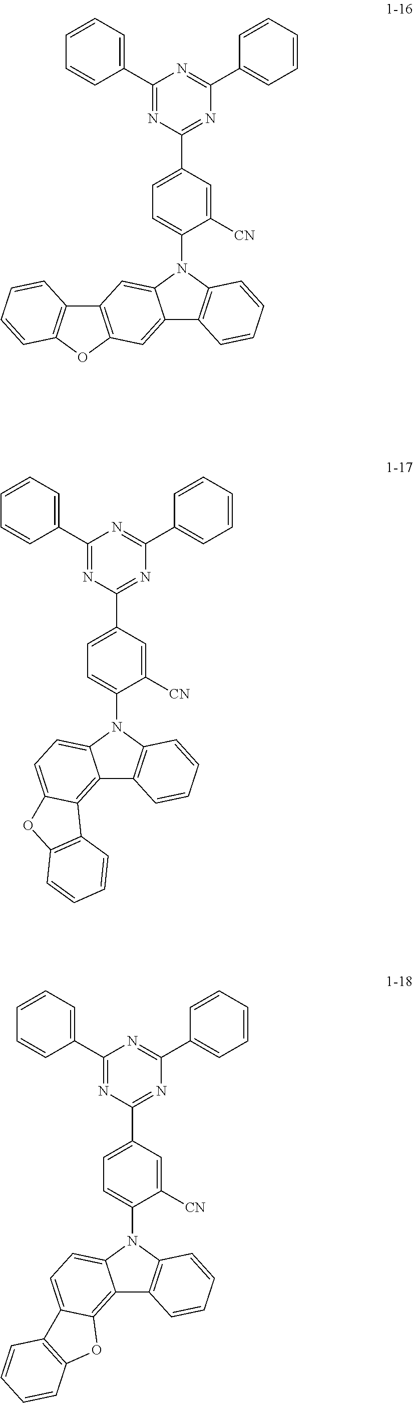 [SMILES: N#Cc1cc(-c2nc(-c3ccccc3)nc(-c3ccccc3)n2)ccc1-n1c2ccccc2c2cc3oc4ccccc4c3cc21 ; N#Cc1cc(-c2nc(-c3ccccc3)nc(-c3ccccc3)n2)ccc1-n1c2ccccc2c2c3c(ccc21)oc1ccccc13 ; N#Cc1cc(-c2nc(-c3ccccc3)nc(-c3ccccc3)n2)ccc1-n1c2ccccc2c2c3oc4ccccc4c3ccc21]

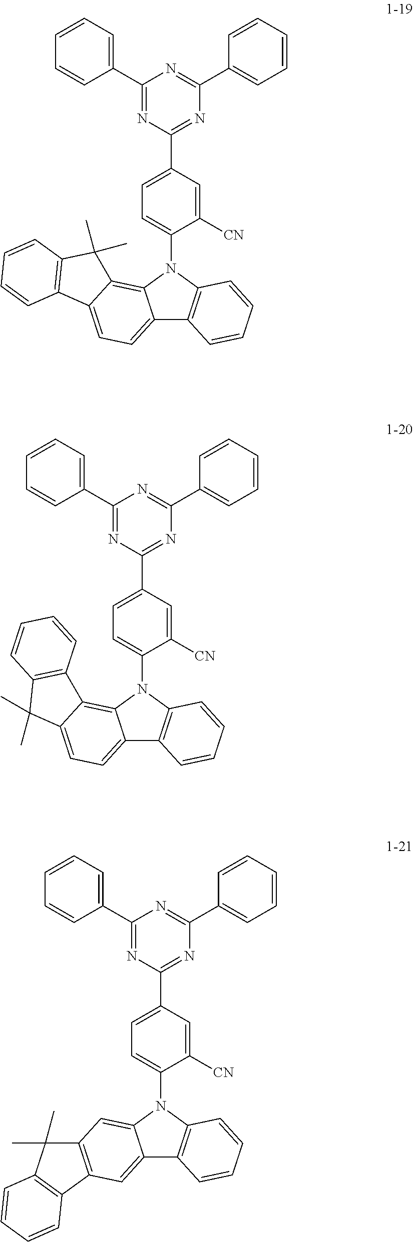 [SMILES: CC1(C)c2ccccc2-c2ccc3c4ccccc4n(-c4ccc(-c5nc(-c6ccccc6)nc(-c6ccccc6)n5)cc4C#N)c3c21 ; CC1(C)c2ccccc2-c2c1ccc1c3ccccc3n(-c3ccc(-c4nc(-c5ccccc5)nc(-c5ccccc5)n4)cc3C#N)c21 ; CC1(C)c2ccccc2-c2cc3c4ccccc4n(-c4ccc(-c5nc(-c6ccccc6)nc(-c6ccccc6)n5)cc4C#N)c3cc21]

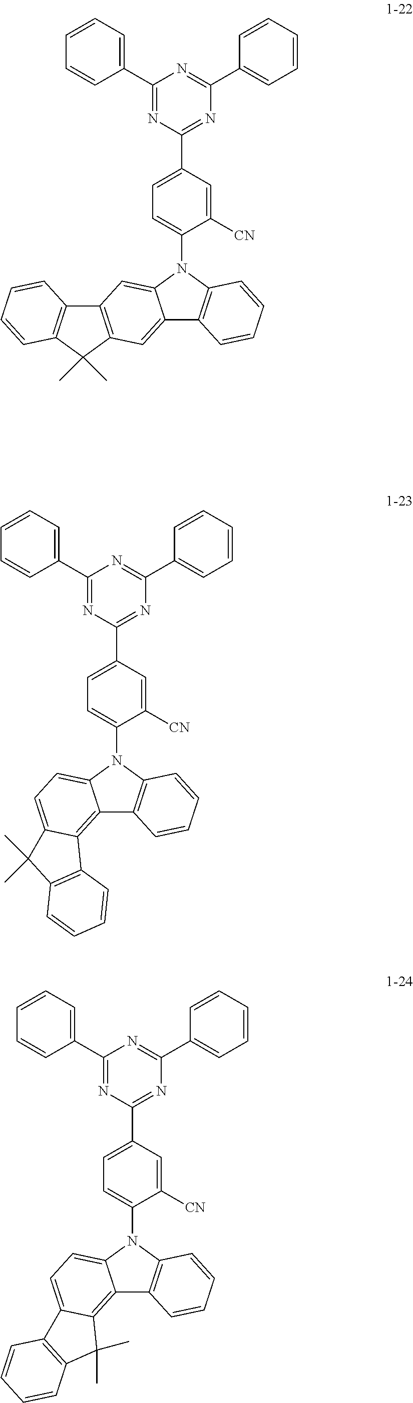 [SMILES: CC1(C)c2ccccc2-c2cc3c(cc21)c1ccccc1n3-c1ccc(-c2nc(-c3ccccc3)nc(-c3ccccc3)n2)cc1C#N ; CC1(C)c2ccccc2-c2c1ccc1c2c2ccccc2n1-c1ccc(-c2nc(-c3ccccc3)nc(-c3ccccc3)n2)cc1C#N ; CC1(C)c2ccccc2-c2ccc3c(c21)c1ccccc1n3-c1ccc(-c2nc(-c3ccccc3)nc(-c3ccccc3)n2)cc1C#N]

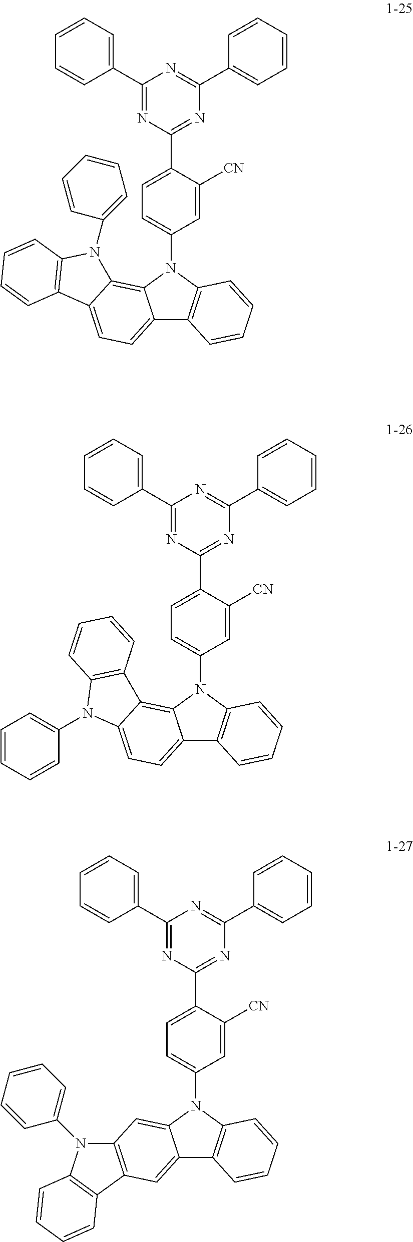 [SMILES: N#Cc1cc(-n2c3ccccc3c3ccc4c5ccccc5n(-c5ccccc5)c4c32)ccc1-c1nc(-c2ccccc2)nc(-c2ccccc2)n1 ; N#Cc1cc(-n2c3ccccc3c3ccc4c(c5ccccc5n4-c4ccccc4)c32)ccc1-c1nc(-c2ccccc2)nc(-c2ccccc2)n1 ; N#Cc1cc(-n2c3ccccc3c3cc4c5ccccc5n(-c5ccccc5)c4cc32)ccc1-c1nc(-c2ccccc2)nc(-c2ccccc2)n1]

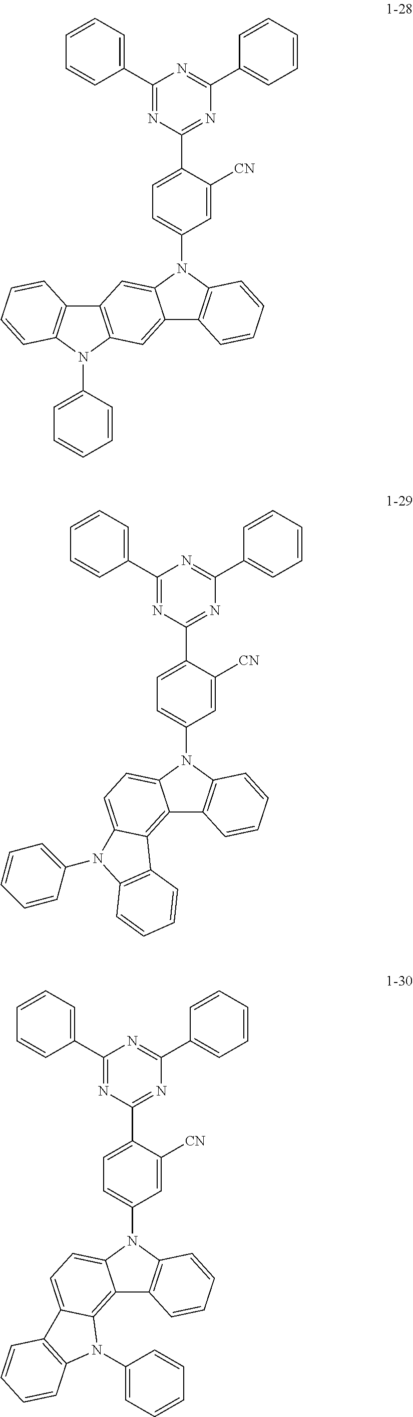 [SMILES: N#Cc1cc(-n2c3ccccc3c3cc4c(cc32)c2ccccc2n4-c2ccccc2)ccc1-c1nc(-c2ccccc2)nc(-c2ccccc2)n1 ; N#Cc1cc(-n2c3ccccc3c3c4c5ccccc5n(-c5ccccc5)c4ccc32)ccc1-c1nc(-c2ccccc2)nc(-c2ccccc2)n1 ; N#Cc1cc(-n2c3ccccc3c3c2ccc2c4ccccc4n(-c4ccccc4)c23)ccc1-c1nc(-c2ccccc2)nc(-c2ccccc2)n1]

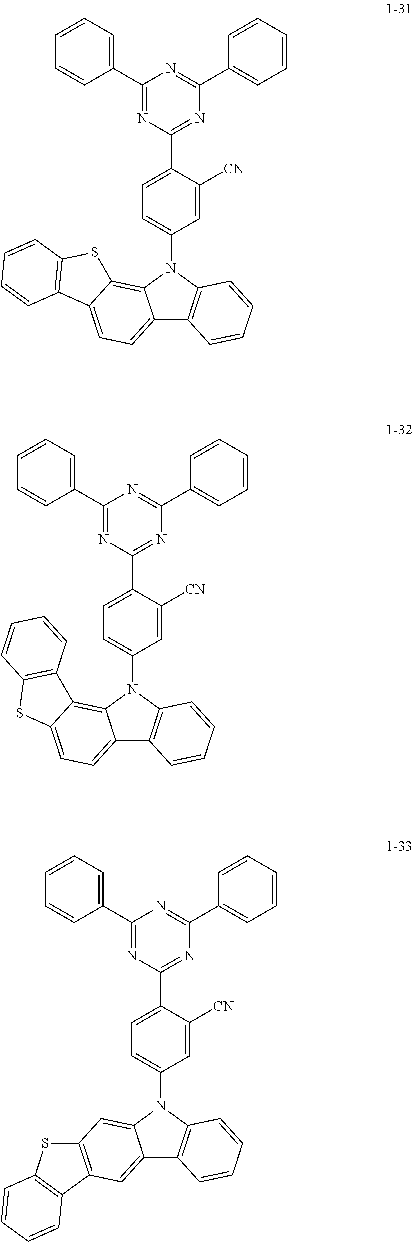 [SMILES: N#Cc1cc(-n2c3ccccc3c3ccc4c5ccccc5sc4c32)ccc1-c1nc(-c2ccccc2)nc(-c2ccccc2)n1 ; N#Cc1cc(-n2c3ccccc3c3ccc4sc5ccccc5c4c32)ccc1-c1nc(-c2ccccc2)nc(-c2ccccc2)n1 ; N#Cc1cc(-n2c3ccccc3c3cc4c(cc32)sc2ccccc24)ccc1-c1nc(-c2ccccc2)nc(-c2ccccc2)n1]

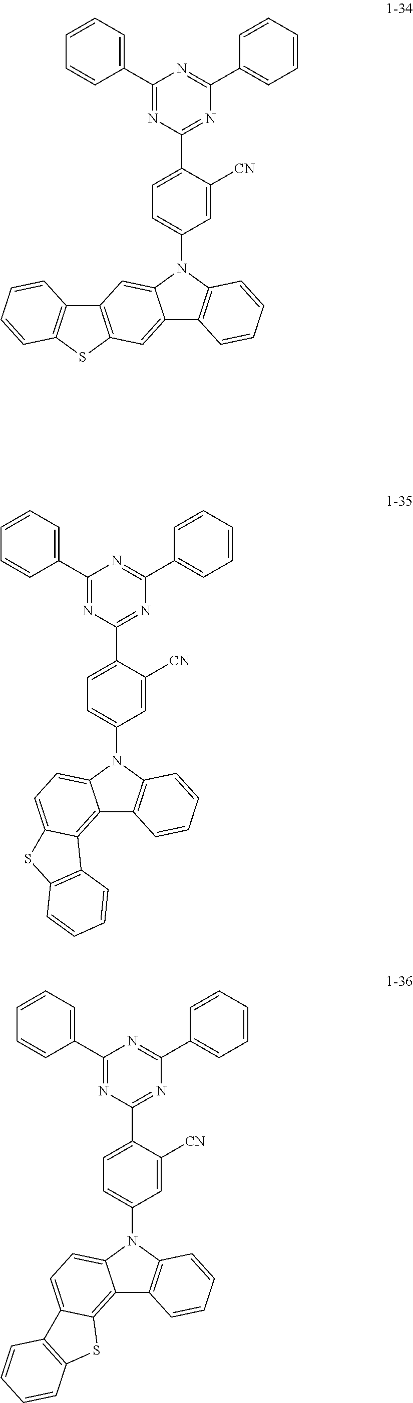 [SMILES: N#Cc1cc(-n2c3ccccc3c3cc4sc5ccccc5c4cc32)ccc1-c1nc(-c2ccccc2)nc(-c2ccccc2)n1 ; N#Cc1cc(-n2c3ccccc3c3c4c(ccc32)sc2ccccc24)ccc1-c1nc(-c2ccccc2)nc(-c2ccccc2)n1 ; N#Cc1cc(-n2c3ccccc3c3c4sc5ccccc5c4ccc32)ccc1-c1nc(-c2ccccc2)nc(-c2ccccc2)n1]

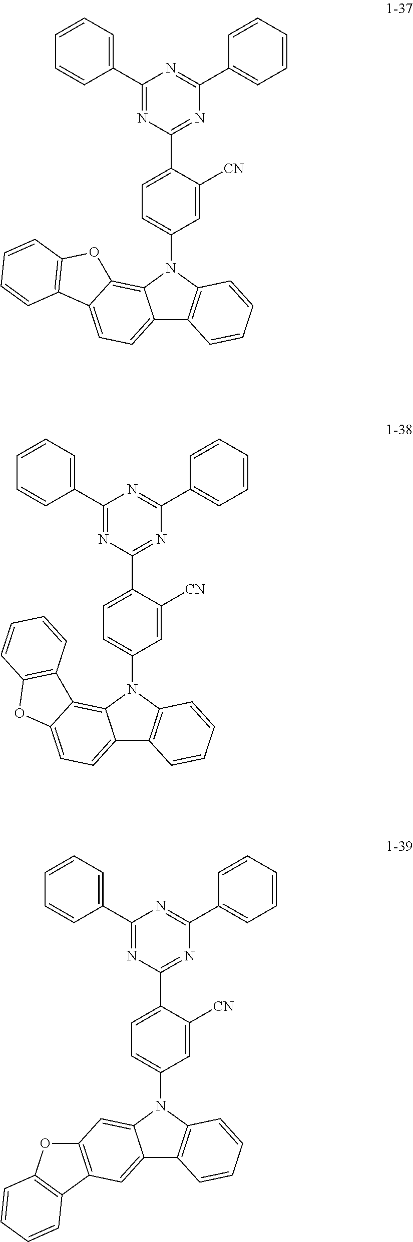 [SMILES: N#Cc1cc(-n2c3ccccc3c3ccc4c5ccccc5oc4c32)ccc1-c1nc(-c2ccccc2)nc(-c2ccccc2)n1 ; N#Cc1cc(-n2c3ccccc3c3ccc4oc5ccccc5c4c32)ccc1-c1nc(-c2ccccc2)nc(-c2ccccc2)n1 ; N#Cc1cc(-n2c3ccccc3c3cc4c(cc32)oc2ccccc24)ccc1-c1nc(-c2ccccc2)nc(-c2ccccc2)n1]

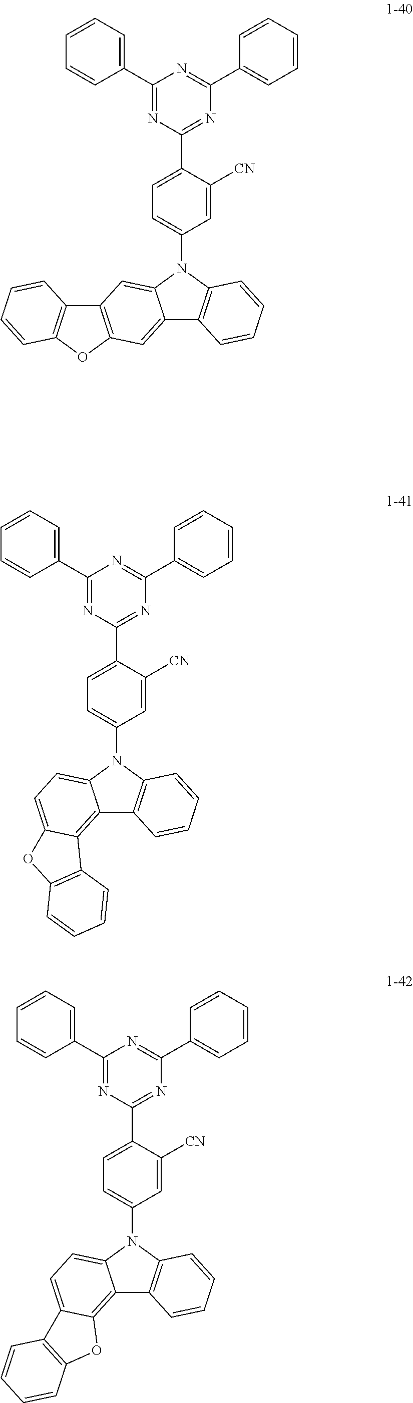 [SMILES: N#Cc1cc(-n2c3ccccc3c3cc4oc5ccccc5c4cc32)ccc1-c1nc(-c2ccccc2)nc(-c2ccccc2)n1 ; N#Cc1cc(-n2c3ccccc3c3c4c(ccc32)oc2ccccc24)ccc1-c1nc(-c2ccccc2)nc(-c2ccccc2)n1 ; N#Cc1cc(-n2c3ccccc3c3c4oc5ccccc5c4ccc32)ccc1-c1nc(-c2ccccc2)nc(-c2ccccc2)n1]

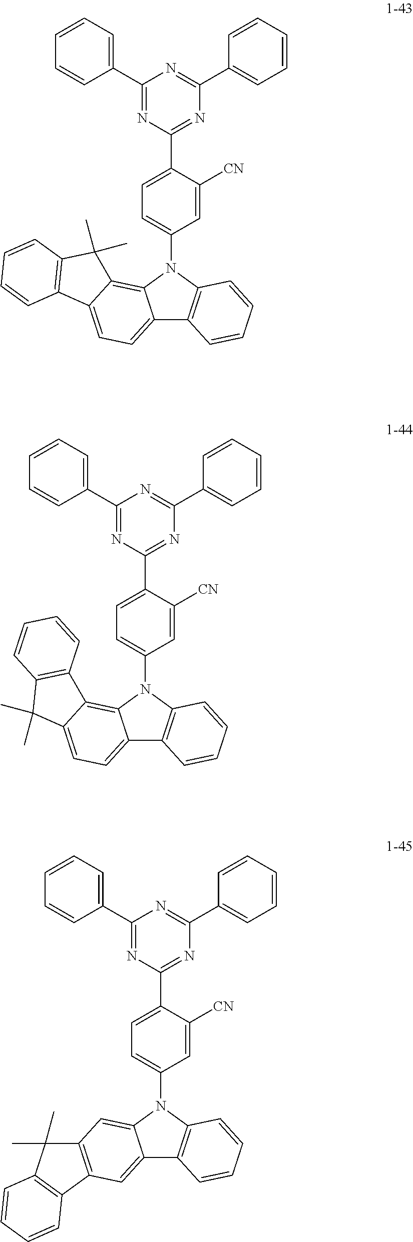 [SMILES: CC1(C)c2ccccc2-c2ccc3c4ccccc4n(-c4ccc(-c5nc(-c6ccccc6)nc(-c6ccccc6)n5)c(C#N)c4)c3c21 ; CC1(C)c2ccccc2-c2c1ccc1c3ccccc3n(-c3ccc(-c4nc(-c5ccccc5)nc(-c5ccccc5)n4)c(C#N)c3)c21 ; CC1(C)c2ccccc2-c2cc3c4ccccc4n(-c4ccc(-c5nc(-c6ccccc6)nc(-c6ccccc6)n5)c(C#N)c4)c3cc21]

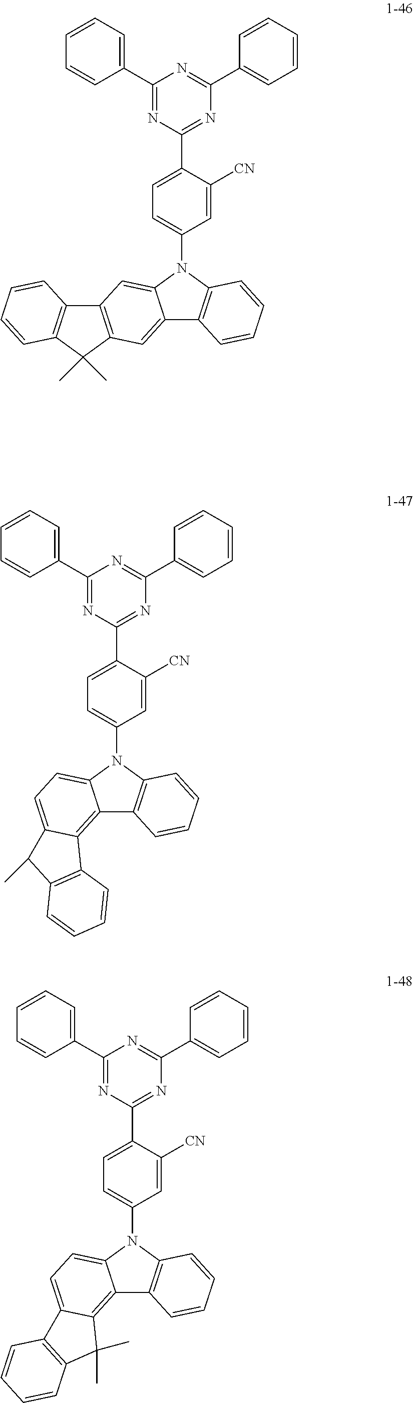 [SMILES: CC1(C)c2ccccc2-c2cc3c(cc21)c1ccccc1n3-c1ccc(-c2nc(-c3ccccc3)nc(-c3ccccc3)n2)c(C#N)c1 ; CC1c2ccccc2-c2c1ccc1c2c2ccccc2n1-c1ccc(-c2nc(-c3ccccc3)nc(-c3ccccc3)n2)c(C#N)c1 ; CC1(C)c2ccccc2-c2ccc3c(c21)c1ccccc1n3-c1ccc(-c2nc(-c3ccccc3)nc(-c3ccccc3)n2)c(C#N)c1]

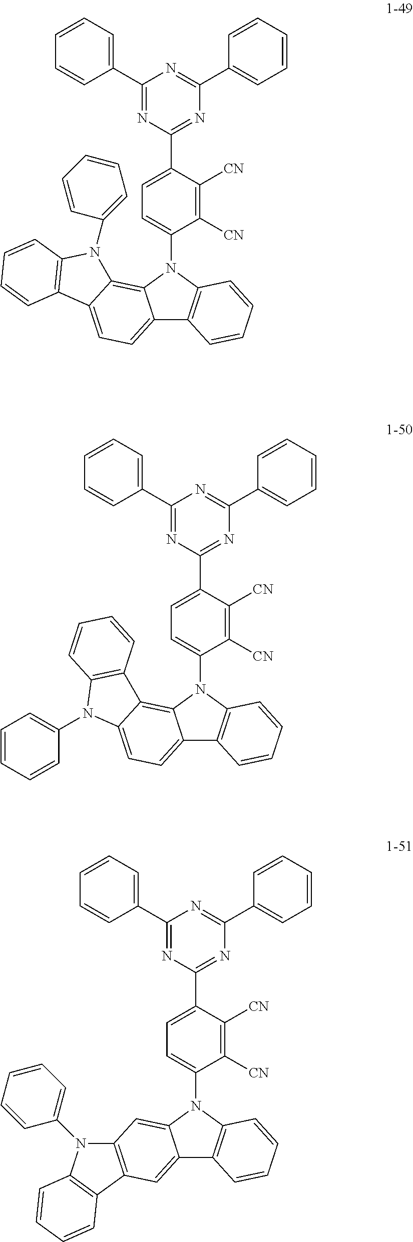 [SMILES: N#Cc1c(-c2nc(-c3ccccc3)nc(-c3ccccc3)n2)ccc(-n2c3ccccc3c3ccc4c5ccccc5n(-c5ccccc5)c4c32)c1C#N ; N#Cc1c(-c2nc(-c3ccccc3)nc(-c3ccccc3)n2)ccc(-n2c3ccccc3c3ccc4c(c5ccccc5n4-c4ccccc4)c32)c1C#N ; N#Cc1c(-c2nc(-c3ccccc3)nc(-c3ccccc3)n2)ccc(-n2c3ccccc3c3cc4c5ccccc5n(-c5ccccc5)c4cc32)c1C#N]

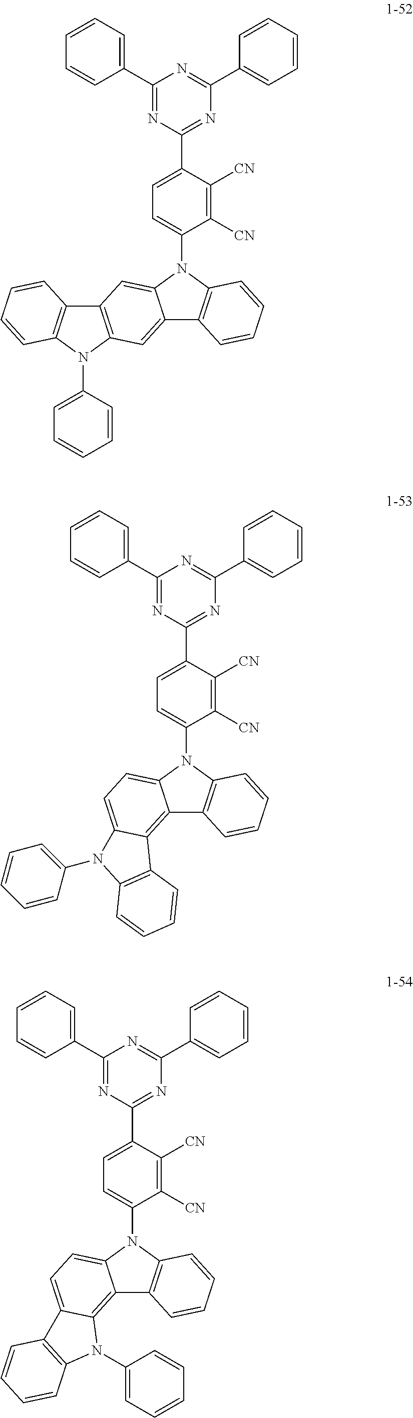 [SMILES: N#Cc1c(-c2nc(-c3ccccc3)nc(-c3ccccc3)n2)ccc(-n2c3ccccc3c3cc4c(cc32)c2ccccc2n4-c2ccccc2)c1C#N ; N#Cc1c(-c2nc(-c3ccccc3)nc(-c3ccccc3)n2)ccc(-n2c3ccccc3c3c4c5ccccc5n(-c5ccccc5)c4ccc32)c1C#N ; N#Cc1c(-c2nc(-c3ccccc3)nc(-c3ccccc3)n2)ccc(-n2c3ccccc3c3c2ccc2c4ccccc4n(-c4ccccc4)c23)c1C#N]

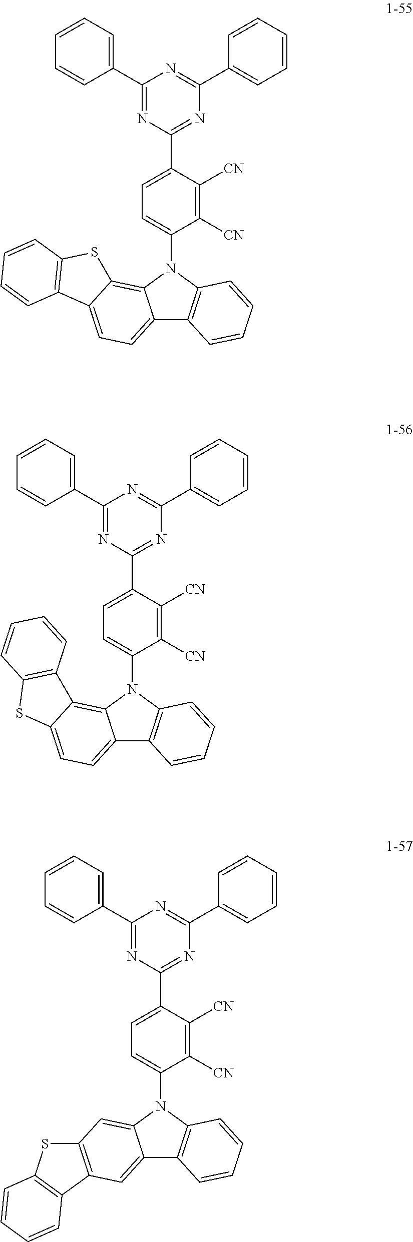 [SMILES: N#Cc1c(-c2nc(-c3ccccc3)nc(-c3ccccc3)n2)ccc(-n2c3ccccc3c3ccc4c5ccccc5sc4c32)c1C#N ; N#Cc1c(-c2nc(-c3ccccc3)nc(-c3ccccc3)n2)ccc(-n2c3ccccc3c3ccc4sc5ccccc5c4c32)c1C#N ; N#Cc1c(-c2nc(-c3ccccc3)nc(-c3ccccc3)n2)ccc(-n2c3ccccc3c3cc4c(cc32)sc2ccccc24)c1C#N]

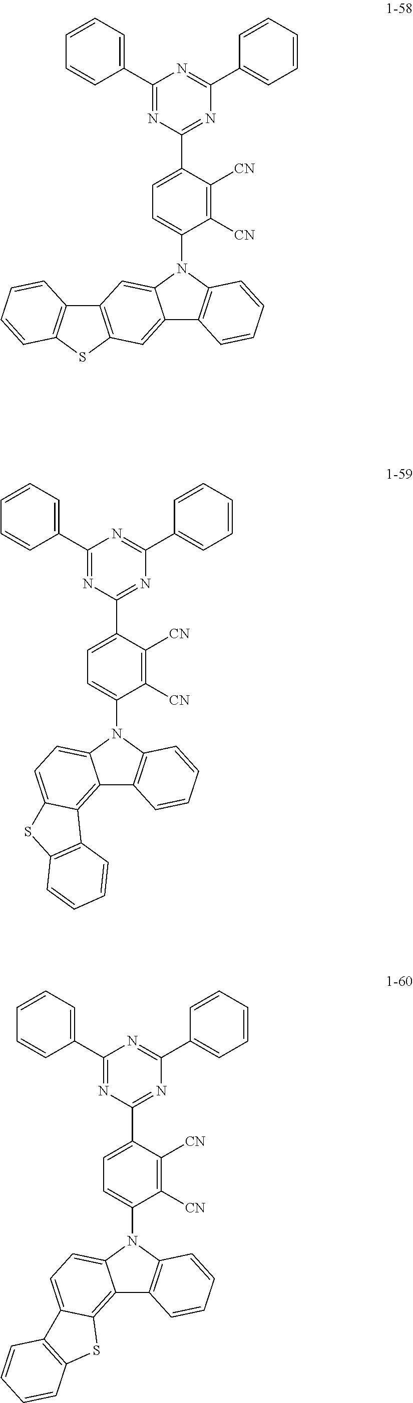 [SMILES: N#Cc1c(-c2nc(-c3ccccc3)nc(-c3ccccc3)n2)ccc(-n2c3ccccc3c3cc4sc5ccccc5c4cc32)c1C#N ; N#Cc1c(-c2nc(-c3ccccc3)nc(-c3ccccc3)n2)ccc(-n2c3ccccc3c3c4c(ccc32)sc2ccccc24)c1C#N ; N#Cc1c(-c2nc(-c3ccccc3)nc(-c3ccccc3)n2)ccc(-n2c3ccccc3c3c4sc5ccccc5c4ccc32)c1C#N]

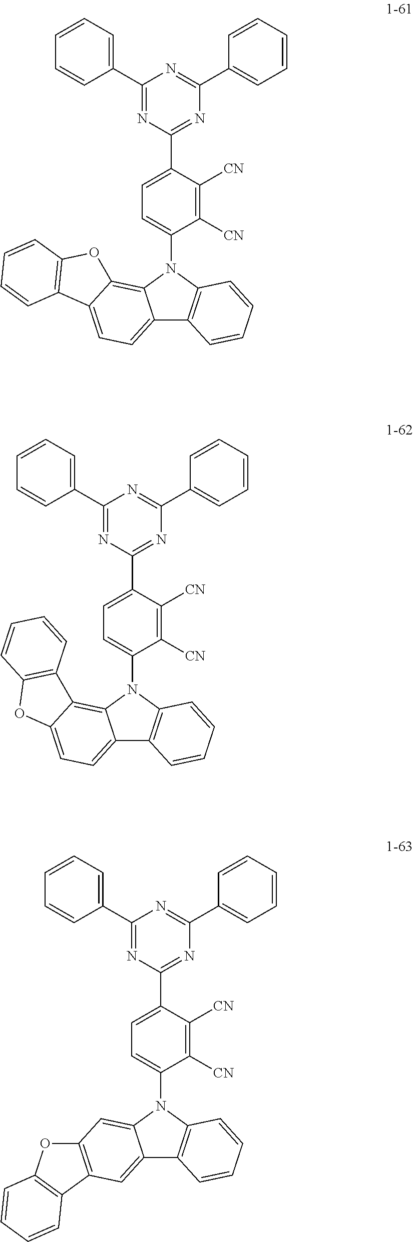 [SMILES: N#Cc1c(-c2nc(-c3ccccc3)nc(-c3ccccc3)n2)ccc(-n2c3ccccc3c3ccc4c5ccccc5oc4c32)c1C#N ; N#Cc1c(-c2nc(-c3ccccc3)nc(-c3ccccc3)n2)ccc(-n2c3ccccc3c3ccc4oc5ccccc5c4c32)c1C#N ; N#Cc1c(-c2nc(-c3ccccc3)nc(-c3ccccc3)n2)ccc(-n2c3ccccc3c3cc4c(cc32)oc2ccccc24)c1C#N]

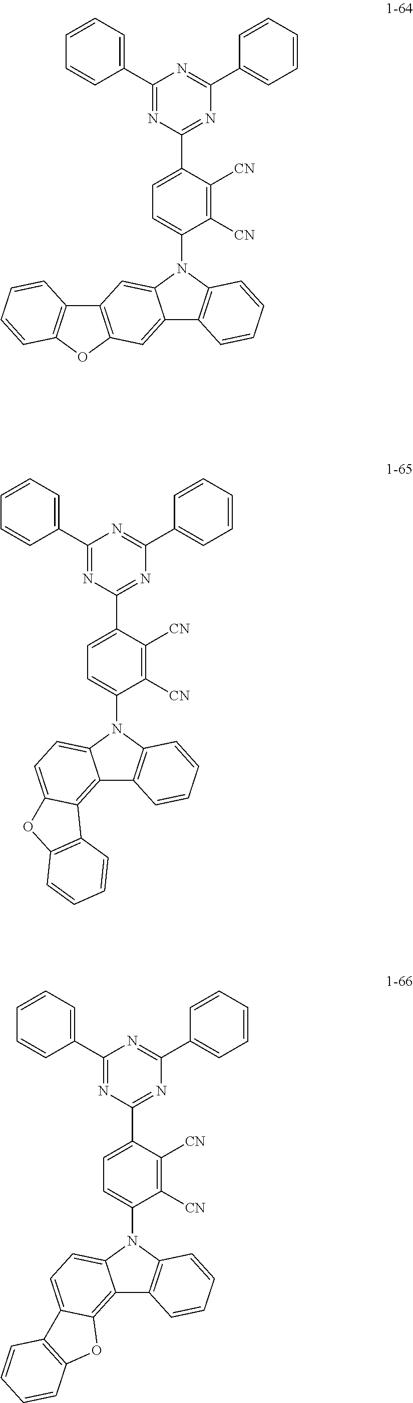 [SMILES: N#Cc1c(-c2nc(-c3ccccc3)nc(-c3ccccc3)n2)ccc(-n2c3ccccc3c3cc4oc5ccccc5c4cc32)c1C#N ; N#Cc1c(-c2nc(-c3ccccc3)nc(-c3ccccc3)n2)ccc(-n2c3ccccc3c3c4c(ccc32)oc2ccccc24)c1C#N ; N#Cc1c(-c2nc(-c3ccccc3)nc(-c3ccccc3)n2)ccc(-n2c3ccccc3c3c4oc5ccccc5c4ccc32)c1C#N]

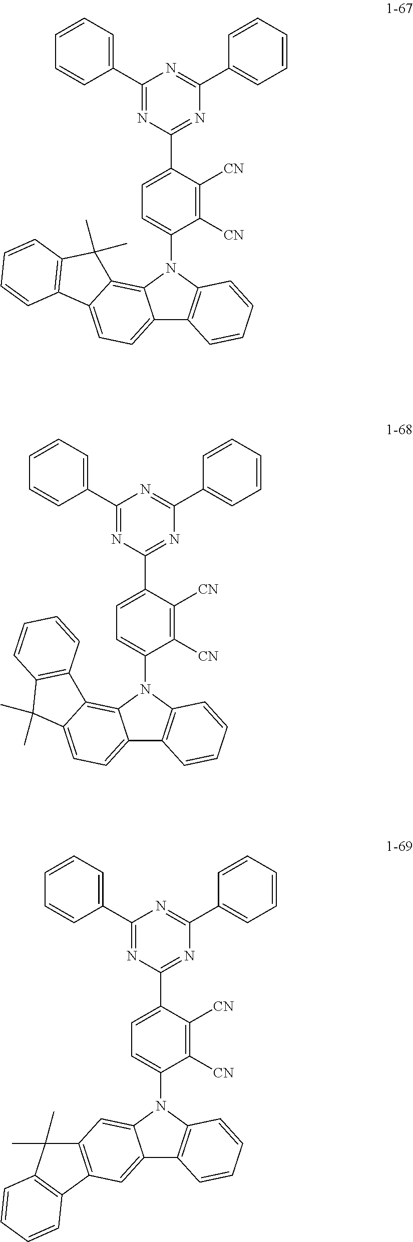 [SMILES: CC1(C)c2ccccc2-c2ccc3c4ccccc4n(-c4ccc(-c5nc(-c6ccccc6)nc(-c6ccccc6)n5)c(C#N)c4C#N)c3c21 ; CC1(C)c2ccccc2-c2c1ccc1c3ccccc3n(-c3ccc(-c4nc(-c5ccccc5)nc(-c5ccccc5)n4)c(C#N)c3C#N)c21 ; CC1(C)c2ccccc2-c2cc3c4ccccc4n(-c4ccc(-c5nc(-c6ccccc6)nc(-c6ccccc6)n5)c(C#N)c4C#N)c3cc21]

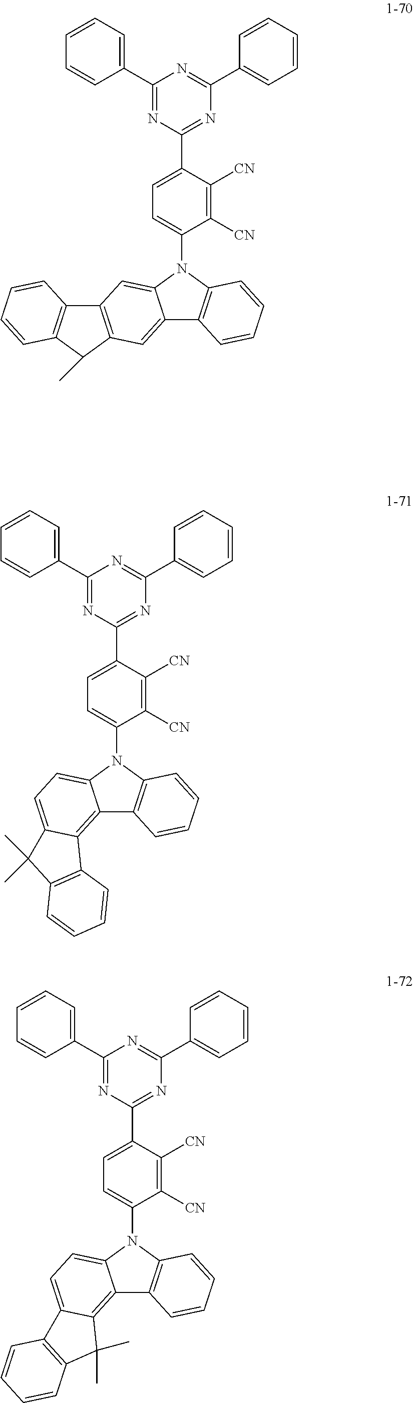 [SMILES: CC1c2ccccc2-c2cc3c(cc21)c1ccccc1n3-c1ccc(-c2nc(-c3ccccc3)nc(-c3ccccc3)n2)c(C#N)c1C#N ; CC1(C)c2ccccc2-c2c1ccc1c2c2ccccc2n1-c1ccc(-c2nc(-c3ccccc3)nc(-c3ccccc3)n2)c(C#N)c1C#N ; CC1(C)c2ccccc2-c2ccc3c(c21)c1ccccc1n3-c1ccc(-c2nc(-c3ccccc3)nc(-c3ccccc3)n2)c(C#N)c1C#N]

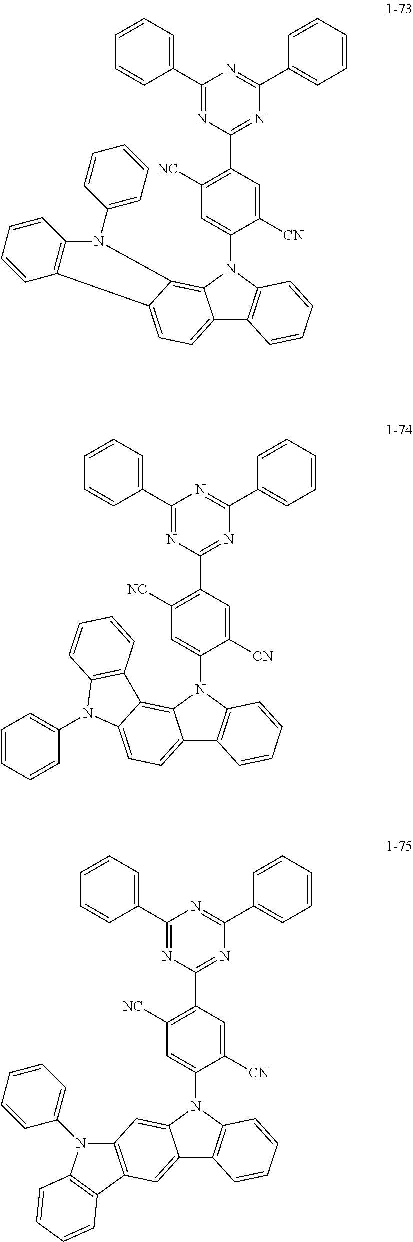 [SMILES: N#Cc1cc(-n2c3ccccc3c3ccc4c5ccccc5n(-c5ccccc5)c4c32)c(C#N)cc1-c1nc(-c2ccccc2)nc(-c2ccccc2)n1 ; N#Cc1cc(-n2c3ccccc3c3ccc4c(c5ccccc5n4-c4ccccc4)c32)c(C#N)cc1-c1nc(-c2ccccc2)nc(-c2ccccc2)n1 ; N#Cc1cc(-n2c3ccccc3c3cc4c5ccccc5n(-c5ccccc5)c4cc32)c(C#N)cc1-c1nc(-c2ccccc2)nc(-c2ccccc2)n1]

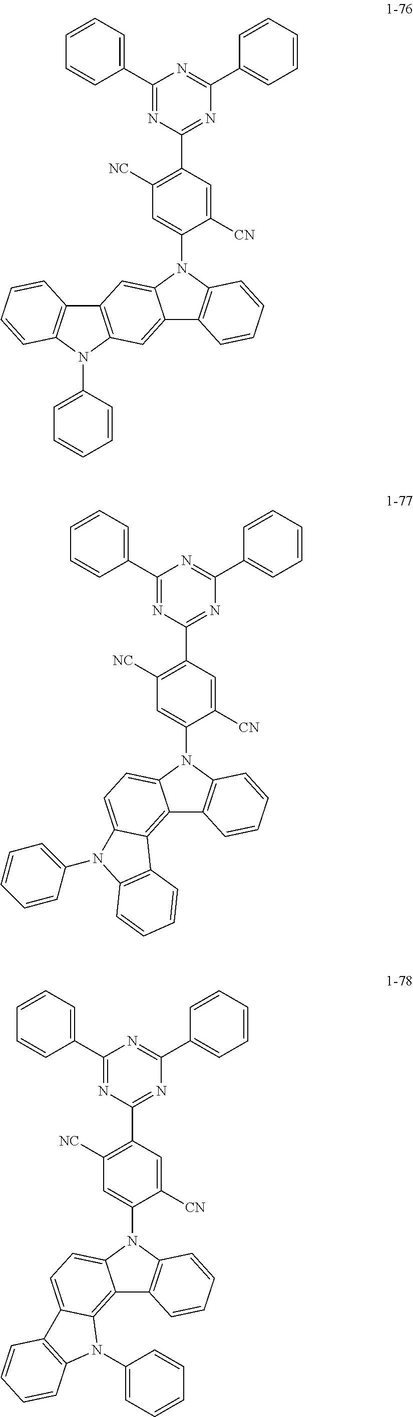 [SMILES: N#Cc1cc(-n2c3ccccc3c3cc4c(cc32)c2ccccc2n4-c2ccccc2)c(C#N)cc1-c1nc(-c2ccccc2)nc(-c2ccccc2)n1 ; N#Cc1cc(-n2c3ccccc3c3c4c5ccccc5n(-c5ccccc5)c4ccc32)c(C#N)cc1-c1nc(-c2ccccc2)nc(-c2ccccc2)n1 ; N#Cc1cc(-n2c3ccccc3c3c2ccc2c4ccccc4n(-c4ccccc4)c23)c(C#N)cc1-c1nc(-c2ccccc2)nc(-c2ccccc2)n1]

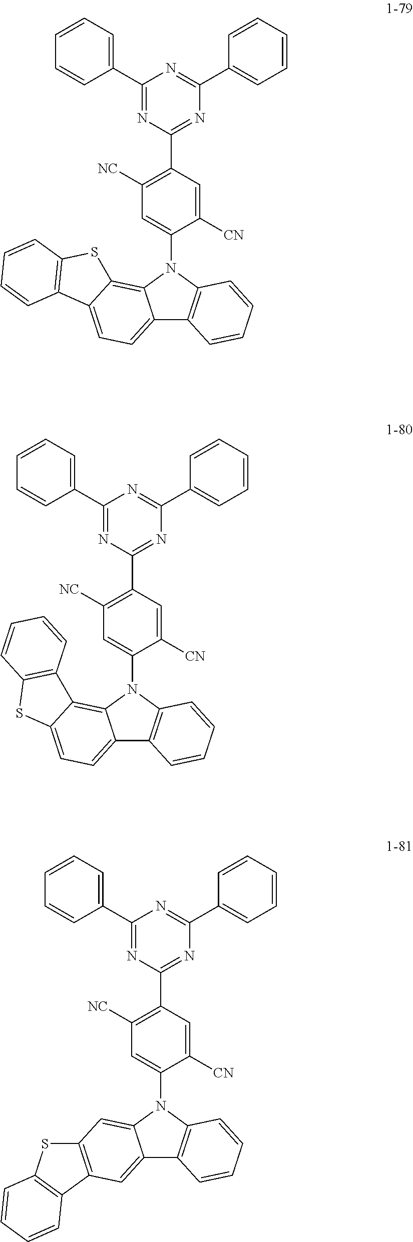 [SMILES: N#Cc1cc(-n2c3ccccc3c3ccc4c5ccccc5sc4c32)c(C#N)cc1-c1nc(-c2ccccc2)nc(-c2ccccc2)n1 ; N#Cc1cc(-n2c3ccccc3c3ccc4sc5ccccc5c4c32)c(C#N)cc1-c1nc(-c2ccccc2)nc(-c2ccccc2)n1 ; N#Cc1cc(-n2c3ccccc3c3cc4c(cc32)sc2ccccc24)c(C#N)cc1-c1nc(-c2ccccc2)nc(-c2ccccc2)n1]

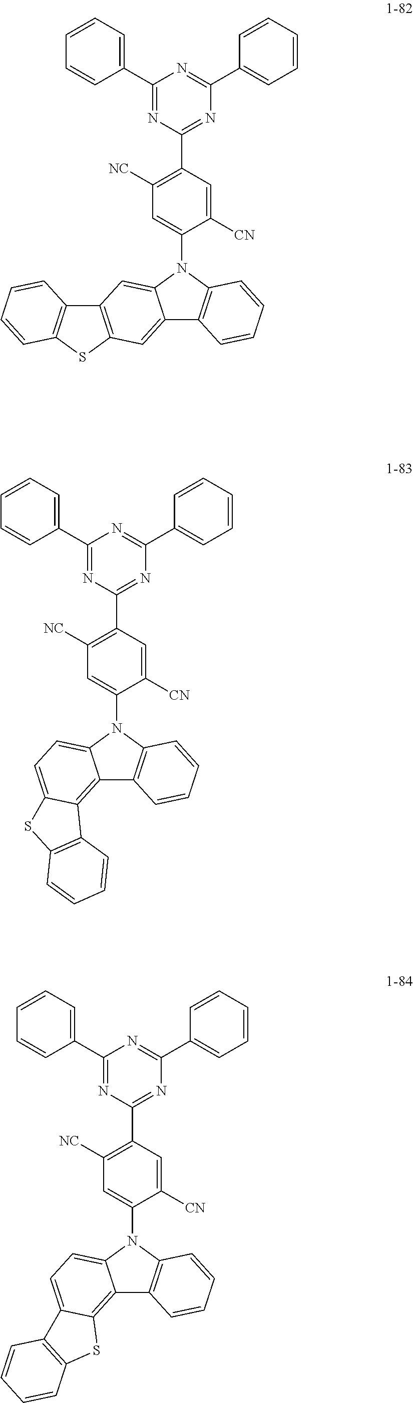 [SMILES: N#Cc1cc(-n2c3ccccc3c3cc4sc5ccccc5c4cc32)c(C#N)cc1-c1nc(-c2ccccc2)nc(-c2ccccc2)n1 ; N#Cc1cc(-n2c3ccccc3c3c4c(ccc32)sc2ccccc24)c(C#N)cc1-c1nc(-c2ccccc2)nc(-c2ccccc2)n1 ; N#Cc1cc(-n2c3ccccc3c3c4sc5ccccc5c4ccc32)c(C#N)cc1-c1nc(-c2ccccc2)nc(-c2ccccc2)n1]

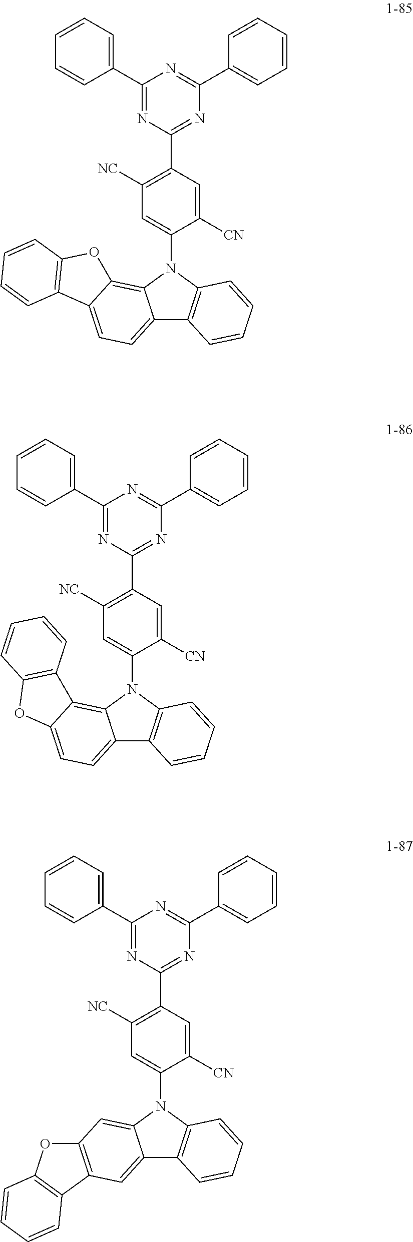 [SMILES: N#Cc1cc(-n2c3ccccc3c3ccc4c5ccccc5oc4c32)c(C#N)cc1-c1nc(-c2ccccc2)nc(-c2ccccc2)n1 ; N#Cc1cc(-n2c3ccccc3c3ccc4oc5ccccc5c4c32)c(C#N)cc1-c1nc(-c2ccccc2)nc(-c2ccccc2)n1 ; N#Cc1cc(-n2c3ccccc3c3cc4c(cc32)oc2ccccc24)c(C#N)cc1-c1nc(-c2ccccc2)nc(-c2ccccc2)n1]

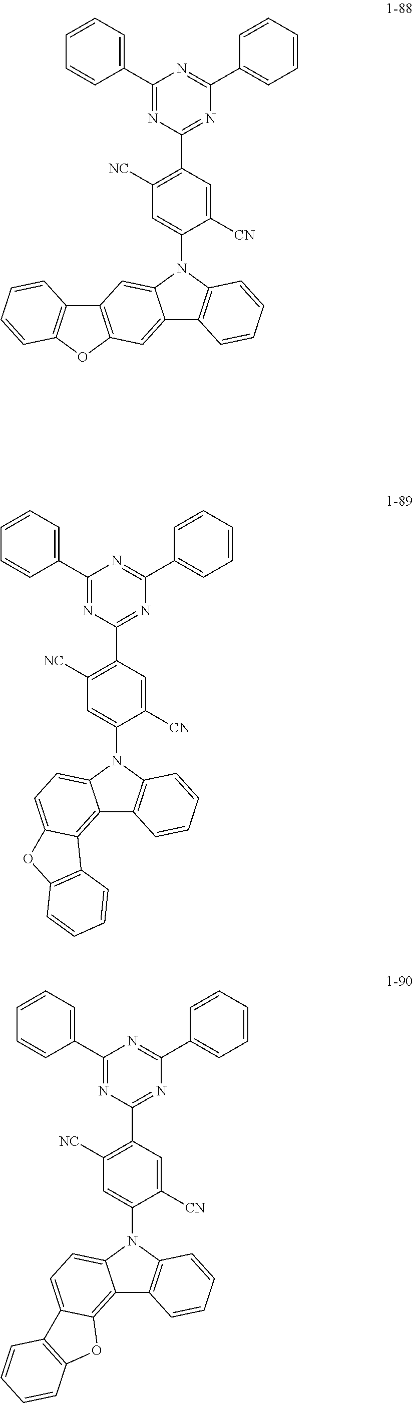 [SMILES: N#Cc1cc(-n2c3ccccc3c3cc4oc5ccccc5c4cc32)c(C#N)cc1-c1nc(-c2ccccc2)nc(-c2ccccc2)n1 ; N#Cc1cc(-n2c3ccccc3c3c4c(ccc32)oc2ccccc24)c(C#N)cc1-c1nc(-c2ccccc2)nc(-c2ccccc2)n1 ; N#Cc1cc(-n2c3ccccc3c3c4oc5ccccc5c4ccc32)c(C#N)cc1-c1nc(-c2ccccc2)nc(-c2ccccc2)n1]

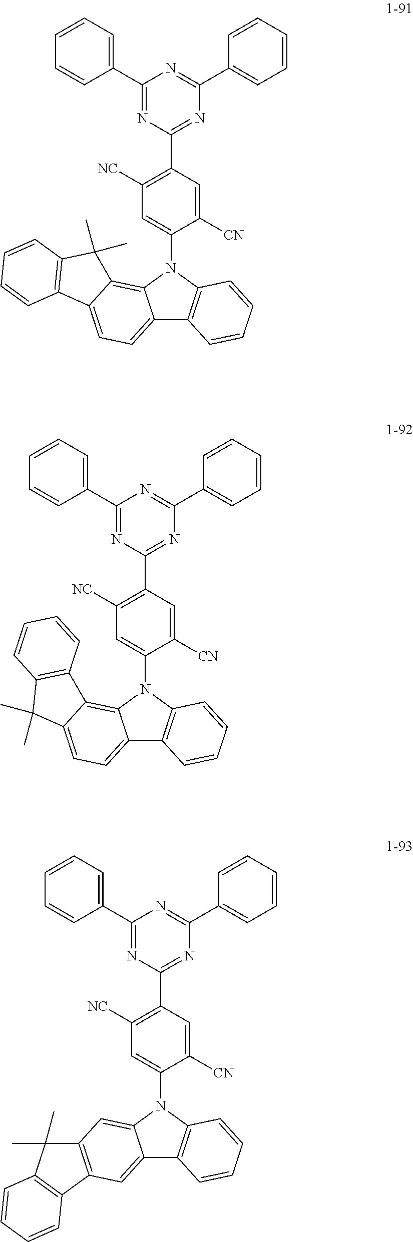 [SMILES: CC1(C)c2ccccc2-c2ccc3c4ccccc4n(-c4cc(C#N)c(-c5nc(-c6ccccc6)nc(-c6ccccc6)n5)cc4C#N)c3c21 ; CC1(C)c2ccccc2-c2c1ccc1c3ccccc3n(-c3cc(C#N)c(-c4nc(-c5ccccc5)nc(-c5ccccc5)n4)cc3C#N)c21 ; CC1(C)c2ccccc2-c2cc3c4ccccc4n(-c4cc(C#N)c(-c5nc(-c6ccccc6)nc(-c6ccccc6)n5)cc4C#N)c3cc21]

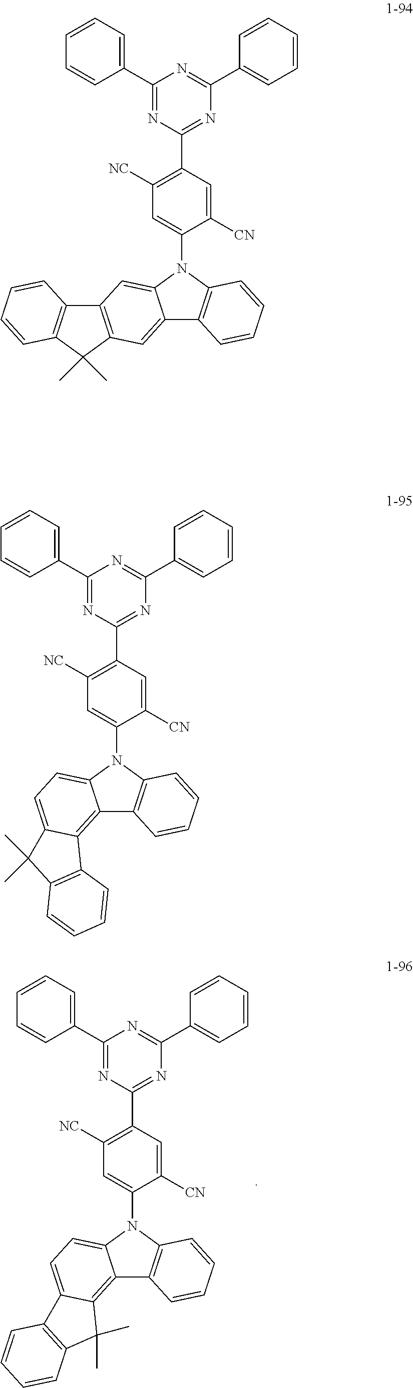 [SMILES: CC1(C)c2ccccc2-c2cc3c(cc21)c1ccccc1n3-c1cc(C#N)c(-c2nc(-c3ccccc3)nc(-c3ccccc3)n2)cc1C#N ; CC1(C)c2ccccc2-c2c1ccc1c2c2ccccc2n1-c1cc(C#N)c(-c2nc(-c3ccccc3)nc(-c3ccccc3)n2)cc1C#N ; CC1(C)c2ccccc2-c2ccc3c(c21)c1ccccc1n3-c1cc(C#N)c(-c2nc(-c3ccccc3)nc(-c3ccccc3)n2)cc1C#N]

As described above, since the delayed fluorescent material has an energy level bandgap (e.g. equal to or less than about 0.3 eV) between the singlet energy level and the triplet energy level so that triplet exciton energy is converted upwardly to its singlet exciton energy by RISC, it can implement high quantum efficiency. However, the delayed fluorescent material has very wide FWHM (full width at half maximum) so that it has disadvantage in terms of color purity.

In order to solve the disadvantage in term of color purity of the delayed florescent material, the EML 240 includes the second compound FD of fluorescent material to implement hyper-fluorescence. The second compound FD of the fluorescent material in the EML 240 may have the following structure of Formula 4:

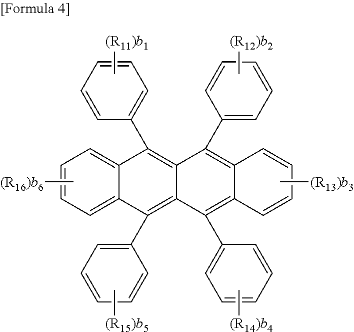

In Formula 4, each of R₁₁ to R₁₆ is independently selected from the group consisting of deuterium, tritium, a C₁-C₂₀ alkyl group, a C₆-C₃₀ aryl group and a C₃-C₄₀ hetero aryl group, or adjacent two of R₁₁ to R₁₆ form a fused ring; each of b₁, b₂, b₄ and b₅ is independently an integer of 0 to 5; and each of b₃ and b₆ is independently an integer of 0 to 4.

The fused ring may be a C₆-C₂₀ fused alicyclic ring, a C₄-C₂₀ fused hetero alicyclic ring, a C₆-C₂₀ fused aromatic ring or a C₄-C₂₀ fused hetero aromatic ring. Each of the alkyl group, the aryl group and the hetero aryl group may be independently substituted or unsubstituted. As an example, each of b₁ to b₆ may be independently an integer of 0 to 3, each of RH to R₁₆ may be independently, but is not limited to, methyl, isopropyl or tertiary butyl.

More particularly, the second compound FD of the fluorescent material may be selected from the following compounds having the structure of Formula 5:

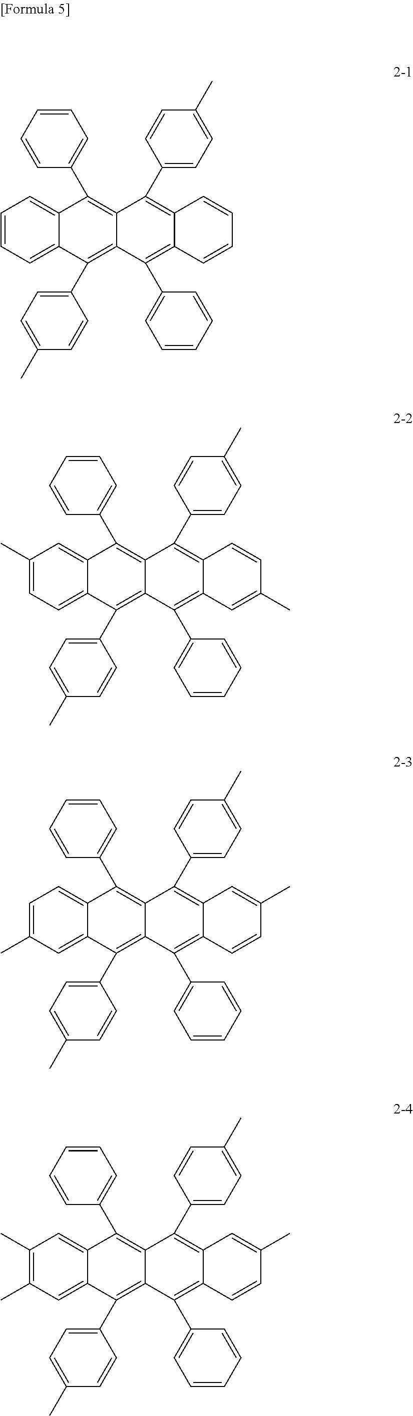

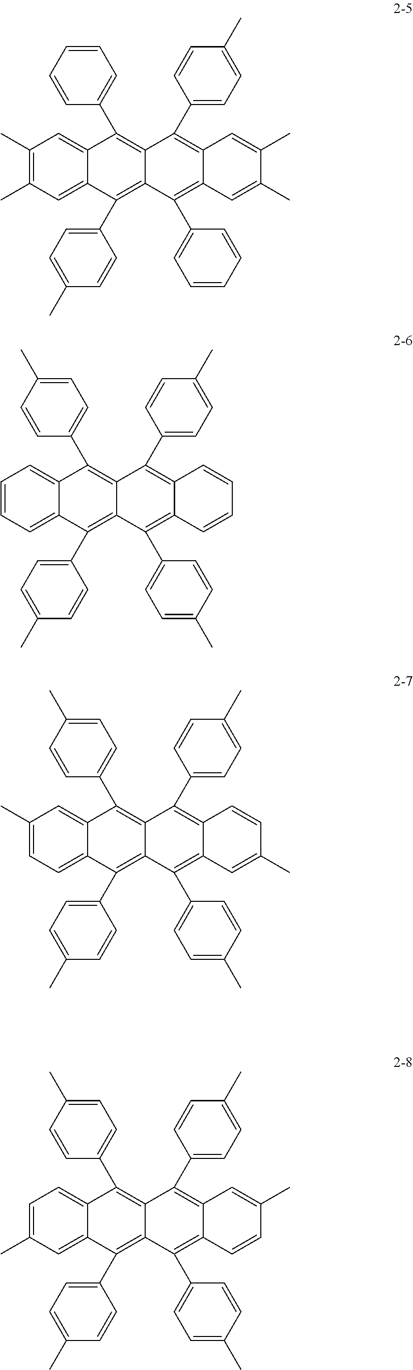

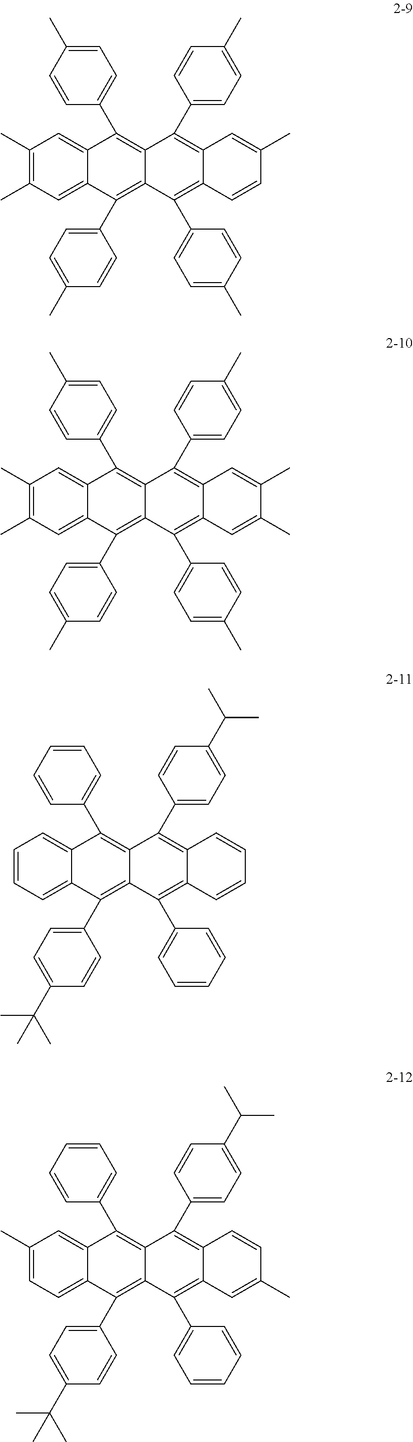

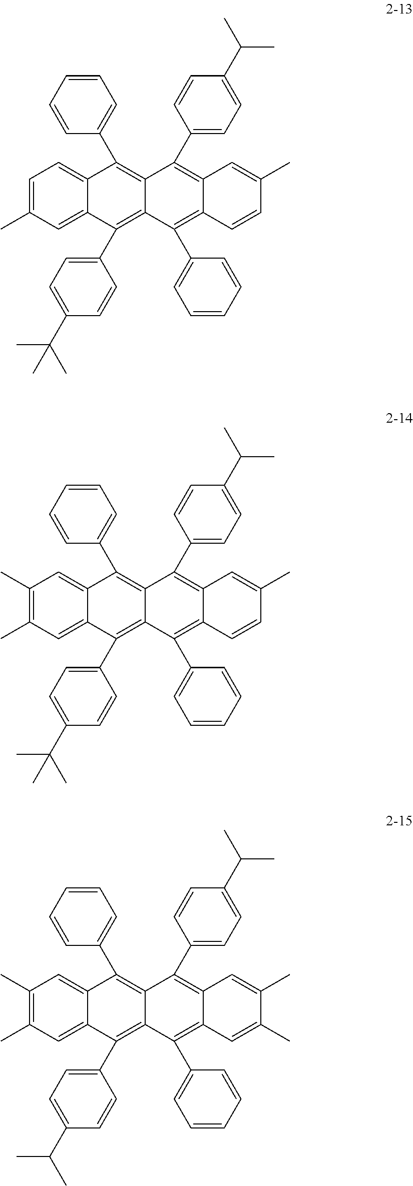

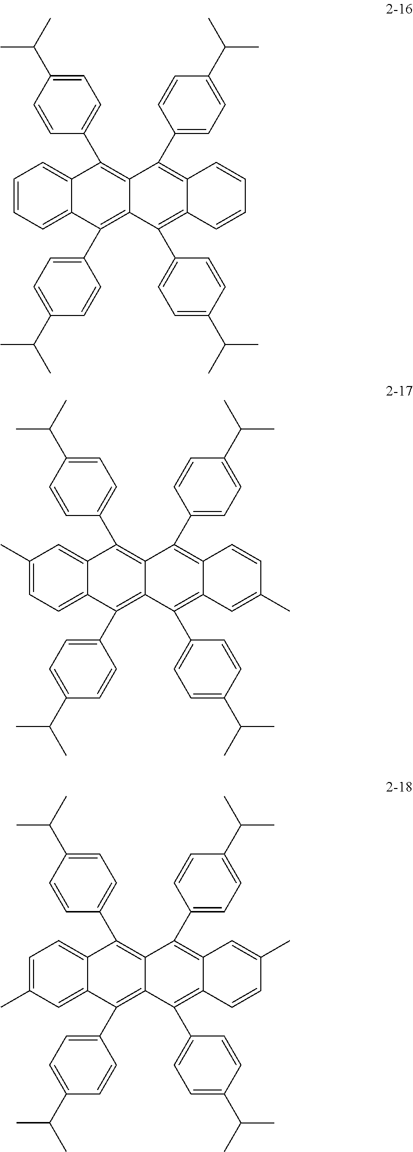

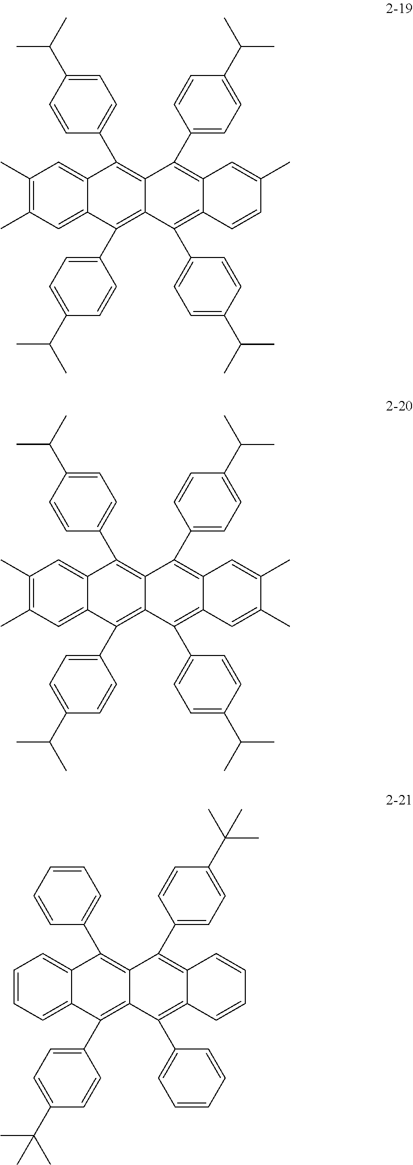

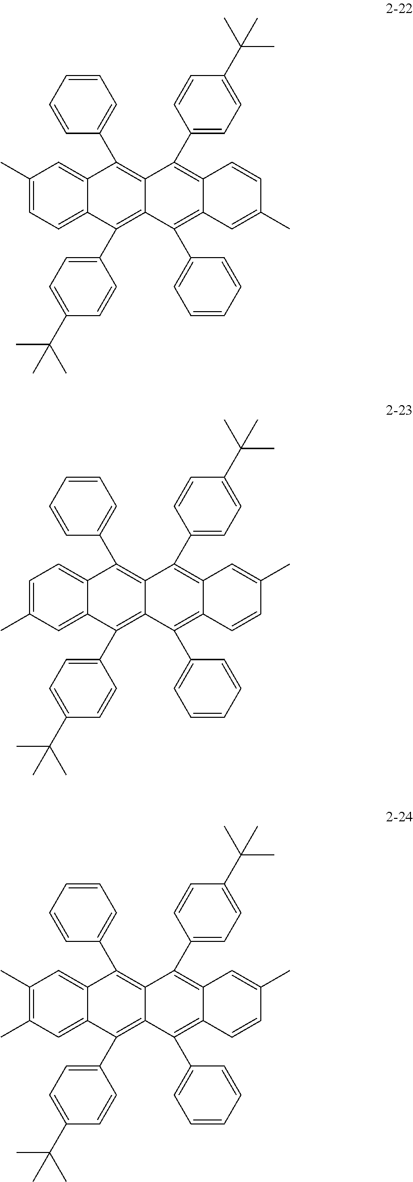

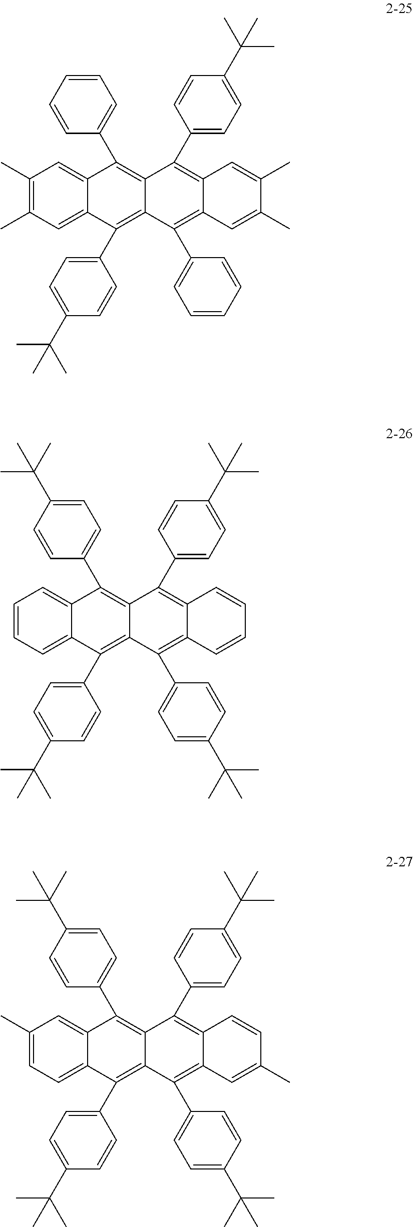

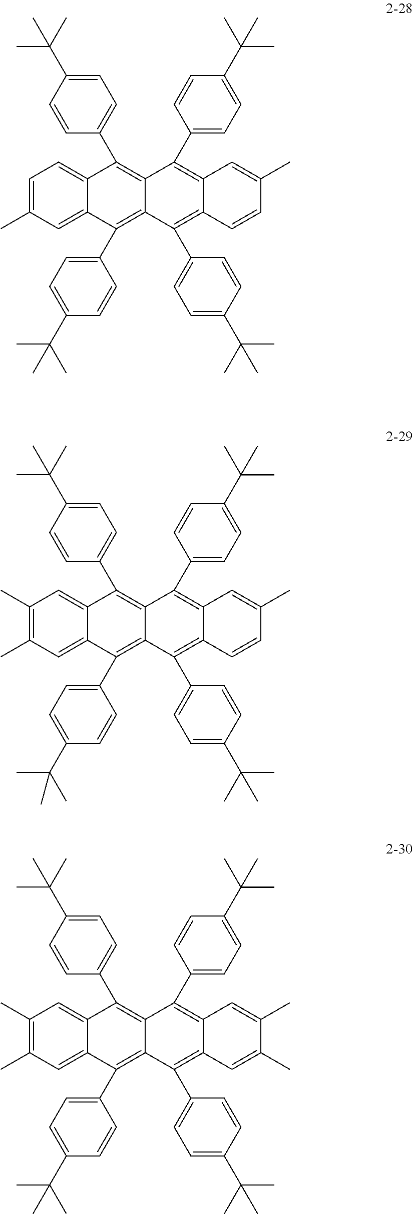

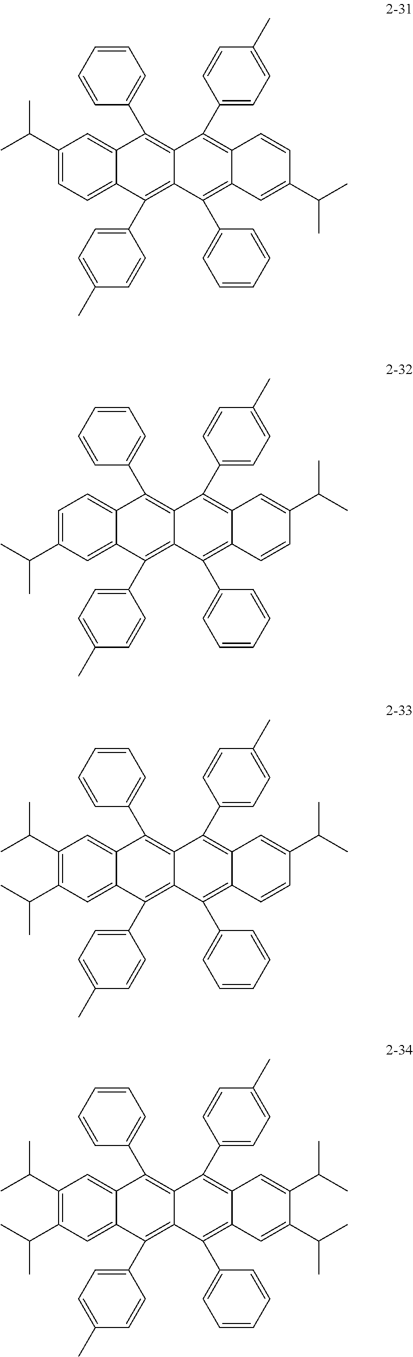

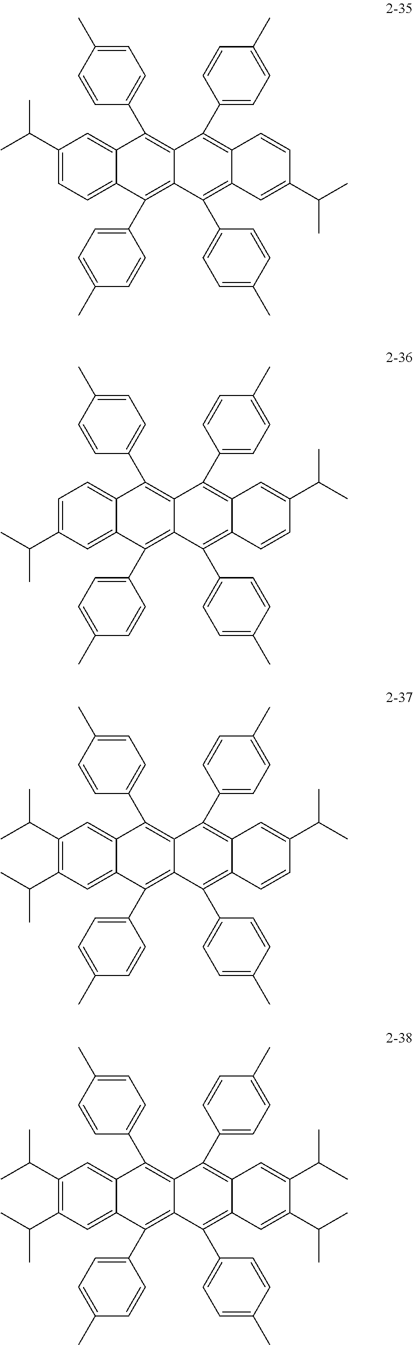

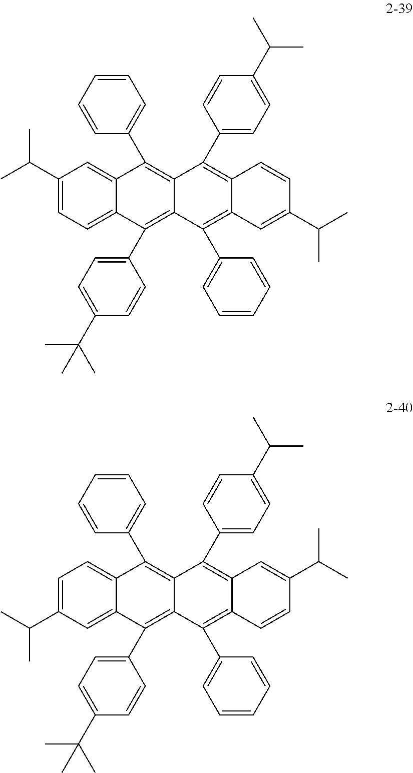

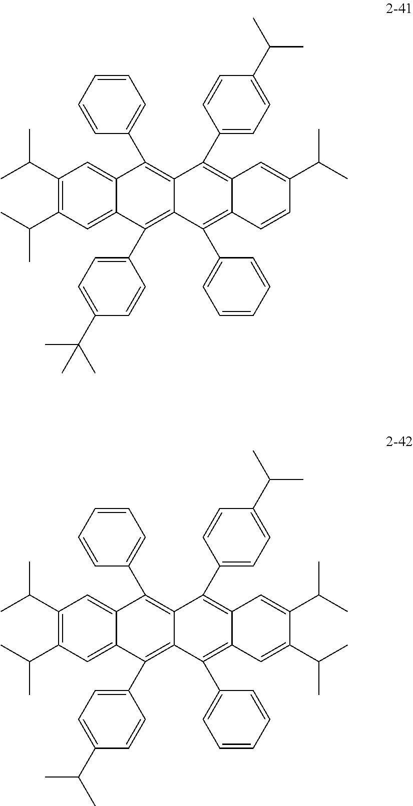

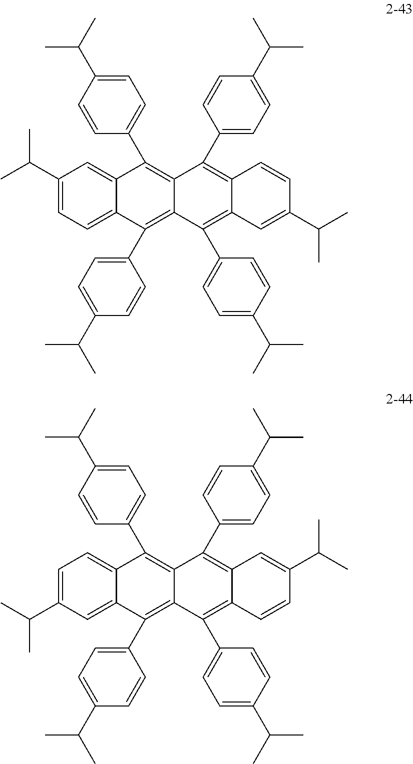

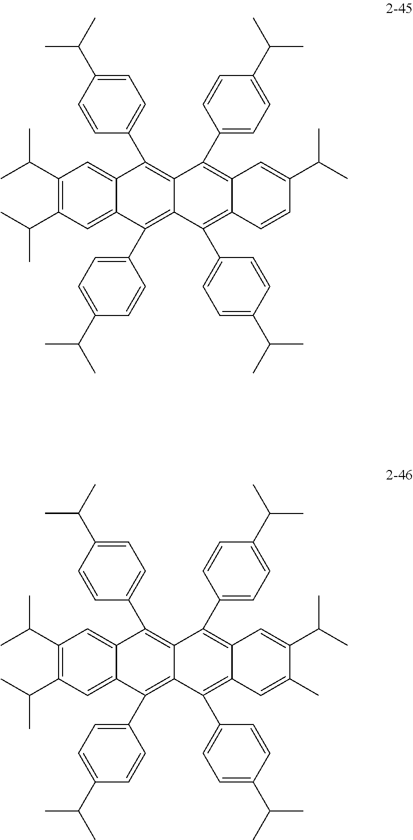

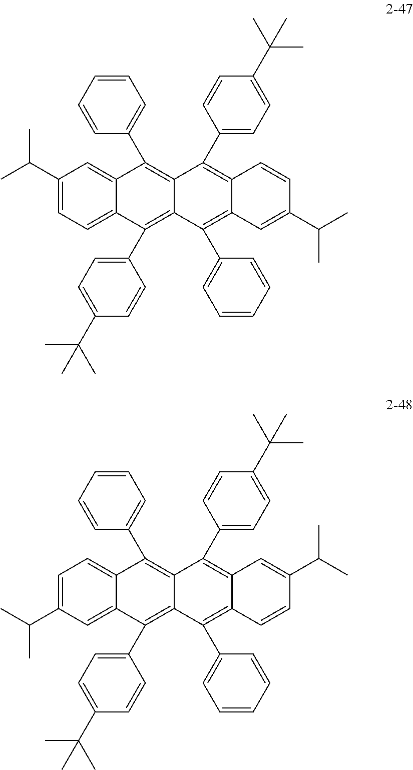

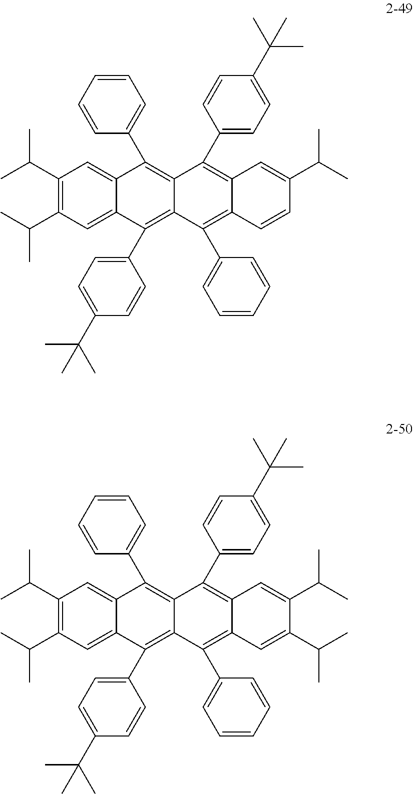

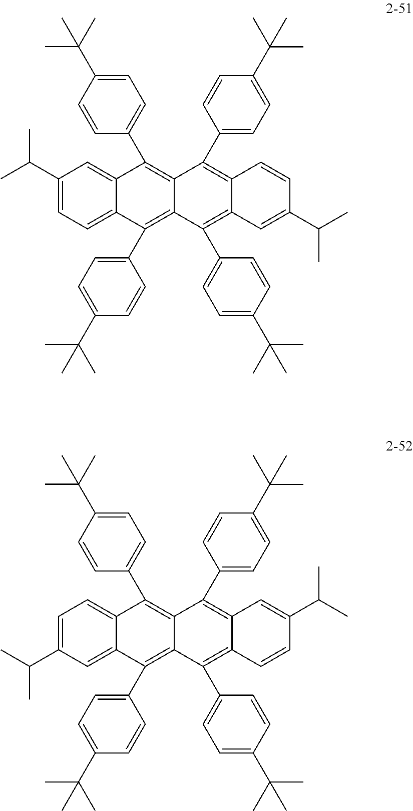

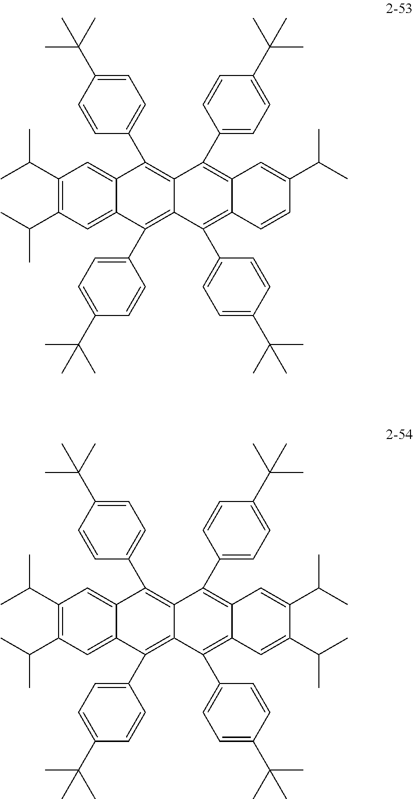

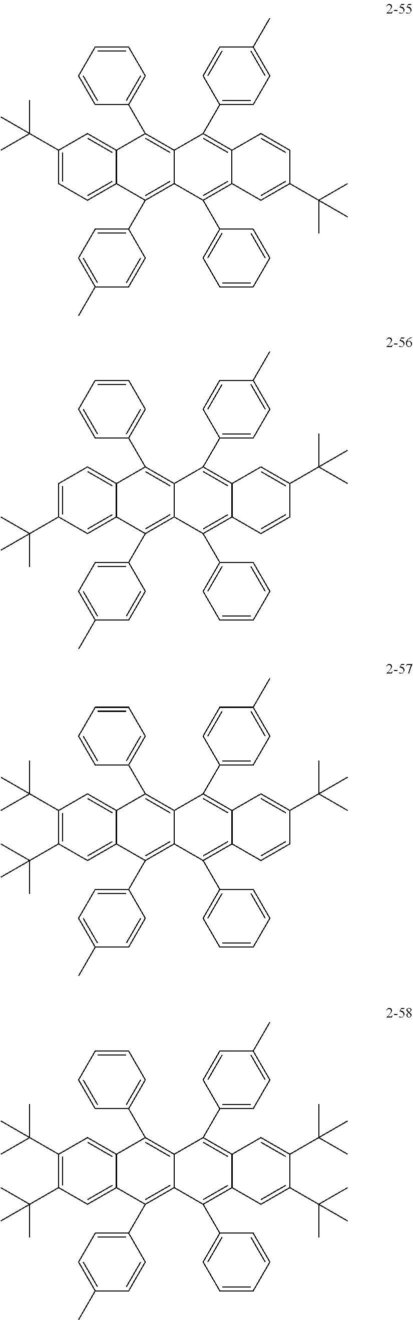

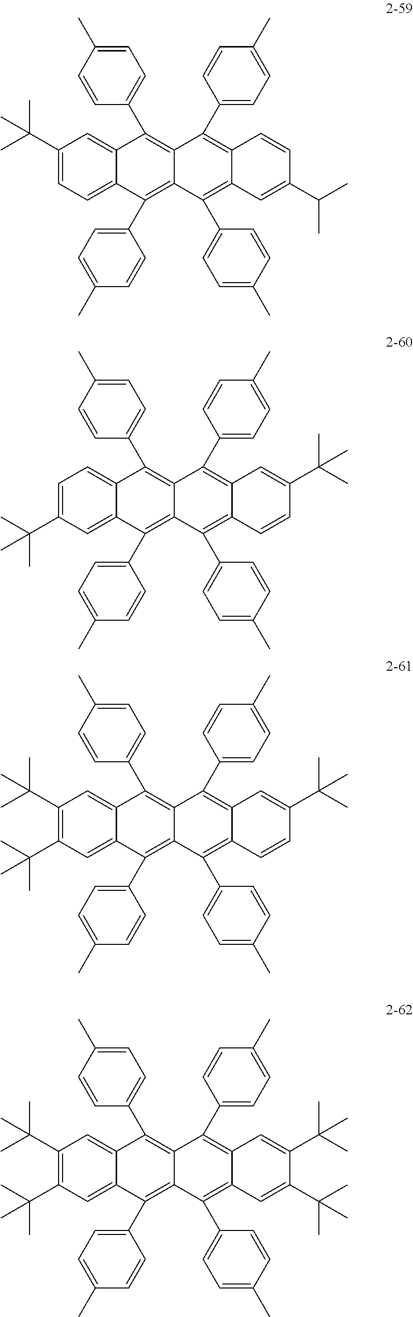

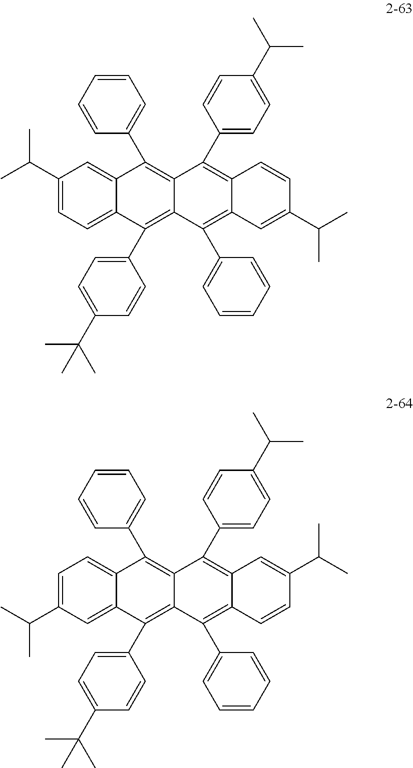

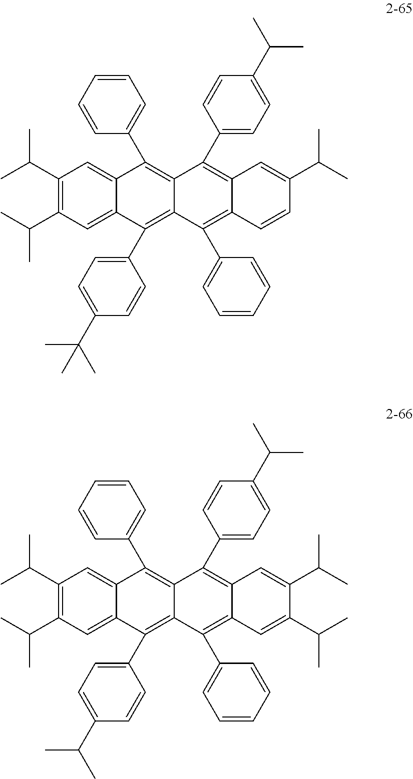

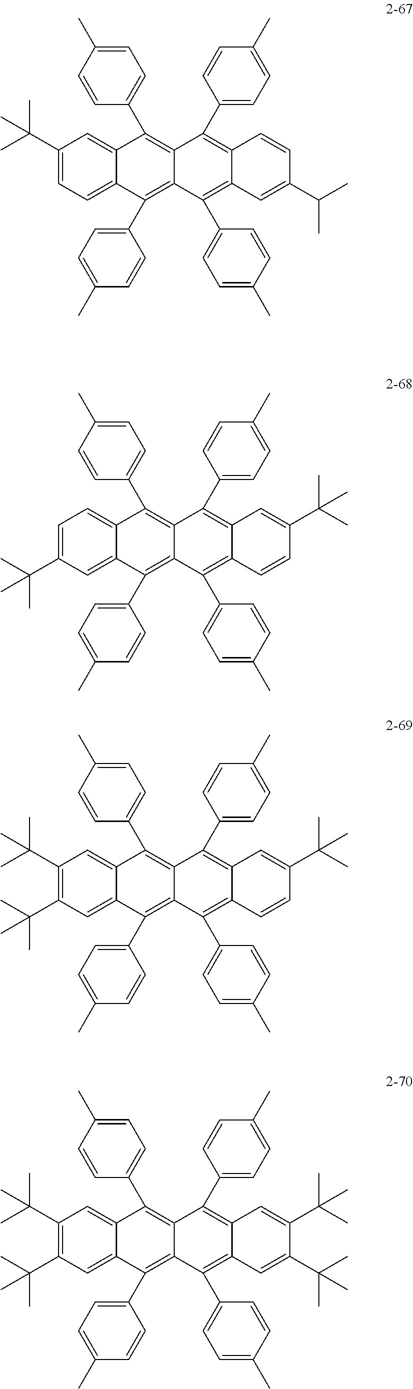

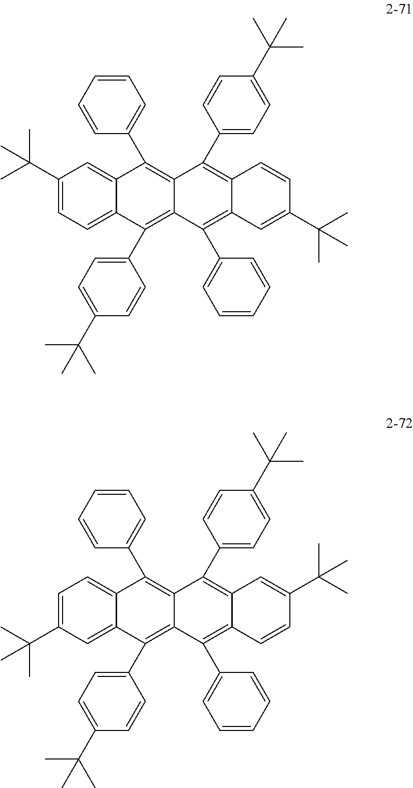

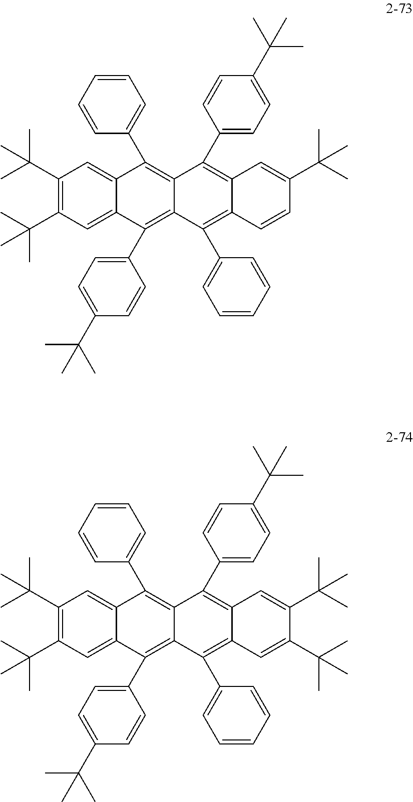

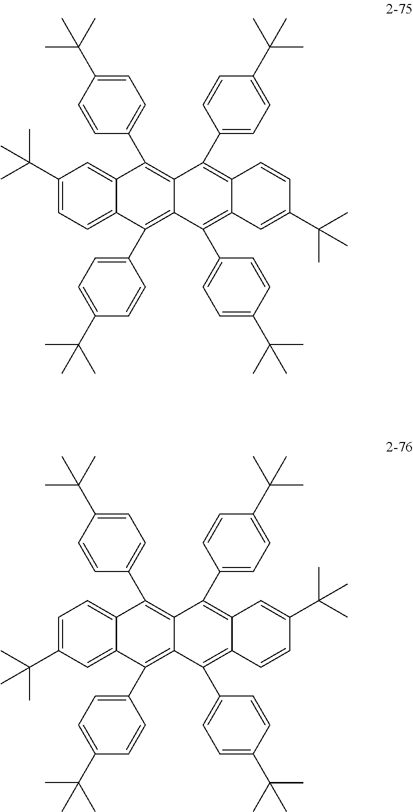

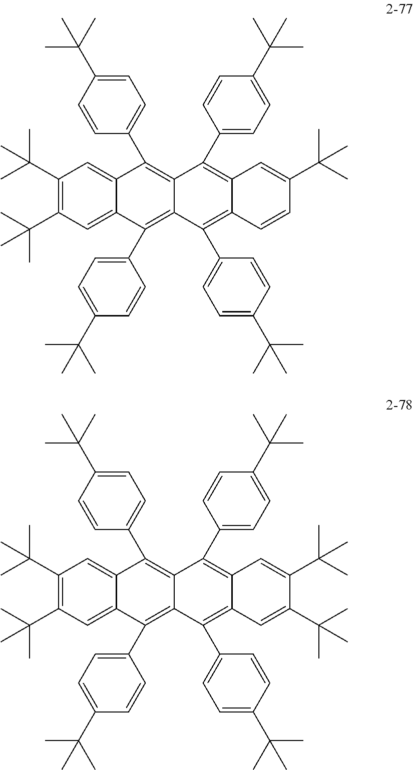

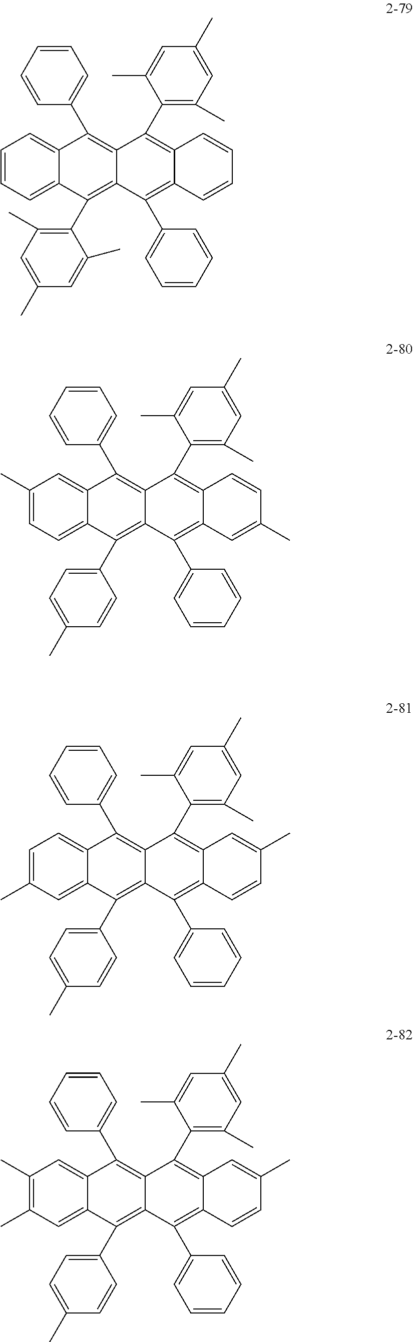

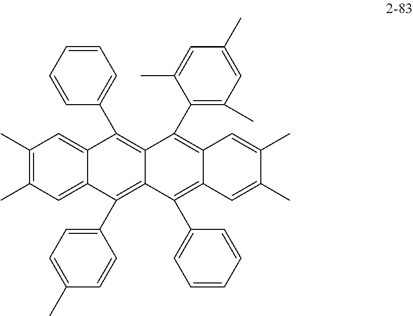

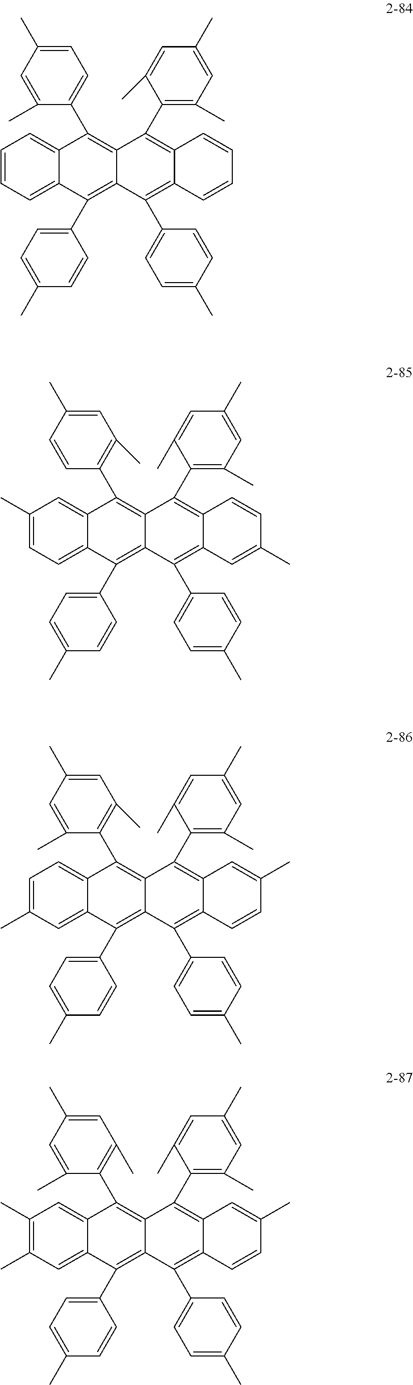

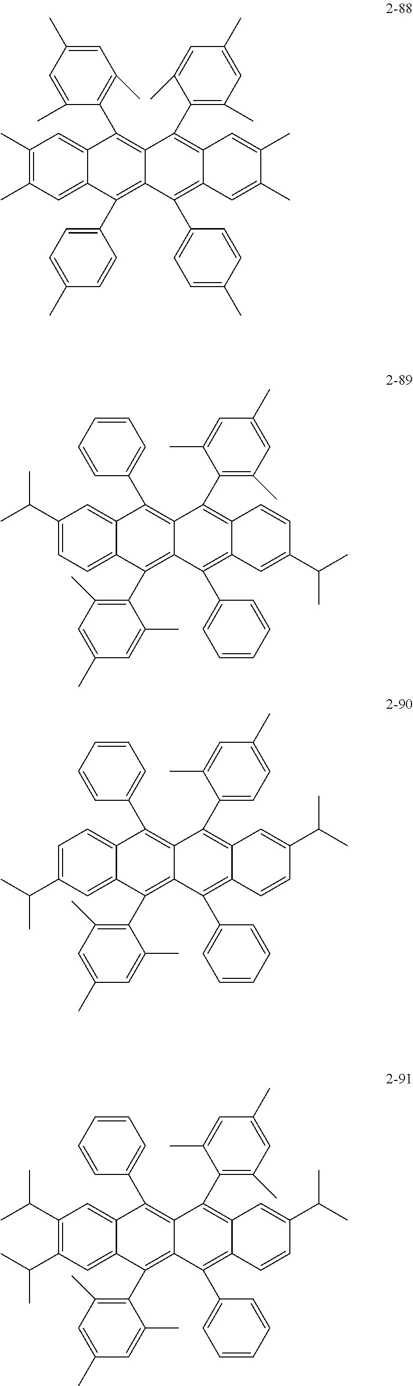

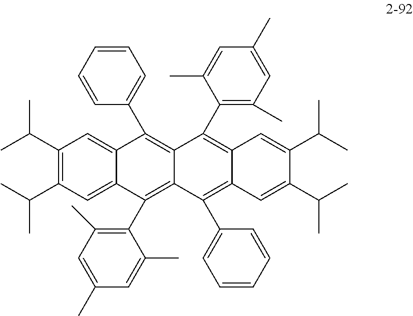 [SMILES: Cc1cc(C)c(-c2c3cc(C(C)C)c(C(C)C)cc3c(-c3ccccc3)c3c(-c4c(C)cc(C)cc4C)c4cc(C(C)C)c(C(C)C)cc4c(-c4ccccc4)c23)c(C)c1]

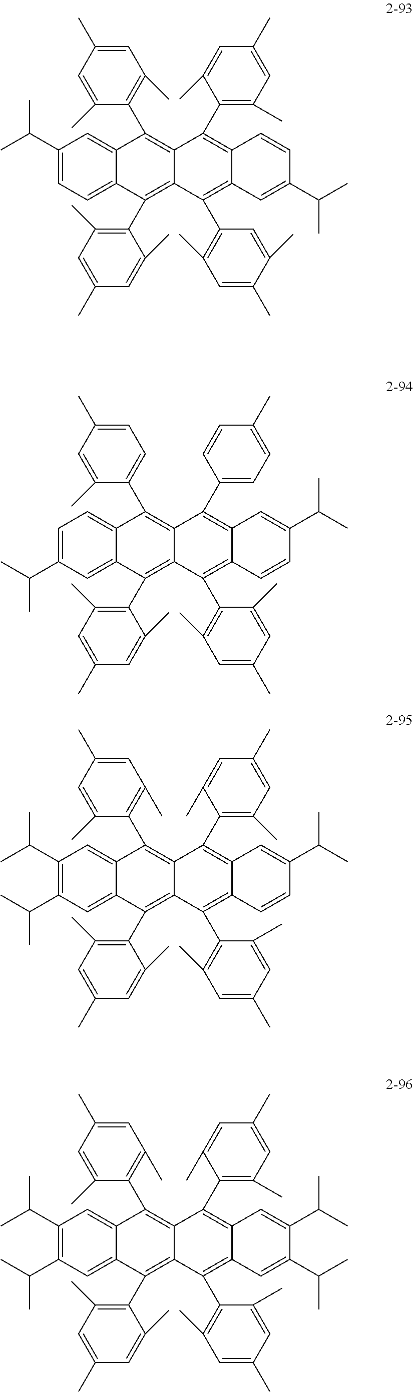

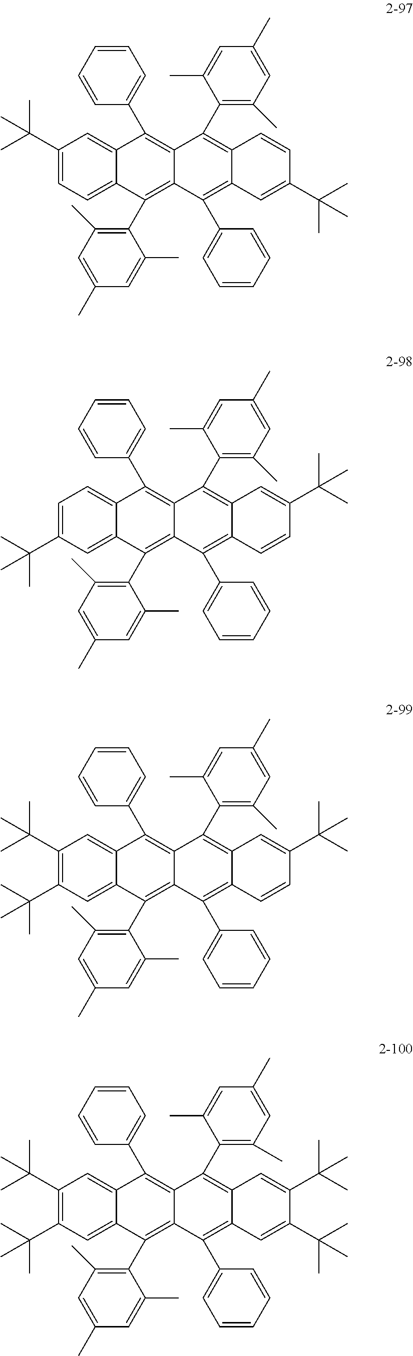

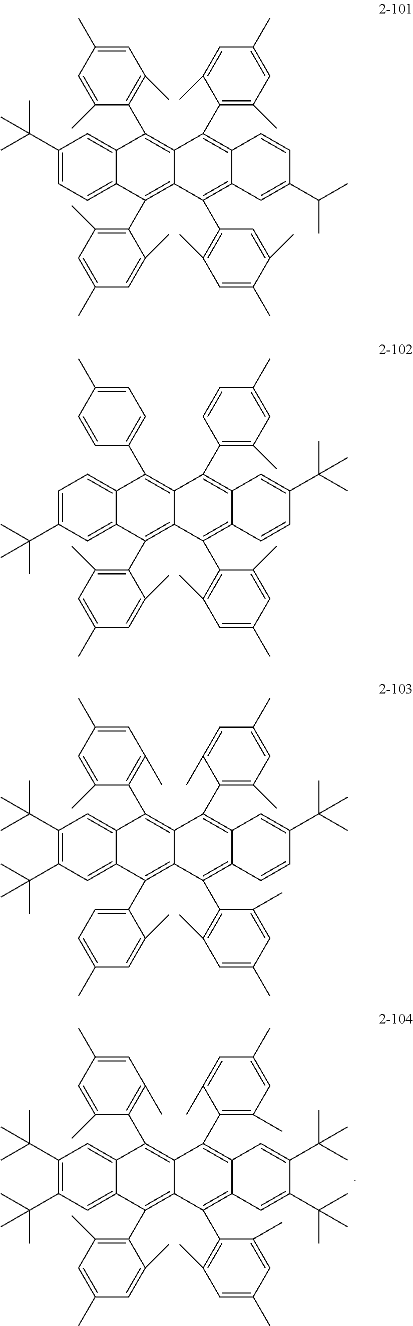

In addition, the third compound H in the EML 240 may comprise, but is not limited to, 9-(3-(9H-carbazol-9-yl)phenyl)-9H-carbazole-3-carbonitrile (mCP-CN), 4,4′-bis(N-carbazolyl)-1,1′-biphenyl (CBP), 3,3′-bis(N-carbazolyl)-1,1′-biphenyl (mCBP), 1,3-Bis(carbazol-9-yl)benzene (mCP), Bis[2-(diphenylphosphino)phenyl]ether oxide (DPEPO), 2,8-bis(diphenylphosphoryl)dibenzothiophene (PPT), 1,3,5-Tri[(3-pyridyl)-phen-3-yl]benzene (TmPyPB), 2,6-Di(9H-carbazol-9-yl)pyridine (PYD-2Cz), 2,8-di(9H-carbazol-9-yl)dibenzothiophene (DCzDBT), 3′,5′-Di(carbazol-9-yl)-[1,1′-biphenyl]-3,5-dicarbonitrile (DCzTPA), 4′-(9H-carbazol-9-yl)biphenyl-3,5-dicarbonitrile(4′-(9H-carbazol-9-yl)biphenyl-3,5-dicarbonitrile (pCzB-2CN), 3′-(9H-carbazol-9-yl)biphenyl-3,5-dicarbonitrile (mCzB-2CN), Diphenyl-4-triphenylsilylphenyl-phosphine oxide (TSPO1), 9-(9-phenyl-9H-carbazol-6-yl)-9H-carbazole (CCP), 4-(3-(triphenylene-2-yl)phenyl)dibenzo[b,d]thiophene, 9-(4-(9H-carbazol-9-yl)phenyl)-9H-3,9′-bicarbazole, 9-(3-(9H-carbazol-9-yl)phenyl)-9H-3,9′-bicarbazole and/or 9-(6-(9H-carbazol-9-yl)pyridin-3-yl)-9H-3,9′-bicarbazole).

Since the EML 240 in the OLED D1 includes the first compound DF and the second compound FD and exciton energy of the first compound DF can be transferred to the second compound FD, the OLED D1 can implement narrow FWHM and high luminous efficiency.

In addition, when an energy level of the first compound DF and an energy level of the second compound FD may satisfy predetermined conditions, exciton energy efficiency from the first compound DF to the second compound FD can be significantly increased.

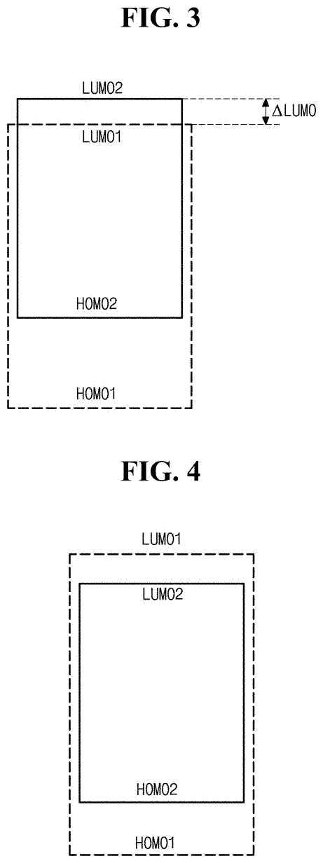

With referring to FIG. 3, which is a schematic diagram illustrating the energy level relationship between the first compound and the second compound in the OLED D1, an lowest unoccupied molecular orbital (LUMO) energy level LUMO1 of the first compound is equal to or deeper (lower) than a LUMO energy level LUMO2 of the second compound. For example, the energy level bandgap between the LUMO energy level LUMO1 of the first compound and the LUMO energy level LUMO2 of the second compound is equal to or less than 0.2 eV. In other words, the LUMO energy level LUMO1 of the first compound and the LUMO energy level LUMO2 of the second compound satisfy the following relationship:

ΔLUMO(=LUMO2−LUMO1)1≤0.2 eV.

In addition, a highest occupied molecular orbital (HOMO) energy level HOMO1 of the first compound may be equal to or deeper than a HOMO energy level HOMO2 of the second compound. Also, the first compound may have energy level bandgap between the HOMO energy level and the LUMO energy level higher than energy level bandgap of the second compound. For example, the first compound may have energy level bandgap between about 2.0 eV and about 2.8 eV, for example, about 2.4 eV and about 2.6 eV.

As described above, the first compound DF of the delayed fluorescent material can utilize both the singlet exciton energy and the triplet exciton energy in the luminous process. Therefore, when the EML 240 includes the first compound DF and the second compound FD, the exciton energy of the first compound DF can be transferred to the second compound FD in which ultimate emission is occurred, and therefore, the OLED D1 can improve its luminous lifespan and color purity.

On the other hand, even when the EML includes the delayed fluorescent material and the fluorescent material, the OLED cannot improve its luminous efficiency and color purity sufficiency if the energy level relationship above is not satisfied. For example, as indicated in FIG. 4, when the LUMO energy level LUMO1 of the delayed fluorescent material is shallower (higher) than the LUMO energy level LUMO2 of the fluorescent material, the exciton energy generated in the host is not transferred to the delayed fluorescent material, but to the fluorescent material. Accordingly, the triplet exciton energy of the delayed fluorescent material is not utilized in the luminous process so that the luminous efficiency is decreased, and the luminous lifespan of the OLED is significantly reduced due to the emission in the delayed fluorescent material.

In the EML 240, the singlet energy level S₁^{DF} of the first compound is lower than the singlet energy level S₁^{H} of the third compound and is higher than the singlet energy level S₁^{FD} of the second compound. Also, the triplet energy level T₁^{DF} of the first compound is equal to or less than the triplet energy level T₁^{H} of the third compound and is higher than the triplet energy level T₁^{FD} of the second compound.

In addition, a HOMO energy level of the third compound is deeper (lower) than each of the HOMO energy levels of the first and second compounds, respectively, and a LUMO energy level of the third compound is shallower than each of the LUMO energy levels of the first and second compounds, respectively. For example, energy level bandgap between the HOMO energy level of the third compound and the HOMO energy level of the first compound, or energy level bandgap between the LUMO energy level of the third compound and the LUMO energy level of the first compound may be equal to or less than about 0.5 eV, for example, between about 0.1 eV to about 0.5 eV. In this case, the charges can be transported efficiently from the third compound to the first compound, and thereby enhancing the ultimate luminous efficiency in the EML 240.

In the EML 240, the contents of the first compound may be larger than the contents of the second compound and may be less than the contents of the third compound. In this case, exciton energy is efficiently transferred from the first compound to the second compound. As an example, the EML 240 may comprise, but is not limited to, the first compound between about 20 wt % and about 40 wt %, the second compound between about 0.1 wt % and about 5 wt %, and the third compound between about 60 wt % and about 75 wt %.

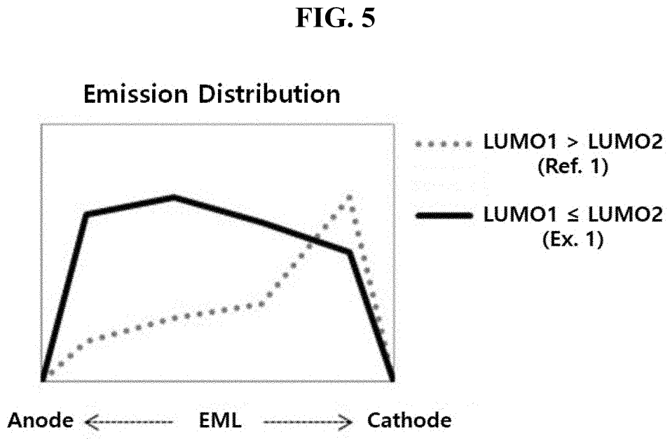

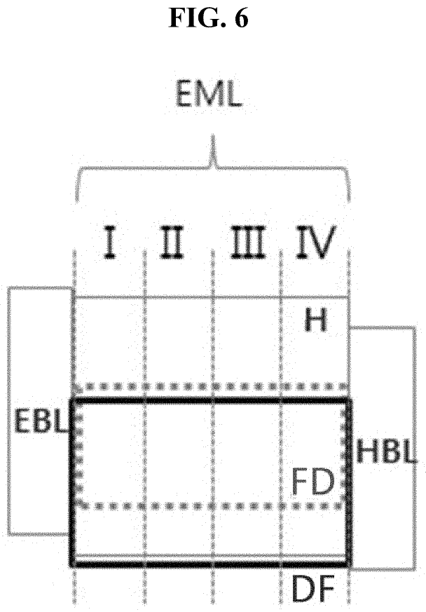

FIG. 5 is a schematic diagram illustrating emission distribution with regard to an energy level relationship between the first compound and the second compound in accordance with the present disclosure, and FIG. 6 is a schematic diagram illustrating a method for measuring the emission distribution in FIG. 5. When the LUMO energy level LUMO1 of the first compound is higher than the LUMO energy level of the second compound, as illustrated in FIG. 4, the emission distribution in the EML is biased to one side. On the other hand, when the LUMO energy level LUMO1 of the first compound is equal to or less than the LUMO energy level LUMO2 of the second compound, as illustrated in FIG. 3, the emission in the EML is evenly distributed.

When the LUMO energy level LUMO1 of the first compound is equal to or less than the LUMO energy level of the second compound, it is possible to prevent electrons from being trapped in the second compound and to reduce deterioration of the luminous materials, so that the OLED can increase its luminous lifespan. On the other hand, when the LUMO energy level LUMO1 of the first compound is higher than the LUMO energy level LUMO2 of the second compound, electrons are trapped in the LUMO energy level LUMO2 of the second compound, emission area is biased toward to HBL, ETL or cathode from the EML, the luminous material are deteriorated, and phenomena such as TTA (Triplet-Triplet Annihilation) and TPA (Two-photon Absorption) is occurred, and thereby reducing the luminous lifespan of the OLED.

The emission distribution may be measured as illustrated in FIG. 6. After designing the OLED structure as illustrated in FIG. 2, partitioning the EML into four areas (area I, area II, area III and area IV), and then dope each of the four areas with phosphorescent material. With the doping of the phosphorescent material and measuring the emission intensity, emission intensity of the luminous materials in each area can be measured. FIG. 5 is illustrates the emission intensity in each area using the phosphorescent material with red emission peak of about 614 nm in accordance with the following Example 1 (Ex. 1) and Comparative Example (Ref. 1).

With referring to FIG. 7, which is a schematic diagram illustrating a luminous mechanism among the luminous materials in an EML in accordance with an exemplary aspect of the present disclosure, each of exciton energies of the singlet energy level S₁^{H} and the triplet energy level T₁^{H} generated in the third compound H of host are transferred to the singlet energy level S₁^{DF} and the triplet energy level T₁^{DF} of the first compound DF of the delayed fluorescent material, respectively. Since the first compound DF has very narrow energy level bandgap ΔE_{ST}^{DF} between the singlet level S₁^{DF} and the triplet T₁^{DF}, the excitons at the triplet energy level T₁^{DF} are converted upwardly to the excitons at the singlet energy level S₁^{DF} in the first compound DF by RISC mechanism. For example, the energy level bandgap ΔE_{ST}^{DF} between the singlet energy level S₁^{DF} and the triplet energy level T₁^{DF} of the first compound may be equal to or less than about 0.3 eV. The exciton energy at the singlet energy level S₁^{DF} of the first compound is transferred to the singlet energy level S₁^{FD} of the second compound in which ultimate emission is occurred.

As described above, the first compound DF with the delayed fluorescence property has high quantum efficiency, while it shows bad color purity owing to its wide FWHM. On the contrary, the second compound FD with the fluorescence property has narrow FWHM, while it has low luminous efficiency because its triplet excitons are not involved in the luminous process.

However, in the OLED D1, exciton energies of the singlet energy level as well as the triplet energy level of the first compound as the delayed fluorescent material are transferred to the second compound of the fluorescent material, and then the second compound emits light ultimately. In addition, since the LUMO energy level LUMO1 of the first compound is equal to or deeper than the LUMO energy level LUMO2 of the second compound, it is possible to prevent electrons from being trapped in the second compound and to reduce the deterioration of the luminous materials. Therefore, the OLED D1 can improve its luminous lifespan and color purity. For example, the EML 240 includes the first compound having the Formulae 1 to 3 and the second compound having the Formulae 4 to 5 so that the OLED D1 can enhance significantly its luminous lifespan.

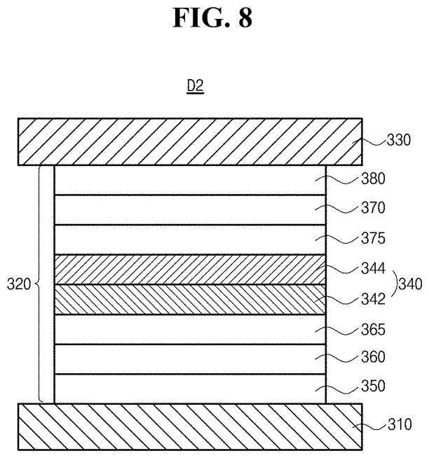

FIG. 8 is a schematic cross-sectional view illustrating an OLED in accordance with another exemplary aspect of the present disclosure. As illustrated in FIG. 8, the OLED D2 in accordance with the second aspect of the present disclosure include first and second electrodes 310 and 330 facing each other and an emissive layer 320 disposed between the first and second electrodes 310 and 330. The first electrode 310 may be an anode and the second electrode 330 may be a cathode.

The emissive layer 320 includes an EML 340. The emissive layer 320 may further comprise at least one of an HTL 360 disposed between the first electrode 310 and the EML 340 and an ETL 370 disposed between the second electrode 330 and the EML 340. Also, the emissive layer 320 may further comprise at least one of an HIL 350 disposed between the first electrode 310 and the HTL 360 and an EIL 380 disposed between the ETL 370 and the second electrode 330. In addition, the emissive layer 320 may further comprise at least one of an EBL 365 disposed between the EML 340 and the HTL 360 and an HBL 375 disposed between the EML 340 and the ETL 370.

The EML 340 comprises a first EML (EML1) 342 and a second EML (EML2) 344 each of which may be laminated sequentially. In one exemplary aspect, the EML2 344 may be disposed between the EML1 342 and the second electrode 330. Alternatively, the EML2 344 may be disposed between the EML1 342 and the first electrode.

One of the EML1 342 and the EML2 344 includes the first compound of the delayed fluorescent material having the Formulae 1 to 3 and the other of the EML1 342 and the EML2 344 includes the second compound of the fluorescent material having the Formulae 4 to 5. In addition, each of the EML1 342 and the EML2 344 may further include a fourth compound of a first host and a fifth compound of a second host, respectively. The fourth compound in the EML1 342 may be identical to or different from the fifth compound in the EML2 344. For example, each of the fourth compound in the EML1 342 and the fifth compound in the EML2 344 may be independently the third compound, as described above. Hereinafter, the OLED D2 where the EML2 342 includes the first compound will be described.

As described above, the first compound having the delayed fluorescent property has high quantum efficiency, but its color purity is not good owing to its wide FWHM. On the other hand, the second compound having the fluorescent property has narrow FWHM, but has lower luminous efficiency because its triplet exciton energy cannot participate in the luminous process. In addition, when the LUMO energy level relationship between the luminous materials are properly adjusted, for example, when the LUMO energy level LUMO1 of the first compound having the delayed fluorescent property is shallower than the LUMO energy level LUMO2 of the second compound having the fluorescent property, the luminous lifespan property of the OLED is decreased.

In the OLED D2, the triplet exciton energy of the first compound in the EML1 342 is converted upwardly its own singlet exciton energy by RISC mechanism, the singlet exciton energy of the first compound is transferred to the second compound in the EML2 344, and the second compound emits light ultimately. Since both the excitons of the singlet energy level and the excitons of the triplet energy level among the luminous materials can participate in the luminous process, the OLED D2 can enhance its luminous efficiency and implement excellent color purity because the ultimate emission is occurred at the second compound of the fluorescent material having narrow FWHM.

In addition, the LUMO energy level LUMO1 of the first compound is identical to or deeper than the LUMO energy level LUMO2 of the second compound, the luminous lifespan of the OLED D2 can be improved significantly, as described above.

In the EML1 342, the contents of the fourth compound may be identical to or larger than the contents of the first compound. In the EML2 344, the contents of the fifth compound may be identical to or larger than the contents of the second compound.

In addition, the contents of the first compound in the EML1 342 may be larger than the contents of the second compound in the EML2 344. In this case, exciton energy is efficiently transferred from the first compound in the EML1 342 to the second compound in the EML2 344 via FRET mechanism. As an example, the EML1 342 may comprise the first compound between about 1 wt % and about 50 wt %, for example, about 10 wt % and about 40 wt % such as about 20 wt % and about 40 wt %. The EML2 344 may comprise the second compound between about 1 wt % and about 10 wt %, for example, about 1 wt % and 5 wt %.

When the HBL 375 is disposed between the EML2 344 and the ETL 370, the fifth compound of the second host in the EML2 344 may be the same material as the HBL 375. In this case, the EML2 344 may have a hole blocking function as well as an emission function. In other words, the EML2 344 can act as a buffer layer for blocking holes. In one aspect, the HBL 375 may be omitted where the EML2 344 may be a hole blocking layer as well as an emitting material layer.

In another aspect, when the EML1 342 includes the second compound of the fluorescent material and the EBL 365 is disposed between the HTL 360 and the EML1 342, the hots in the EML1 342 may be the same as the EBL 365. In this case, the EML1 342 may have an electron blocking function as well as an emission function. In other words, the EML1 342 can act as a buffer layer for blocking electrons. In one aspect, the EBL 365 may be omitted where the EML1 342 may be an electron blocking layer as well as an emitting material layer.

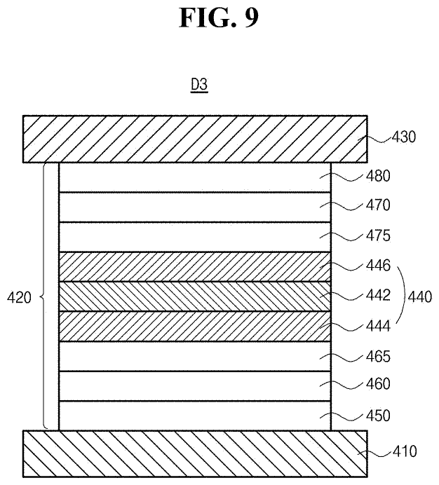

FIG. 9 is a schematic cross-sectional view illustrating an OLED having a triple-layered EML in accordance with another exemplary aspect of the present disclosure. As illustrated in FIG. 9, the OLED D3 in accordance with the third aspect of the present disclosure include first and second electrodes 410 and 430 facing each other and an emissive layer 420 disposed between the first and second electrodes 410 and 430. The first electrode 410 may be an anode and the second electrode 430 may be a cathode.

The emissive layer 420 includes an EML 440. The emissive layer 420 may further comprise at least one of an HTL 460 disposed between the first electrode 410 and the EML 440 and an ETL 470 disposed between the second electrode 430 and the EML 440. Also, the emissive layer 420 may further comprise at least one of an HIL 450 disposed between the first electrode 410 and the HTL 460 and an EIL 480 disposed between the ETL 470 and the second electrode 430. In addition, the emissive layer 420 may further comprise at least one of an EBL 465 disposed between the EML 440 and the HTL 460 and an HBL 475 disposed between the EML 440 and the ETL 470.

The EML 340 comprises a first EML (EML1) 442, a second EML (EML2) 444 disposed between the EML1 442 and the first electrode 410 and a third EML (EML3) 446 disposed between the EML1 442 and the second electrode 430. Namely, the EML 440 has a triple-layered structure of laminating sequentially the EML2 444, the EML1 442 and the EML3 446. For example, the EML1 442 may be disposed between the EBL 465 and the HBL 475, the EML2 444 may be disposed between the EBL 465 and the EML1 442 and the EML3 446 may be disposed between the HBL 475 and the EML1 442.

The EML1 442 includes the first compound of the delayed fluorescent material having the Formulae 1 to 3, each of the EML2 444 and the EML3 446 includes the second compound of the fluorescent material having the Formulae 4 to 5, respectively. The second compound in the EML2 444 may be identical to or different from the second compound in the EML3 446. In addition, each of the EML1 442, the EML2 444 and the EML3 446 may further include a fourth compound of a first host, a fifth compound of a second host or a sixth compound of a third host, respectively. The fourth to sixth compounds may be identical to or different form each other. For example, each of the fourth to sixth compound may be independently the third compound, as described.

In the OLED D3, the triplet exciton energy of the first compound in the EML1 442 is converted upwardly its own singlet exciton energy by RISC mechanism, the singlet exciton energy of the first compound is transferred to the second compounds in the EML2 444 and the EML3 446, and each of the second compounds emits light ultimately. Since both the excitons of the singlet energy level and the excitons of the triplet energy level among the luminous materials can participate in the luminous process, the OLED D3 can enhance its luminous efficiency and implement excellent color purity because the ultimate emission is occurred at the second compound of the fluorescent material having narrow FWHM.

In addition, the LUMO energy level LUMO1 of the first compound is identical to or deeper than the LUMO energy level LUMO2 of the second compound, the luminous lifespan of the OLED D3 can be improved significantly, as described above.

In the EML1 442, the contents of the fourth compound may be identical to or larger than the contents of the first compound. In the EML2 444, the contents of the fifth compound may be identical to or larger than the contents of the second compound. In addition, in the EML3 446, the contents of the sixth compound may be identical to or larger than the contents of the second compound.

In addition, the contents of the first compound in the EML1 442 may be larger than the contents of the second compound in the EML2 444 and the contents of the second compound in the EML3 446. In this case, exciton energy is efficiently transferred from the first compound in the EML1 442 to the second compound in the EML2 444 and to the second compound in the EML3 446 via FRET mechanism.

As an example, the EML1 442 may comprise the first compound between about 1 wt % and about 50 wt %, for example, about 10 wt % and about 40 wt % such as about 20 wt % and about 40 wt %. Each of the EML2 444 and the EML3 446 may comprise the second compound between about 1 wt % and about 10 wt %, for example, about 1 wt % and 5 wt %.

The fifth compound of the host in the EML2 444 may be the same material as the EBL 465. In this case, the EML2 444 may have an electron blocking function as well as an emission function. In other words, the EML2 444 can act as a buffer layer for blocking electrons. In one aspect, the EBL 465 may be omitted where the EML2 444 may be an electron blocking layer as well as an emitting material layer.

In another aspect, the sixth compound of the third host in the EML3 446 may be the same as the HBL 475. In this case, the EML3 446 may have a hole blocking function as well as an emission function. In other words, the EML3 446 can act as a buffer layer for blocking holes. In one aspect, the HBL 475 may be omitted where the EML3 446 may be a hole blocking layer as well as an emitting material layer.

In still another exemplary aspect, the fifth compound in the EML2 444 may be the same material as the EBL 465 and the sixth compound in the EML3 446 may be the same material as the HBL 475. In this aspect, the EML2 444 may have an electron blocking function as well as an emission function, and the EML3 446 may have a hole blocking function as well as an emission function. In other words, each of the EML2 444 and the EML3 446 can act as a buffer layer for blocking electrons or hole, respectively. In one aspect, the EBL 465 and the HBL 475 may be omitted where the EML2 444 may be an electron blocking layer as well as an emitting material layer and the EML3 446 may be a hole blocking layer as well as an emitting material layer.

### Example 1 (Ex. 1): Fabrication of OLED

An OLED having the following components was fabricated.

An anode (ITO, 50 nm); a HIL (HAT-CN of the following Formula 6-1, 7 nm); a HTL (NPB of the following Formula 6-2, 78 nm); an EBL (TAPC of the following Formula 6-3, 15 nm), an EML (m-CBP of the following Formula 6-6 (64 wt %) as the third compound, Compound 1-4 of Formula 3 (35 wt %) as the first compound, Compound 2-47 (1 wt %) as the second compound, 35 nm); a HBL (B3PYMPM of the following Formula 6-4, 10 nm); an ETL (TPBi of the following Formula 6-5, 25 nm), an EIL (LiF); and a cathode (Al).

### Examples 2-3 (Ex. 2-3): Fabrication of OLED

An OLED was fabricated using the same materials as Example 1, except that Compound 1-3 of Formula 3 (Example 2) or Compound 1-2 of Formula 3 (Example 3) was used as the first compound in the EML instead of the Compound 1-4.

### Comparative Examples 1-3 (Ref 1-3): Fabrication of OLED

An OLED was fabricated using the same materials as Example 1, except that Compound 7-1 of the following Formula 7-1 (Ref 1), Compound 7-2 of the following Formula 7-2 (Ref. 2) or Compound 7-3 of the following Formula 7-3 (Ref. 3) was used as the first compound in the EML instead of the Compound 1-4.

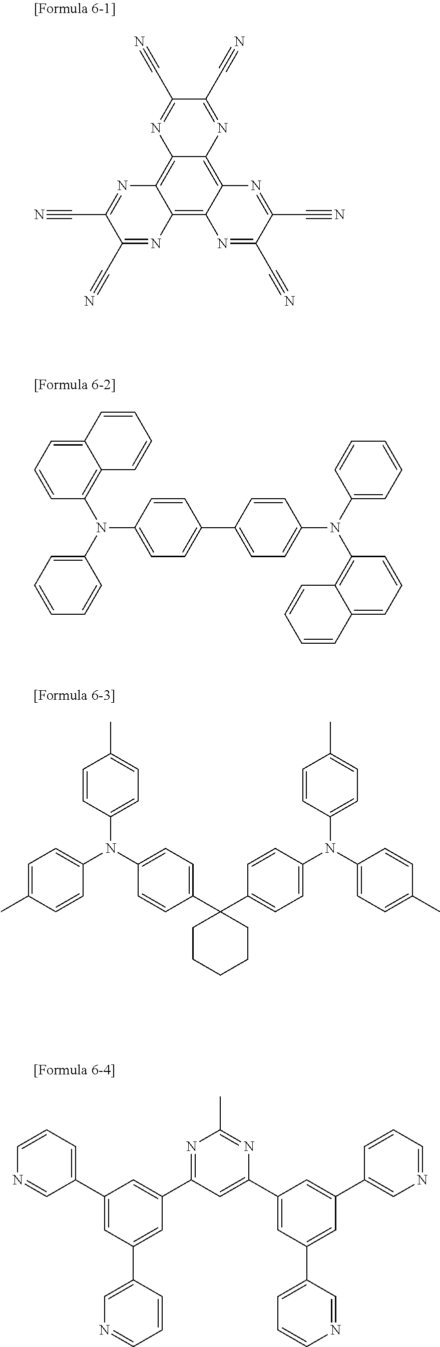

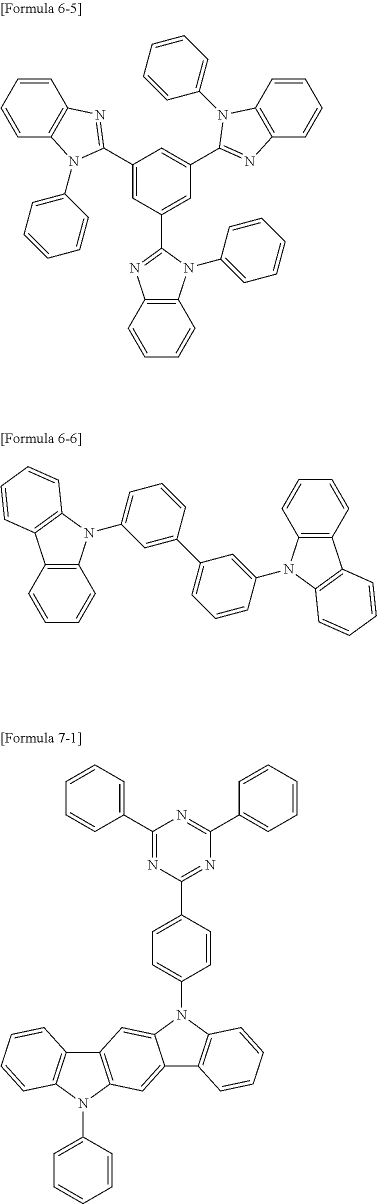 [SMILES: c1ccc(-n2c(-c3cc(-c4nc5ccccc5n4-c4ccccc4)cc(-c4nc5ccccc5n4-c4ccccc4)c3)nc3ccccc32)cc1 ; c1cc(-c2cccc(-n3c4ccccc4c4ccccc43)c2)cc(-n2c3ccccc3c3ccccc32)c1 ; c1ccc(-c2nc(-c3ccccc3)nc(-c3ccc(-n4c5ccccc5c5cc6c(cc54)c4ccccc4n6-c4ccccc4)cc3)n2)cc1]

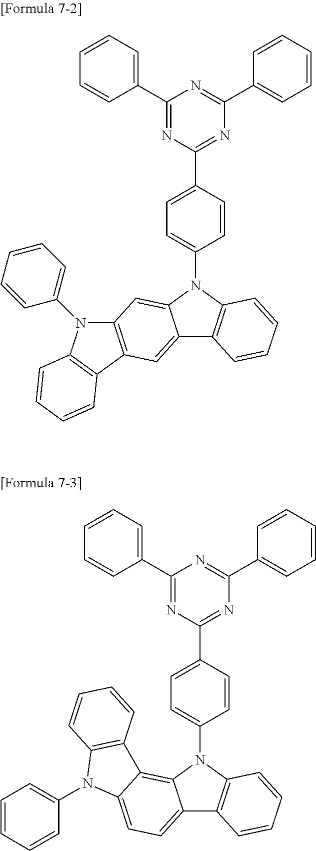 [SMILES: c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2nc(-c3ccccc3)nc(-c3ccc(-n4c5ccccc5c5ccc6c(c7ccccc7n6-c6ccccc6)c54)cc3)n2)cc1]

### Experimental Example 1: Measurement of Luminous Properties of OLED

Each of the OLEDs fabricated by Ex. 1-3 and Ref. 1-3 was connected to an external power source and then luminous properties, such as driving voltage (V), current efficiency (cd/A), maximum electro-luminescence wavelength (λₘₐₓ) and luminous lifespan (LT₉₅) for all the diodes were evaluated. The measurement results are shown in the following Table 1.

TABLE 1
Luminous Properties of OLED

| Sample | V | cd/A | λₘₐₓ | LT₉₅ |
|---|---|---|---|---|
| Ref. 1 | 3.6 | 46.9 | 554 | 190 |
| Ref. 2 | 3.6 | 44.2 | 554 | 130 |
| Ref. 3 | 3.5 | 43.2 | 554 | 120 |
| Ex. 1 | 3.8 | 45.3 | 558 | 600 |
| Ex. 2 | 3.7 | 46.1 | 556 | 450 |
| Ex. 3 | 3.7 | 43.0 | 556 | 350 |

As indicated in Table 1, compared to the OLEDs fabricated in the Comparative Examples, the OLEDs fabricated in the Examples showed improved luminous properties. Particularly, the OLEDs having the first compound corresponding to Formulae 1 to 3 and the second compound corresponding to Formulae 4 to 5 in the EML improved their luminous lifespan between 1.8 times up to about 5 times compared to the OLEDs fabricated in the Comparative Examples, with maintaining their driving voltages, current efficiency and maximum electro-luminescence peaks.

### Experimental Example 2: Measurement of Energy Level

The HOMO energy levels and LUMO energy levels of the first compounds such as Compound 1-4, Compound 1-3, Compound 1-2, Compound 7-1, Compound 7-2 and Compound 7-3 as well as the second compound such as Compound 2-47 were Measured. Measurement results are shown in the following Table 2.

TABLE 2
HOMO and LUMO Energy Level of Compounds

| Compound | LUMO (eV) | HOMO (eV) |
|---|---|---|
| 2-47 | −3.0 | −5.3 |
| 1-4 | −3.1 | −5.7 |
| 1-3 | −3.1 | −5.7 |
| 1-2 | −3.1 | −5.7 |
| D 7-1 | −2.8 | −5.8 |
| 7-2 | −2.8 | −5.8 |
| 7-3 | −2.9 | −5.8 |

As illustrated in Table 2, the LUMO energy level of the first compound corresponding to Formulae 1 to 3 (Compound 1-2, Compound 1-3 and Compound 1-4) is lower than of the LUMO energy level of the second compound (Compound 2-47) and the energy level bandgap between the LUMO energy level of the first compound and the second compound is equal to or less than 0.2 eV, so that the OLEDs in Examples 1-3 improved significantly their luminous lifespan. On the other hand, the LUMO energy level of the first compound in the Comparative Examples (Compound 7-1, Compound 7-2 and Compound 7-3) is shallower than the second compound (Compound 2-6), so that the luminous properties in the OLED fabricated in the Comparative Examples are reduced.

It will be apparent to those skilled in the art that various modifications and variations can be made in the OLED and the organic light emitting device including the OLED of the present disclosure without departing from the technical idea or scope of the disclosure. Thus, it is intended that the present disclosure cover the modifications and variations of this disclosure provided they come within the scope of the appended claims and their equivalents.

What is claimed is:

1. An organic light emitting diode, comprising:
a first electrode;
a second electrode facing the first electrode; and
at least one emitting material layer disposed between the first and second electrodes,
wherein the at least one emitting material layer comprises a first compound and a second compound, and
wherein the first compound has the following structure of Formula 1 and the second compound has the following structure of Formula 4: 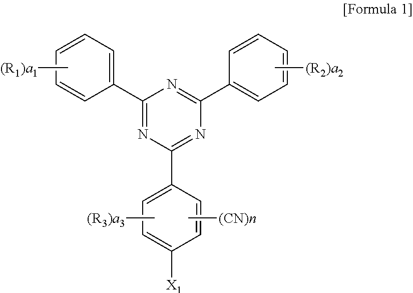
wherein each of R₁ to R₃ is independently selected from the group consisting of deuterium, tritium, a C₁-C₂₀ alkyl group, a C₆-C₃₀ aryl group and a C₃-C₄₀ hetero aryl group, or adjacent two of R₁ to R₃ form a fused ring; each of a₁ and a₂ is independently an integer of 0 to 5; a₃ is an integer of 0 to 3; n is an integer of 1 to 4; and X₁ is an unsubstituted or substituted hetero aromatic ring including at least one of N, O and S as a nuclear atom; 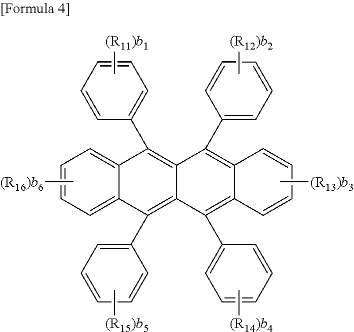
wherein each of R₁₁ to R₁₆ is independently selected from the group consisting of deuterium, tritium, a C₁-C₂₀ alkyl group, a C₆-C₃₀ aryl group and a C₃-C₄₀ hetero aryl group, or adjacent two of R₁₁ to R₁₆ form a fused ring; each of b₁, b₂, b₄ and b₅ is independently an integer of 0 to 5; and each of b₃ and b₆ is independently an integer of 0 to 4.

2. The organic light emitting diode of claim 1, wherein the first compound has the following structure of Formula 2: 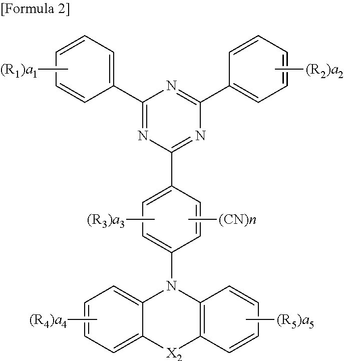
wherein each of R₁ to R₅ is independently selected from the group consisting of deuterium, tritium, a C₁-C₂₀ alkyl group, a C₆-C₃₀ aryl group and a C₃-C₄₀ hetero aryl group, or adjacent two of R₁ to R₅ form a fused ring which is unsubstituted or substituted with deuterium, tritium, a C₁-C₂₀ alkyl group, a C₆-C₃₀ aryl group or a C₃-C₄₀ hetero aryl group; each of a₁ and a₂ is independently an integer of 0 to 5; each of a₄ and as is independently an integer of 0 to 4; a₃ is an integer of 0 to 3; n is an integer of 1 to 4; and X₂ is a single bond, CR₆R₇, NR₆, O or S, wherein each of R₆ and R₇ is independently selected from the group consisting of protium, deuterium, tritium, a C₁-C₂₀ alkyl group, a C₆-C₃₀ aryl group and a C₃-C₄₀ hetero aryl group.

3. The organic light emitting diode of claim 1, wherein a lowest unoccupied molecular orbital energy level of the first compound is lower than or identical to a lowest unoccupied molecular orbital energy level of the second compound.

4. The organic light emitting diode of claim 1, wherein a lowest unoccupied molecular orbital energy level LUMO1 of the first compound and a lowest unoccupied molecular orbital energy level LUMO2 of the second compound satisfy the following relationship:
LUMO2−LUMO1≤0.2 eV.

5. The organic light emitting diode of claim 1, wherein the first compound has an energy level bandgap between about 2.0 eV and about 2.8 eV.

6. The organic light emitting diode of claim 1, wherein X₁ in Formula 1 includes a carbazolyl moiety.

7. The organic light emitting diode of claim 6, wherein the carbazolyl moiety includes an indeno-carbazolyl moiety, an indolo-carbazolyl moiety, a benzothieno-carbazolyl moiety and a benzofuro-carbazolyl moiety.

8. The organic light emitting diode of claim 1, wherein the first compound is selected from the following compounds having the structure of Formula 3:

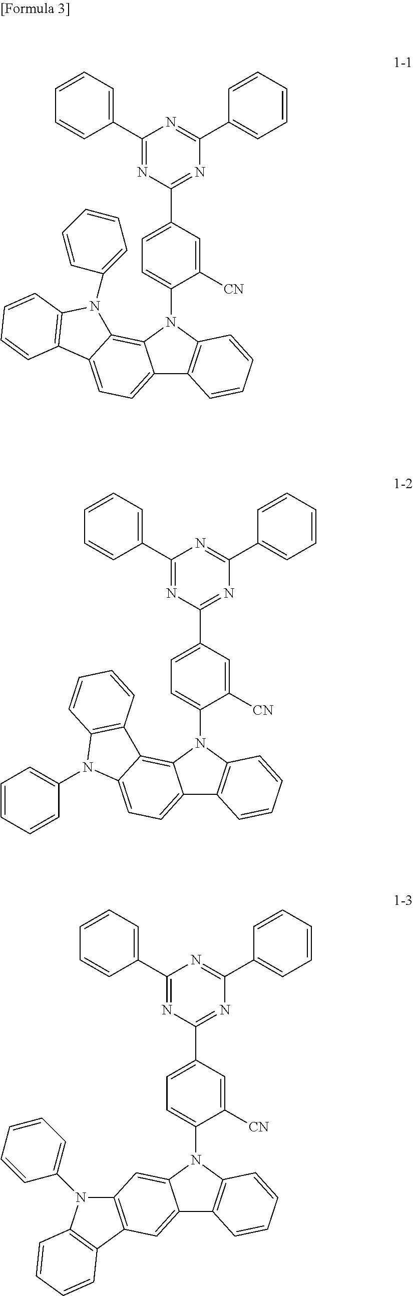 [SMILES: N#Cc1cc(-c2nc(-c3ccccc3)nc(-c3ccccc3)n2)ccc1-n1c2ccccc2c2ccc3c4ccccc4n(-c4ccccc4)c3c21 ; N#Cc1cc(-c2nc(-c3ccccc3)nc(-c3ccccc3)n2)ccc1-n1c2ccccc2c2ccc3c(c4ccccc4n3-c3ccccc3)c21 ; N#Cc1cc(-c2nc(-c3ccccc3)nc(-c3ccccc3)n2)ccc1-n1c2ccccc2c2cc3c4ccccc4n(-c4ccccc4)c3cc21]

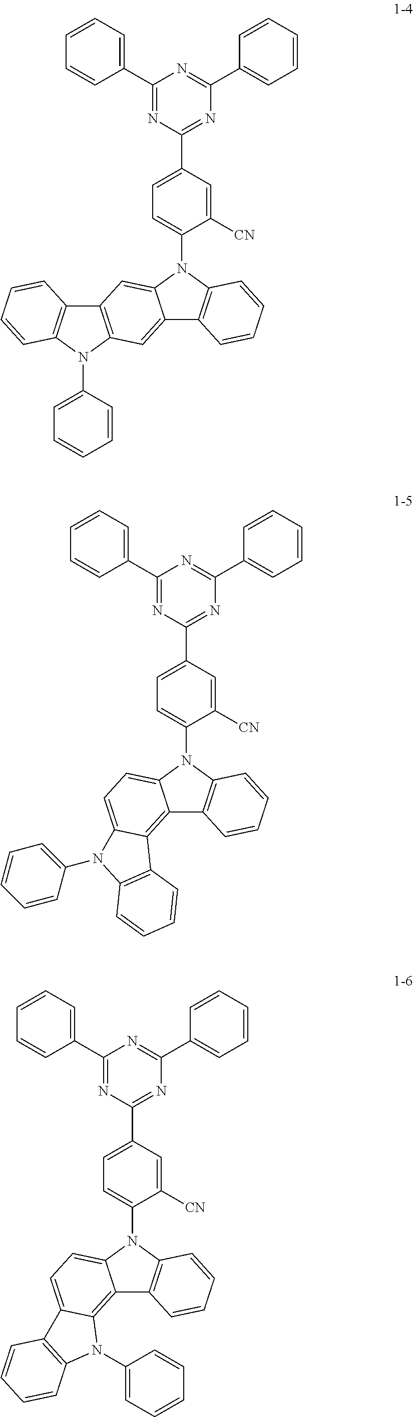 [SMILES: N#Cc1cc(-c2nc(-c3ccccc3)nc(-c3ccccc3)n2)ccc1-n1c2ccccc2c2cc3c(cc21)c1ccccc1n3-c1ccccc1 ; N#Cc1cc(-c2nc(-c3ccccc3)nc(-c3ccccc3)n2)ccc1-n1c2ccccc2c2c3c4ccccc4n(-c4ccccc4)c3ccc21 ; N#Cc1cc(-c2nc(-c3ccccc3)nc(-c3ccccc3)n2)ccc1-n1c2ccccc2c2c1ccc1c3ccccc3n(-c3ccccc3)c12]

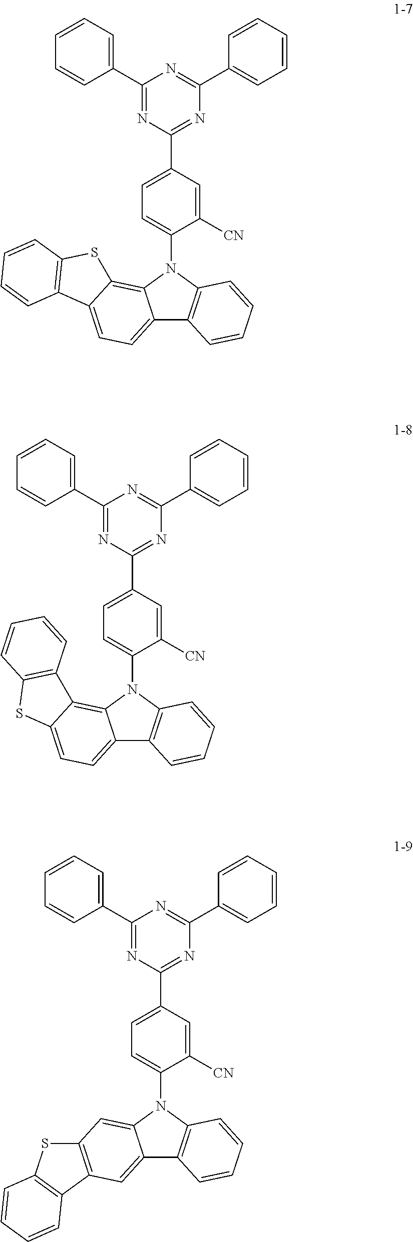 [SMILES: N#Cc1cc(-c2nc(-c3ccccc3)nc(-c3ccccc3)n2)ccc1-n1c2ccccc2c2ccc3c4ccccc4sc3c21 ; N#Cc1cc(-c2nc(-c3ccccc3)nc(-c3ccccc3)n2)ccc1-n1c2ccccc2c2ccc3sc4ccccc4c3c21 ; N#Cc1cc(-c2nc(-c3ccccc3)nc(-c3ccccc3)n2)ccc1-n1c2ccccc2c2cc3c(cc21)sc1ccccc13]

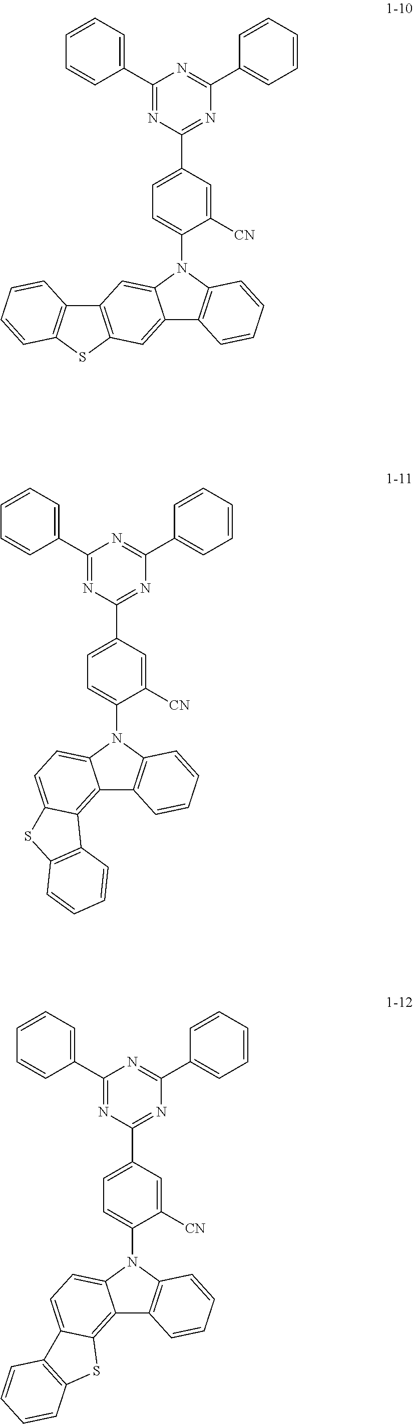 [SMILES: N#Cc1cc(-c2nc(-c3ccccc3)nc(-c3ccccc3)n2)ccc1-n1c2ccccc2c2cc3sc4ccccc4c3cc21 ; N#Cc1cc(-c2nc(-c3ccccc3)nc(-c3ccccc3)n2)ccc1-n1c2ccccc2c2c3c(ccc21)sc1ccccc13 ; N#Cc1cc(-c2nc(-c3ccccc3)nc(-c3ccccc3)n2)ccc1-n1c2ccccc2c2c3sc4ccccc4c3ccc21]

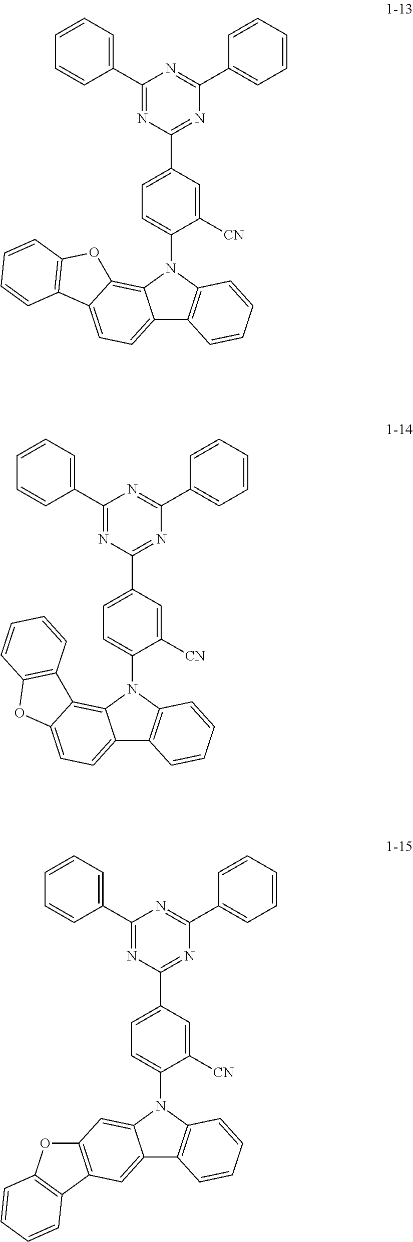 [SMILES: N#Cc1cc(-c2nc(-c3ccccc3)nc(-c3ccccc3)n2)ccc1-n1c2ccccc2c2ccc3c4ccccc4oc3c21 ; N#Cc1cc(-c2nc(-c3ccccc3)nc(-c3ccccc3)n2)ccc1-n1c2ccccc2c2ccc3oc4ccccc4c3c21 ; N#Cc1cc(-c2nc(-c3ccccc3)nc(-c3ccccc3)n2)ccc1-n1c2ccccc2c2cc3c(cc21)oc1ccccc13]

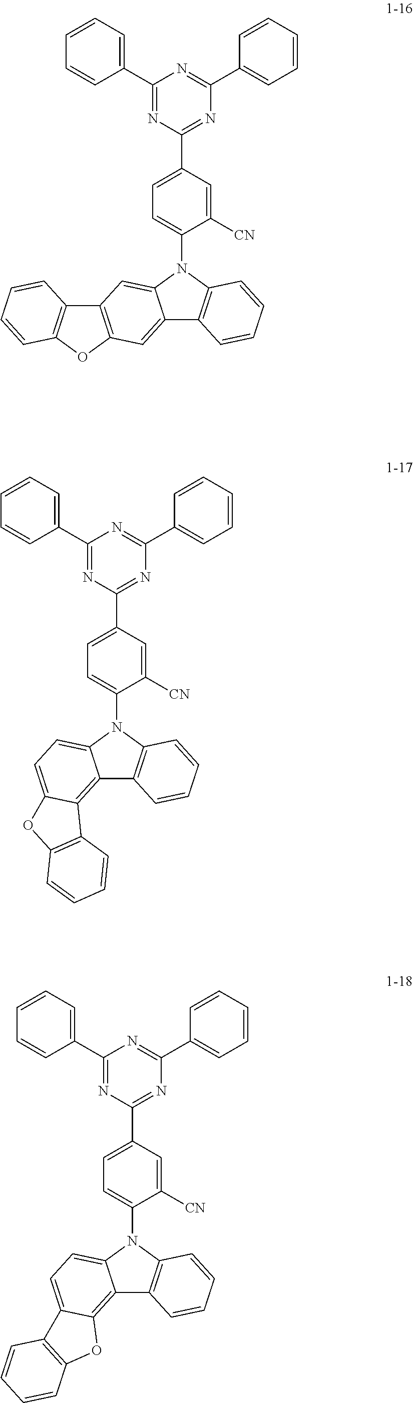 [SMILES: N#Cc1cc(-c2nc(-c3ccccc3)nc(-c3ccccc3)n2)ccc1-n1c2ccccc2c2cc3oc4ccccc4c3cc21 ; N#Cc1cc(-c2nc(-c3ccccc3)nc(-c3ccccc3)n2)ccc1-n1c2ccccc2c2c3c(ccc21)oc1ccccc13 ; N#Cc1cc(-c2nc(-c3ccccc3)nc(-c3ccccc3)n2)ccc1-n1c2ccccc2c2c3oc4ccccc4c3ccc21]

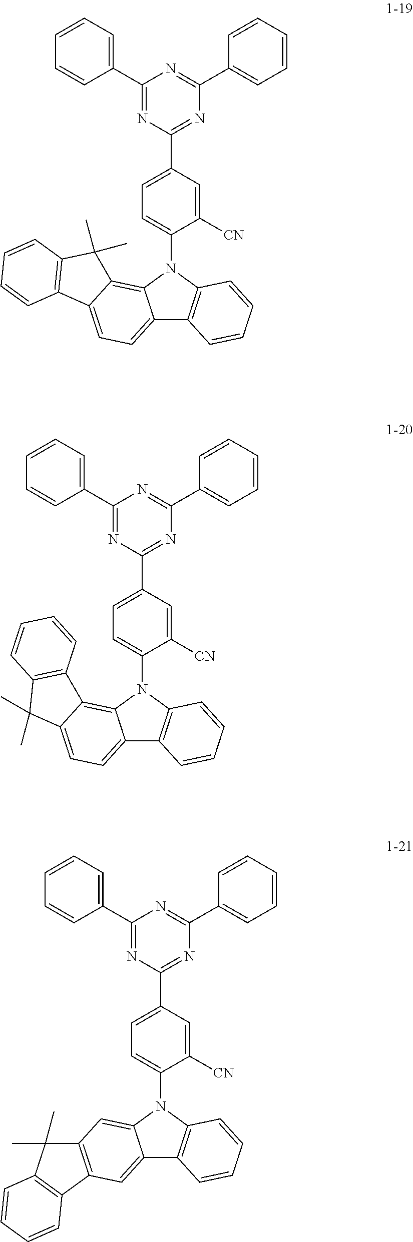 [SMILES: CC1(C)c2ccccc2-c2ccc3c4ccccc4n(-c4ccc(-c5nc(-c6ccccc6)nc(-c6ccccc6)n5)cc4C#N)c3c21 ; CC1(C)c2ccccc2-c2c1ccc1c3ccccc3n(-c3ccc(-c4nc(-c5ccccc5)nc(-c5ccccc5)n4)cc3C#N)c21 ; CC1(C)c2ccccc2-c2cc3c4ccccc4n(-c4ccc(-c5nc(-c6ccccc6)nc(-c6ccccc6)n5)cc4C#N)c3cc21]

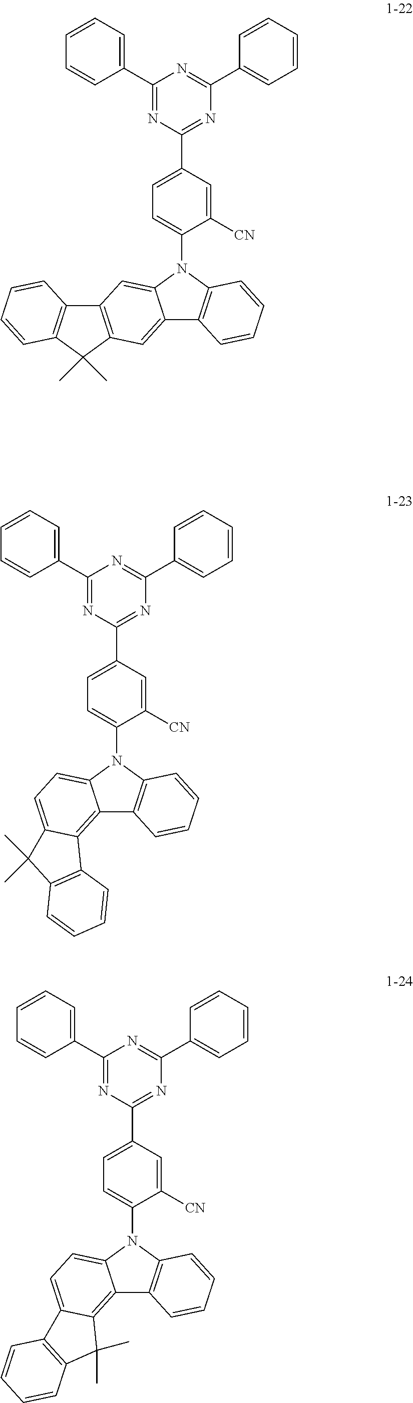 [SMILES: CC1(C)c2ccccc2-c2cc3c(cc21)c1ccccc1n3-c1ccc(-c2nc(-c3ccccc3)nc(-c3ccccc3)n2)cc1C#N ; CC1(C)c2ccccc2-c2c1ccc1c2c2ccccc2n1-c1ccc(-c2nc(-c3ccccc3)nc(-c3ccccc3)n2)cc1C#N ; CC1(C)c2ccccc2-c2ccc3c(c21)c1ccccc1n3-c1ccc(-c2nc(-c3ccccc3)nc(-c3ccccc3)n2)cc1C#N]

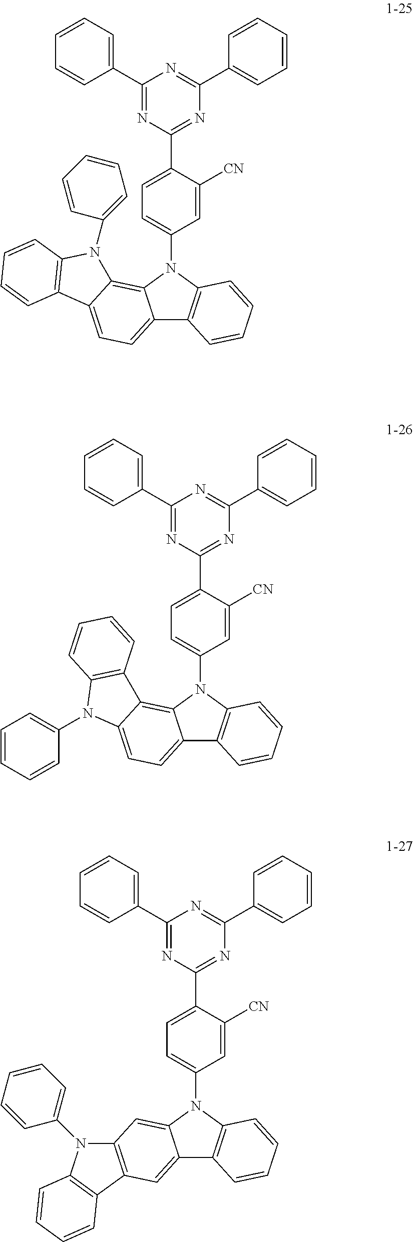 [SMILES: N#Cc1cc(-n2c3ccccc3c3ccc4c5ccccc5n(-c5ccccc5)c4c32)ccc1-c1nc(-c2ccccc2)nc(-c2ccccc2)n1 ; N#Cc1cc(-n2c3ccccc3c3ccc4c(c5ccccc5n4-c4ccccc4)c32)ccc1-c1nc(-c2ccccc2)nc(-c2ccccc2)n1 ; N#Cc1cc(-n2c3ccccc3c3cc4c5ccccc5n(-c5ccccc5)c4cc32)ccc1-c1nc(-c2ccccc2)nc(-c2ccccc2)n1]

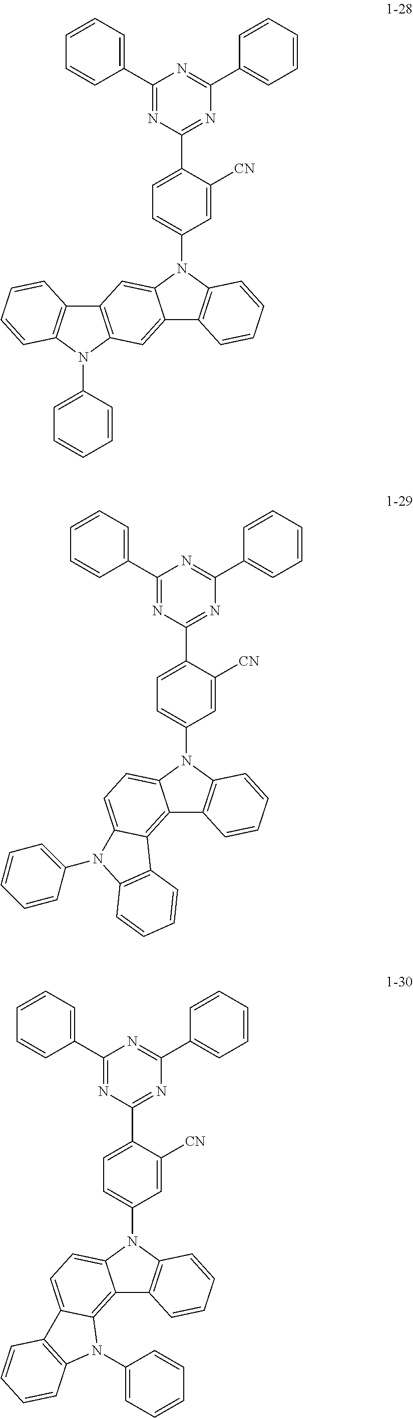 [SMILES: N#Cc1cc(-n2c3ccccc3c3cc4c(cc32)c2ccccc2n4-c2ccccc2)ccc1-c1nc(-c2ccccc2)nc(-c2ccccc2)n1 ; N#Cc1cc(-n2c3ccccc3c3c4c5ccccc5n(-c5ccccc5)c4ccc32)ccc1-c1nc(-c2ccccc2)nc(-c2ccccc2)n1 ; N#Cc1cc(-n2c3ccccc3c3c2ccc2c4ccccc4n(-c4ccccc4)c23)ccc1-c1nc(-c2ccccc2)nc(-c2ccccc2)n1]

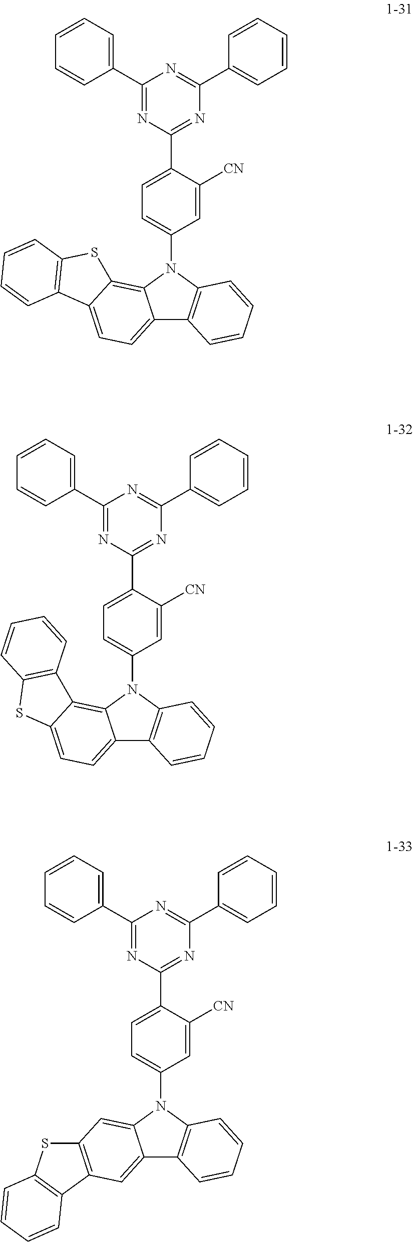 [SMILES: N#Cc1cc(-n2c3ccccc3c3ccc4c5ccccc5sc4c32)ccc1-c1nc(-c2ccccc2)nc(-c2ccccc2)n1 ; N#Cc1cc(-n2c3ccccc3c3ccc4sc5ccccc5c4c32)ccc1-c1nc(-c2ccccc2)nc(-c2ccccc2)n1 ; N#Cc1cc(-n2c3ccccc3c3cc4c(cc32)sc2ccccc24)ccc1-c1nc(-c2ccccc2)nc(-c2ccccc2)n1]

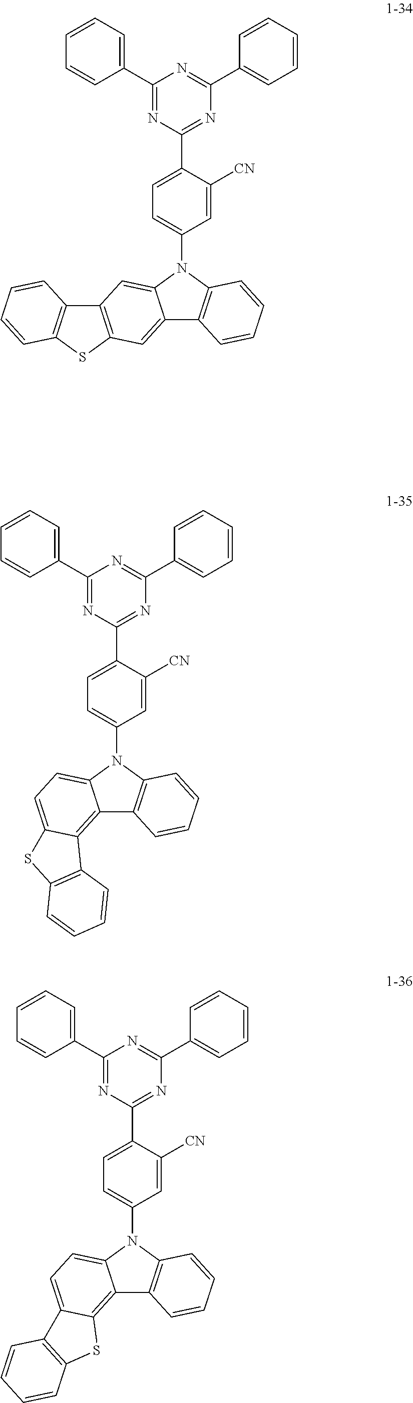 [SMILES: N#Cc1cc(-n2c3ccccc3c3cc4sc5ccccc5c4cc32)ccc1-c1nc(-c2ccccc2)nc(-c2ccccc2)n1 ; N#Cc1cc(-n2c3ccccc3c3c4c(ccc32)sc2ccccc24)ccc1-c1nc(-c2ccccc2)nc(-c2ccccc2)n1 ; N#Cc1cc(-n2c3ccccc3c3c4sc5ccccc5c4ccc32)ccc1-c1nc(-c2ccccc2)nc(-c2ccccc2)n1]

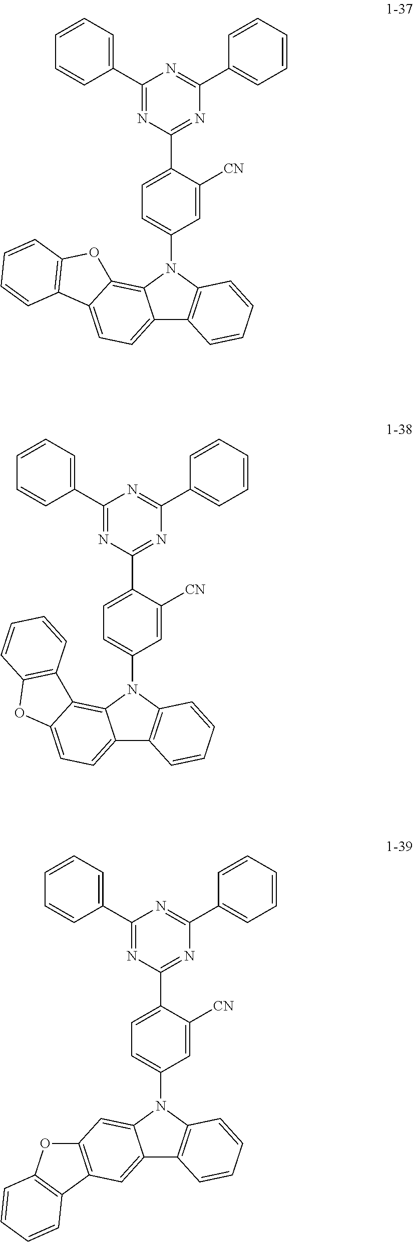 [SMILES: N#Cc1cc(-n2c3ccccc3c3ccc4c5ccccc5oc4c32)ccc1-c1nc(-c2ccccc2)nc(-c2ccccc2)n1 ; N#Cc1cc(-n2c3ccccc3c3ccc4oc5ccccc5c4c32)ccc1-c1nc(-c2ccccc2)nc(-c2ccccc2)n1 ; N#Cc1cc(-n2c3ccccc3c3cc4c(cc32)oc2ccccc24)ccc1-c1nc(-c2ccccc2)nc(-c2ccccc2)n1]

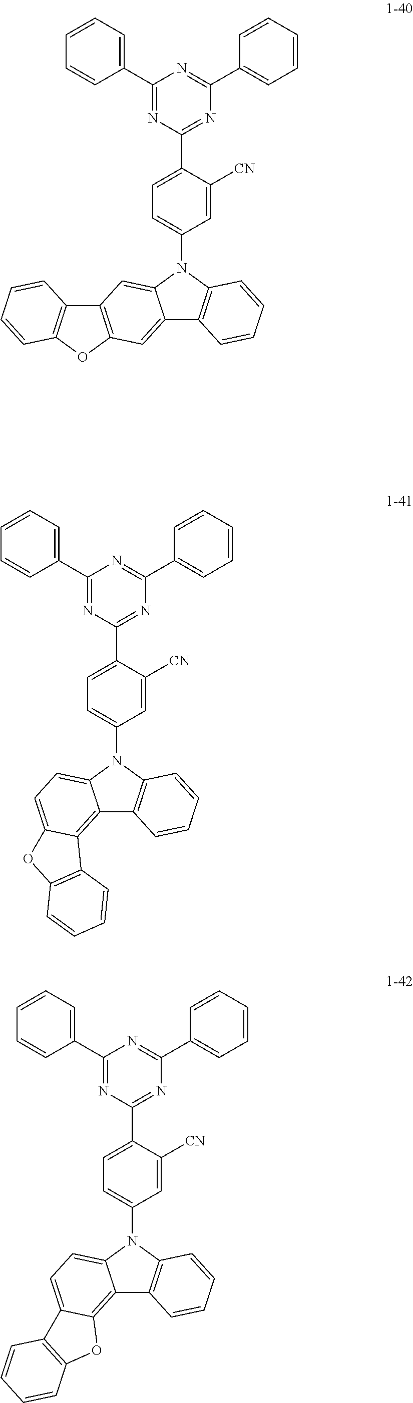 [SMILES: N#Cc1cc(-n2c3ccccc3c3cc4oc5ccccc5c4cc32)ccc1-c1nc(-c2ccccc2)nc(-c2ccccc2)n1 ; N#Cc1cc(-n2c3ccccc3c3c4c(ccc32)oc2ccccc24)ccc1-c1nc(-c2ccccc2)nc(-c2ccccc2)n1 ; N#Cc1cc(-n2c3ccccc3c3c4oc5ccccc5c4ccc32)ccc1-c1nc(-c2ccccc2)nc(-c2ccccc2)n1]

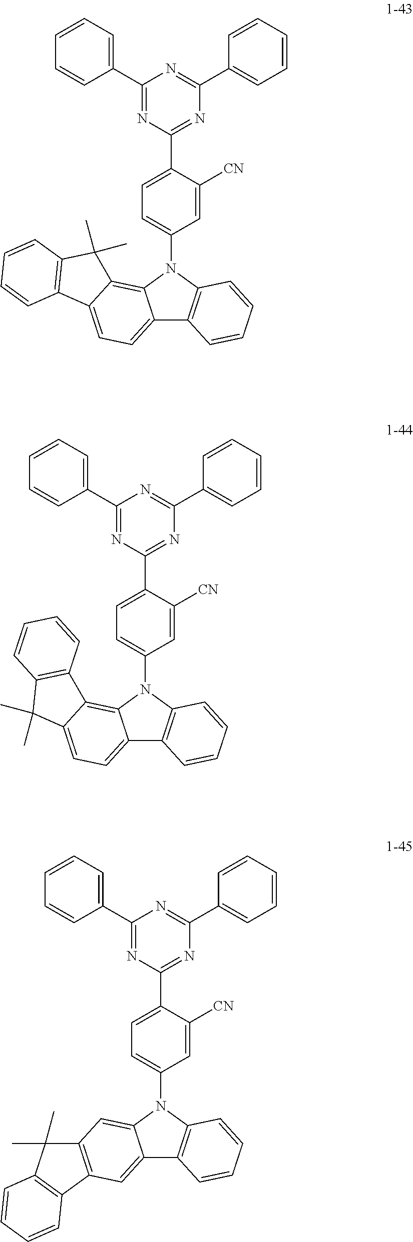 [SMILES: CC1(C)c2ccccc2-c2ccc3c4ccccc4n(-c4ccc(-c5nc(-c6ccccc6)nc(-c6ccccc6)n5)c(C#N)c4)c3c21 ; CC1(C)c2ccccc2-c2c1ccc1c3ccccc3n(-c3ccc(-c4nc(-c5ccccc5)nc(-c5ccccc5)n4)c(C#N)c3)c21 ; CC1(C)c2ccccc2-c2cc3c4ccccc4n(-c4ccc(-c5nc(-c6ccccc6)nc(-c6ccccc6)n5)c(C#N)c4)c3cc21]

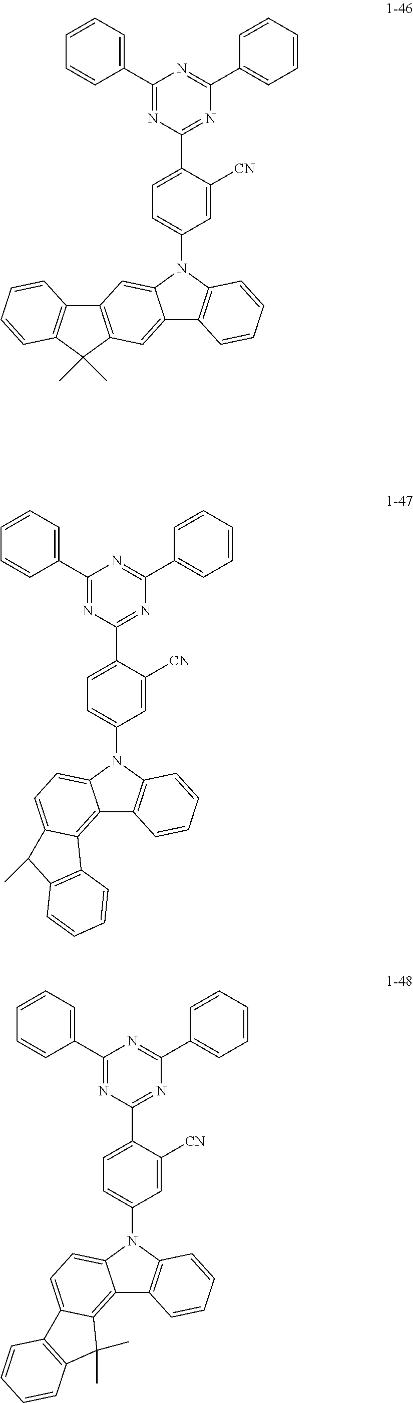 [SMILES: CC1(C)c2ccccc2-c2cc3c(cc21)c1ccccc1n3-c1ccc(-c2nc(-c3ccccc3)nc(-c3ccccc3)n2)c(C#N)c1 ; CC1c2ccccc2-c2c1ccc1c2c2ccccc2n1-c1ccc(-c2nc(-c3ccccc3)nc(-c3ccccc3)n2)c(C#N)c1 ; CC1(C)c2ccccc2-c2ccc3c(c21)c1ccccc1n3-c1ccc(-c2nc(-c3ccccc3)nc(-c3ccccc3)n2)c(C#N)c1]

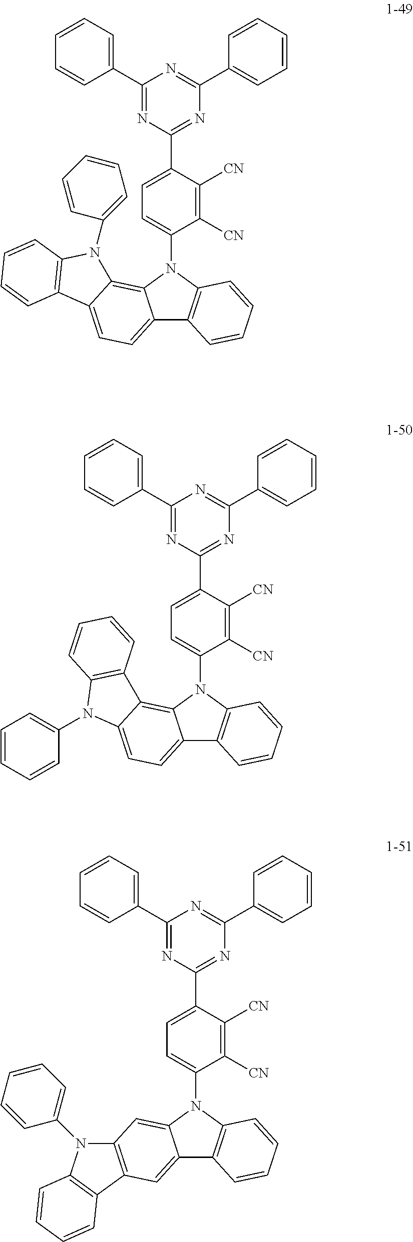 [SMILES: N#Cc1c(-c2nc(-c3ccccc3)nc(-c3ccccc3)n2)ccc(-n2c3ccccc3c3ccc4c5ccccc5n(-c5ccccc5)c4c32)c1C#N ; N#Cc1c(-c2nc(-c3ccccc3)nc(-c3ccccc3)n2)ccc(-n2c3ccccc3c3ccc4c(c5ccccc5n4-c4ccccc4)c32)c1C#N ; N#Cc1c(-c2nc(-c3ccccc3)nc(-c3ccccc3)n2)ccc(-n2c3ccccc3c3cc4c5ccccc5n(-c5ccccc5)c4cc32)c1C#N]

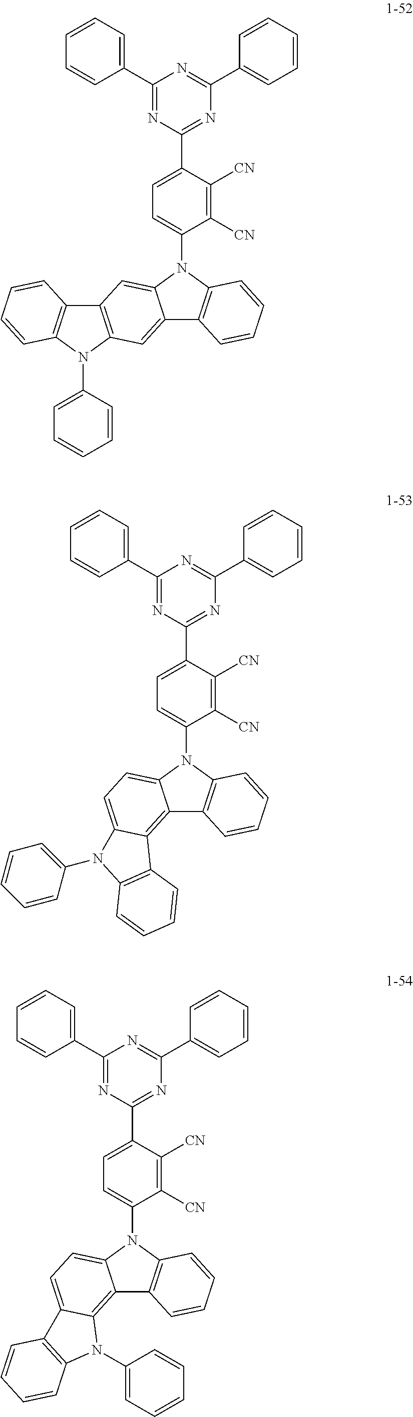 [SMILES: N#Cc1c(-c2nc(-c3ccccc3)nc(-c3ccccc3)n2)ccc(-n2c3ccccc3c3cc4c(cc32)c2ccccc2n4-c2ccccc2)c1C#N ; N#Cc1c(-c2nc(-c3ccccc3)nc(-c3ccccc3)n2)ccc(-n2c3ccccc3c3c4c5ccccc5n(-c5ccccc5)c4ccc32)c1C#N ; N#Cc1c(-c2nc(-c3ccccc3)nc(-c3ccccc3)n2)ccc(-n2c3ccccc3c3c2ccc2c4ccccc4n(-c4ccccc4)c23)c1C#N]

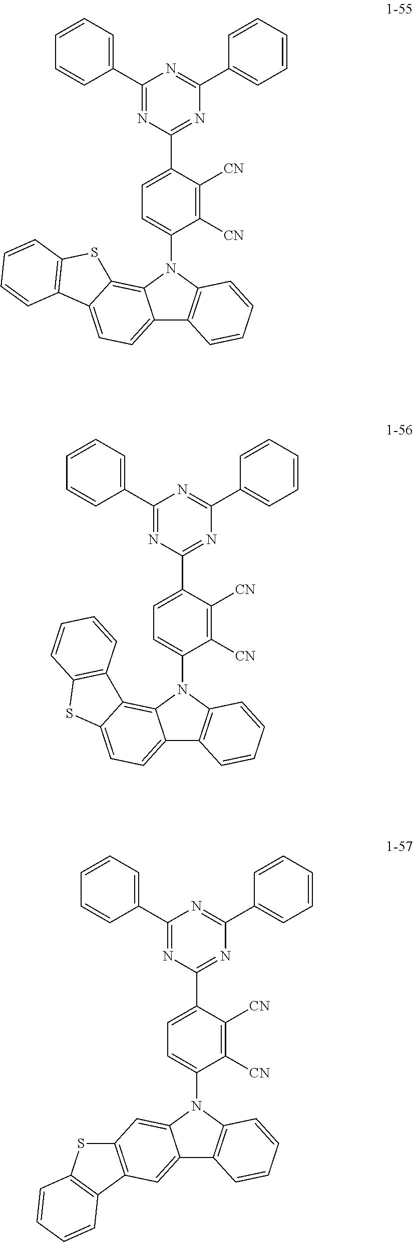 [SMILES: N#Cc1c(-c2nc(-c3ccccc3)nc(-c3ccccc3)n2)ccc(-n2c3ccccc3c3ccc4c5ccccc5sc4c32)c1C#N ; N#Cc1c(-c2nc(-c3ccccc3)nc(-c3ccccc3)n2)ccc(-n2c3ccccc3c3ccc4sc5ccccc5c4c32)c1C#N ; N#Cc1c(-c2nc(-c3ccccc3)nc(-c3ccccc3)n2)ccc(-n2c3ccccc3c3cc4c(cc32)sc2ccccc24)c1C#N]

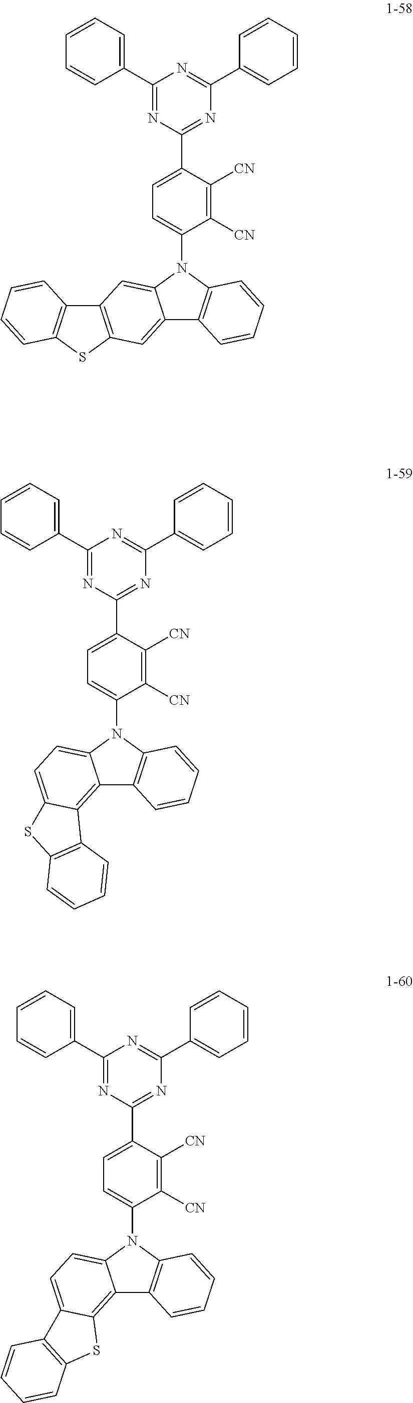 [SMILES: N#Cc1c(-c2nc(-c3ccccc3)nc(-c3ccccc3)n2)ccc(-n2c3ccccc3c3cc4sc5ccccc5c4cc32)c1C#N ; N#Cc1c(-c2nc(-c3ccccc3)nc(-c3ccccc3)n2)ccc(-n2c3ccccc3c3c4c(ccc32)sc2ccccc24)c1C#N ; N#Cc1c(-c2nc(-c3ccccc3)nc(-c3ccccc3)n2)ccc(-n2c3ccccc3c3c4sc5ccccc5c4ccc32)c1C#N]

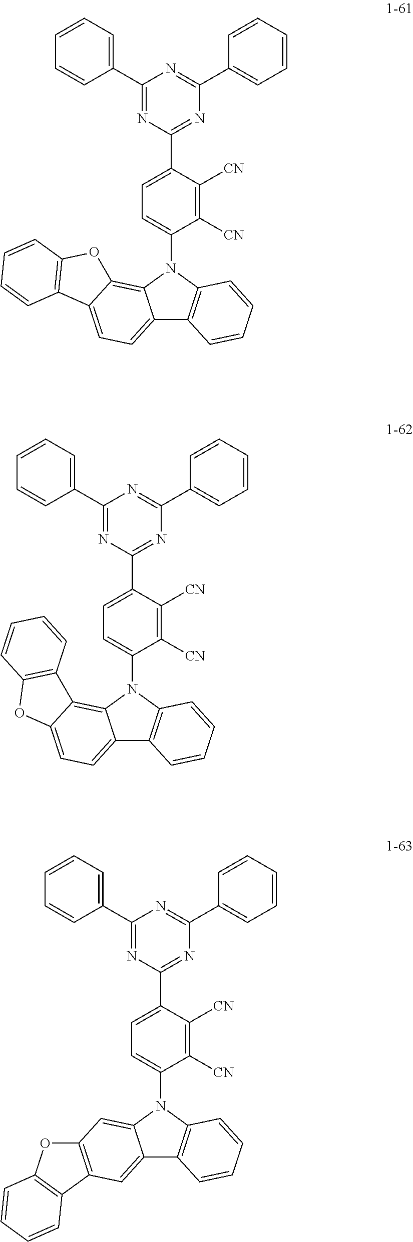 [SMILES: N#Cc1c(-c2nc(-c3ccccc3)nc(-c3ccccc3)n2)ccc(-n2c3ccccc3c3ccc4c5ccccc5oc4c32)c1C#N ; N#Cc1c(-c2nc(-c3ccccc3)nc(-c3ccccc3)n2)ccc(-n2c3ccccc3c3ccc4oc5ccccc5c4c32)c1C#N ; N#Cc1c(-c2nc(-c3ccccc3)nc(-c3ccccc3)n2)ccc(-n2c3ccccc3c3cc4c(cc32)oc2ccccc24)c1C#N]

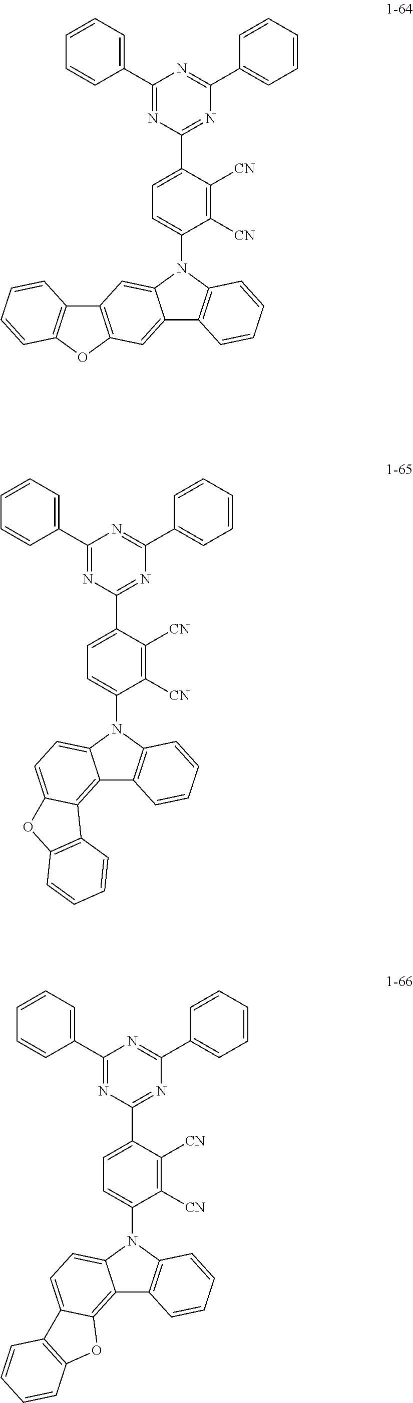 [SMILES: N#Cc1c(-c2nc(-c3ccccc3)nc(-c3ccccc3)n2)ccc(-n2c3ccccc3c3cc4oc5ccccc5c4cc32)c1C#N ; N#Cc1c(-c2nc(-c3ccccc3)nc(-c3ccccc3)n2)ccc(-n2c3ccccc3c3c4c(ccc32)oc2ccccc24)c1C#N ; N#Cc1c(-c2nc(-c3ccccc3)nc(-c3ccccc3)n2)ccc(-n2c3ccccc3c3c4oc5ccccc5c4ccc32)c1C#N]

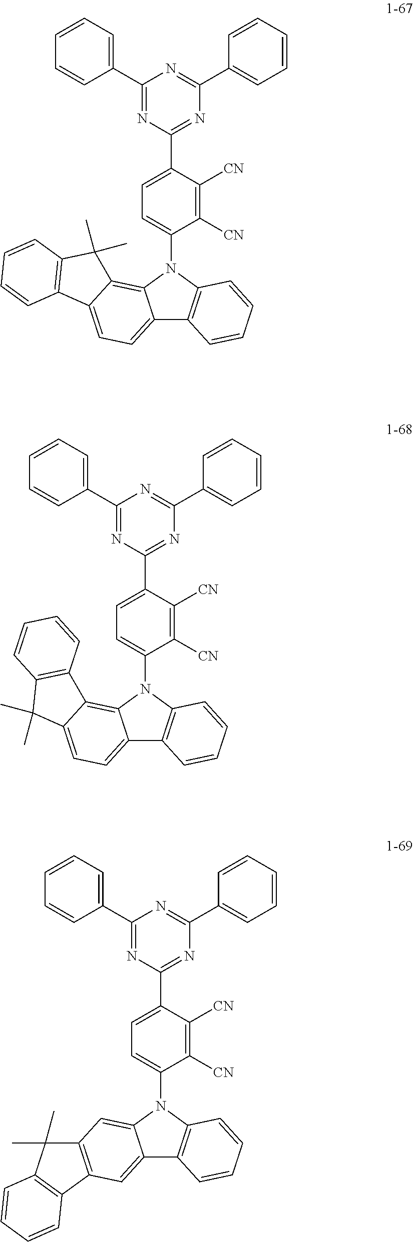 [SMILES: CC1(C)c2ccccc2-c2ccc3c4ccccc4n(-c4ccc(-c5nc(-c6ccccc6)nc(-c6ccccc6)n5)c(C#N)c4C#N)c3c21 ; CC1(C)c2ccccc2-c2c1ccc1c3ccccc3n(-c3ccc(-c4nc(-c5ccccc5)nc(-c5ccccc5)n4)c(C#N)c3C#N)c21 ; CC1(C)c2ccccc2-c2cc3c4ccccc4n(-c4ccc(-c5nc(-c6ccccc6)nc(-c6ccccc6)n5)c(C#N)c4C#N)c3cc21]

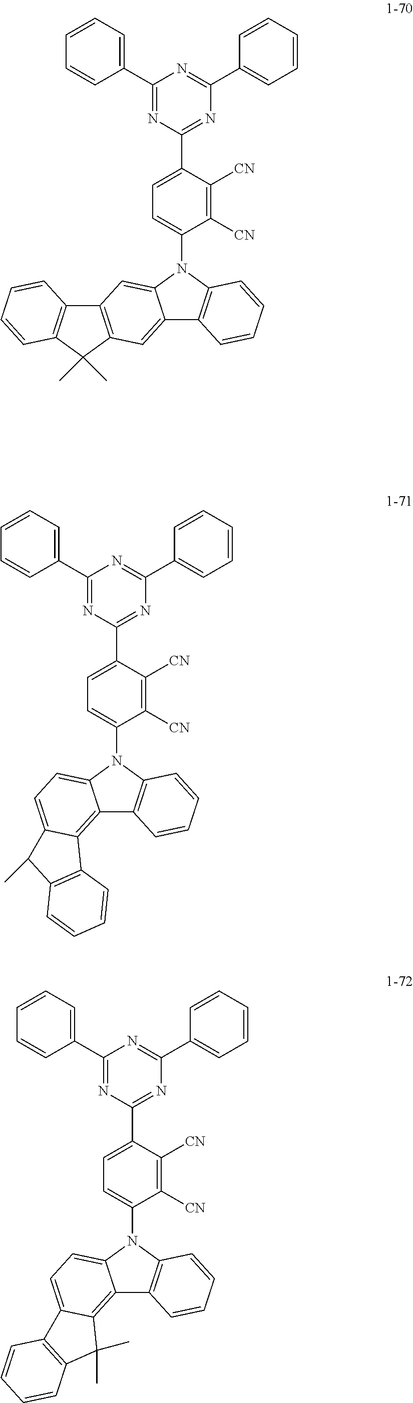 [SMILES: CC1(C)c2ccccc2-c2cc3c(cc21)c1ccccc1n3-c1ccc(-c2nc(-c3ccccc3)nc(-c3ccccc3)n2)c(C#N)c1C#N ; CC1c2ccccc2-c2c1ccc1c2c2ccccc2n1-c1ccc(-c2nc(-c3ccccc3)nc(-c3ccccc3)n2)c(C#N)c1C#N ; CC1(C)c2ccccc2-c2ccc3c(c21)c1ccccc1n3-c1ccc(-c2nc(-c3ccccc3)nc(-c3ccccc3)n2)c(C#N)c1C#N]

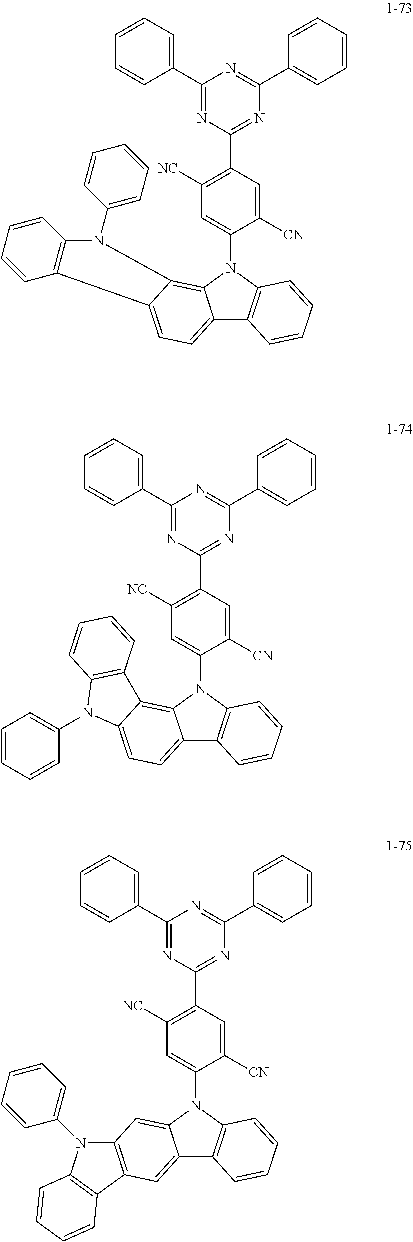 [SMILES: N#Cc1cc(-n2c3ccccc3c3ccc4c5ccccc5n(-c5ccccc5)c4c32)c(C#N)cc1-c1nc(-c2ccccc2)nc(-c2ccccc2)n1 ; N#Cc1cc(-n2c3ccccc3c3ccc4c(c5ccccc5n4-c4ccccc4)c32)c(C#N)cc1-c1nc(-c2ccccc2)nc(-c2ccccc2)n1 ; N#Cc1cc(-n2c3ccccc3c3cc4c5ccccc5n(-c5ccccc5)c4cc32)c(C#N)cc1-c1nc(-c2ccccc2)nc(-c2ccccc2)n1]

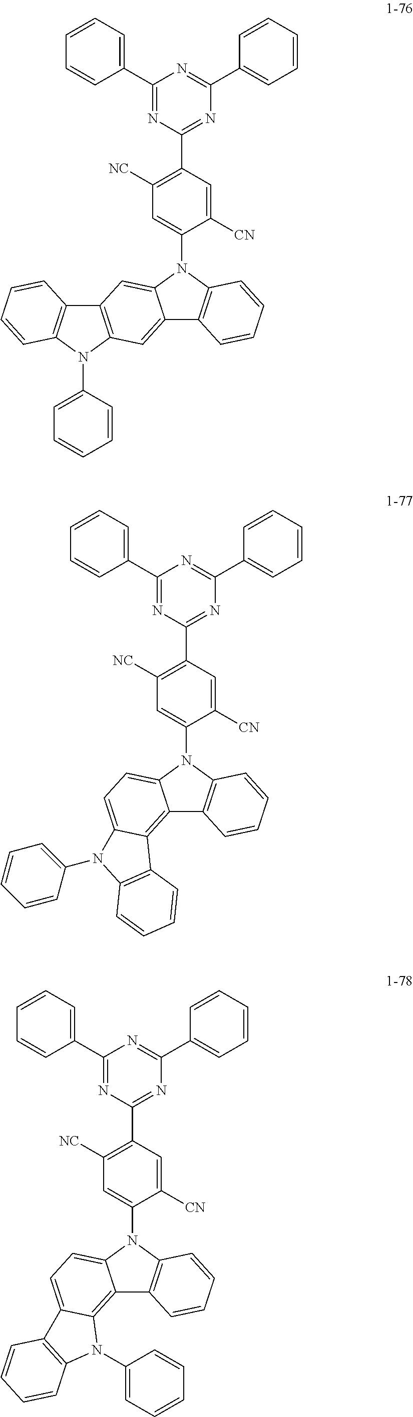 [SMILES: N#Cc1cc(-n2c3ccccc3c3cc4c(cc32)c2ccccc2n4-c2ccccc2)c(C#N)cc1-c1nc(-c2ccccc2)nc(-c2ccccc2)n1 ; N#Cc1cc(-n2c3ccccc3c3c4c5ccccc5n(-c5ccccc5)c4ccc32)c(C#N)cc1-c1nc(-c2ccccc2)nc(-c2ccccc2)n1 ; N#Cc1cc(-n2c3ccccc3c3c2ccc2c4ccccc4n(-c4ccccc4)c23)c(C#N)cc1-c1nc(-c2ccccc2)nc(-c2ccccc2)n1]

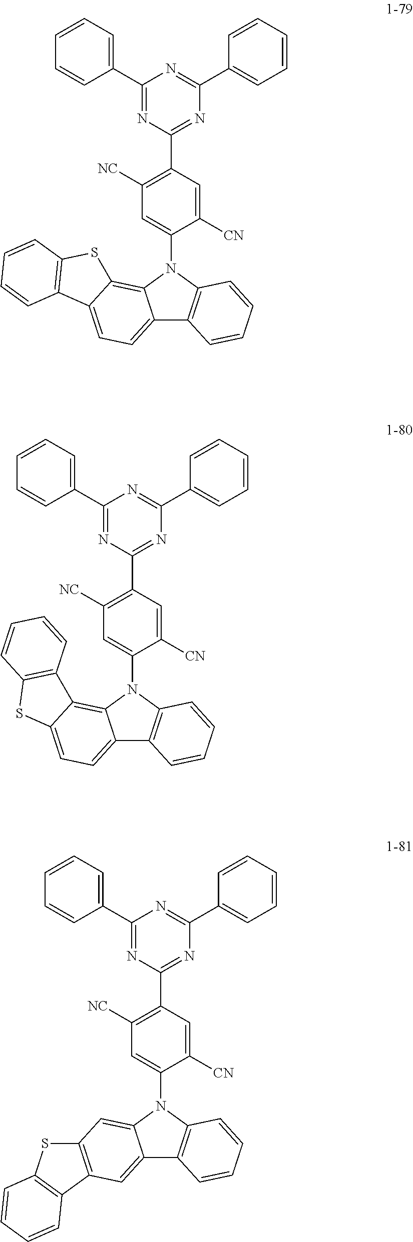 [SMILES: N#Cc1cc(-n2c3ccccc3c3ccc4c5ccccc5sc4c32)c(C#N)cc1-c1nc(-c2ccccc2)nc(-c2ccccc2)n1 ; N#Cc1cc(-n2c3ccccc3c3ccc4sc5ccccc5c4c32)c(C#N)cc1-c1nc(-c2ccccc2)nc(-c2ccccc2)n1 ; N#Cc1cc(-n2c3ccccc3c3cc4c(cc32)sc2ccccc24)c(C#N)cc1-c1nc(-c2ccccc2)nc(-c2ccccc2)n1]

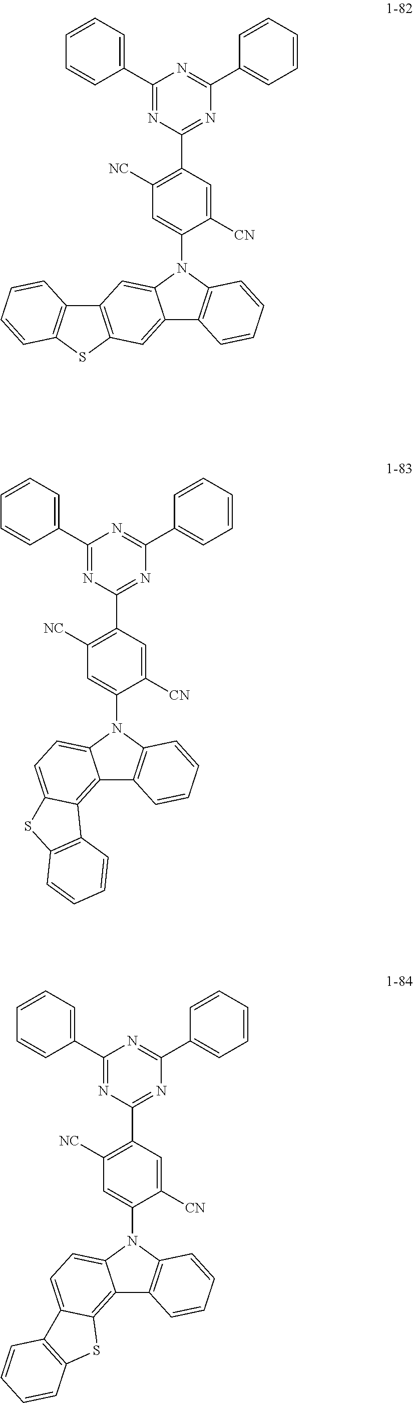 [SMILES: N#Cc1cc(-n2c3ccccc3c3cc4sc5ccccc5c4cc32)c(C#N)cc1-c1nc(-c2ccccc2)nc(-c2ccccc2)n1 ; N#Cc1cc(-n2c3ccccc3c3c4c(ccc32)sc2ccccc24)c(C#N)cc1-c1nc(-c2ccccc2)nc(-c2ccccc2)n1 ; N#Cc1cc(-n2c3ccccc3c3c4sc5ccccc5c4ccc32)c(C#N)cc1-c1nc(-c2ccccc2)nc(-c2ccccc2)n1]

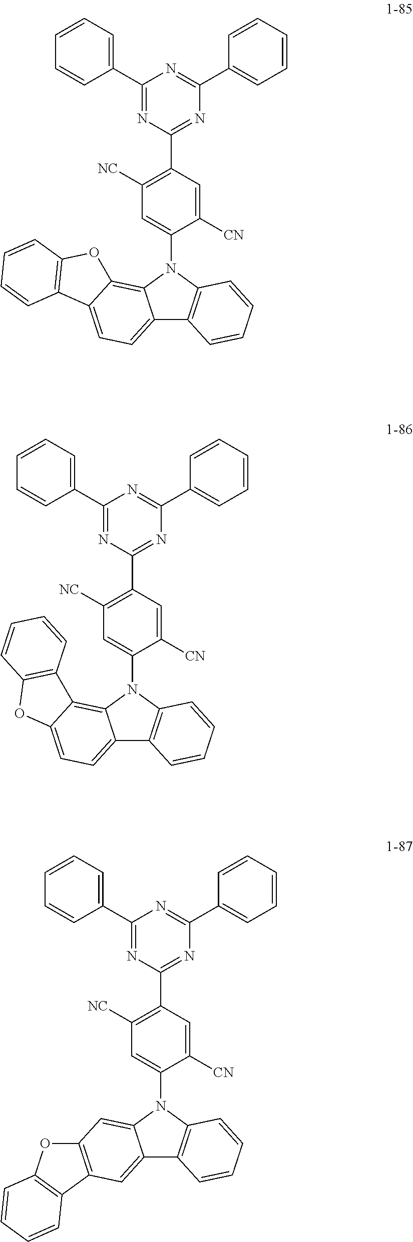 [SMILES: N#Cc1cc(-n2c3ccccc3c3ccc4c5ccccc5oc4c32)c(C#N)cc1-c1nc(-c2ccccc2)nc(-c2ccccc2)n1 ; N#Cc1cc(-n2c3ccccc3c3ccc4oc5ccccc5c4c32)c(C#N)cc1-c1nc(-c2ccccc2)nc(-c2ccccc2)n1 ; N#Cc1cc(-n2c3ccccc3c3cc4c(cc32)oc2ccccc24)c(C#N)cc1-c1nc(-c2ccccc2)nc(-c2ccccc2)n1]

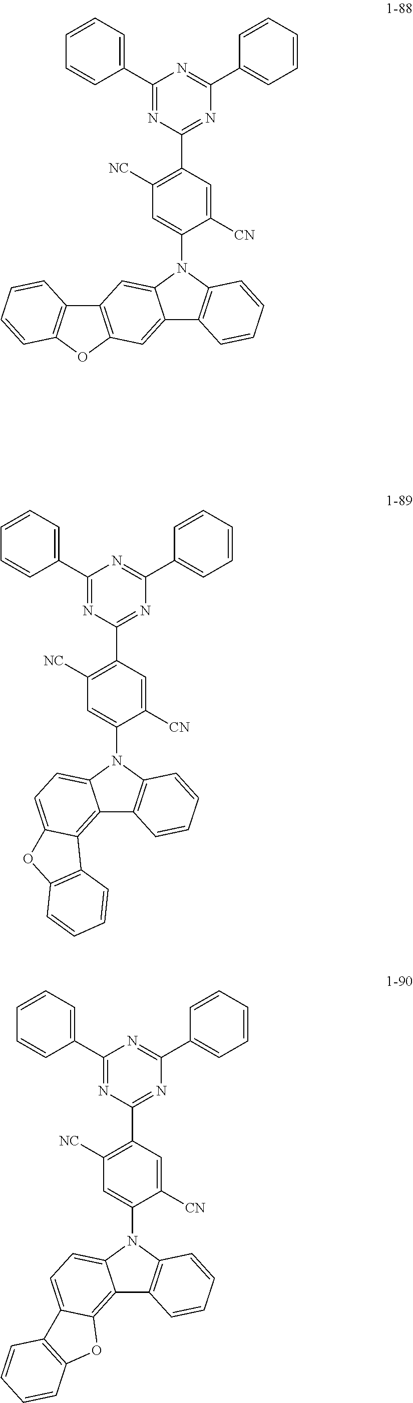 [SMILES: N#Cc1cc(-n2c3ccccc3c3cc4oc5ccccc5c4cc32)c(C#N)cc1-c1nc(-c2ccccc2)nc(-c2ccccc2)n1 ; N#Cc1cc(-n2c3ccccc3c3c4c(ccc32)oc2ccccc24)c(C#N)cc1-c1nc(-c2ccccc2)nc(-c2ccccc2)n1 ; N#Cc1cc(-n2c3ccccc3c3c4oc5ccccc5c4ccc32)c(C#N)cc1-c1nc(-c2ccccc2)nc(-c2ccccc2)n1]

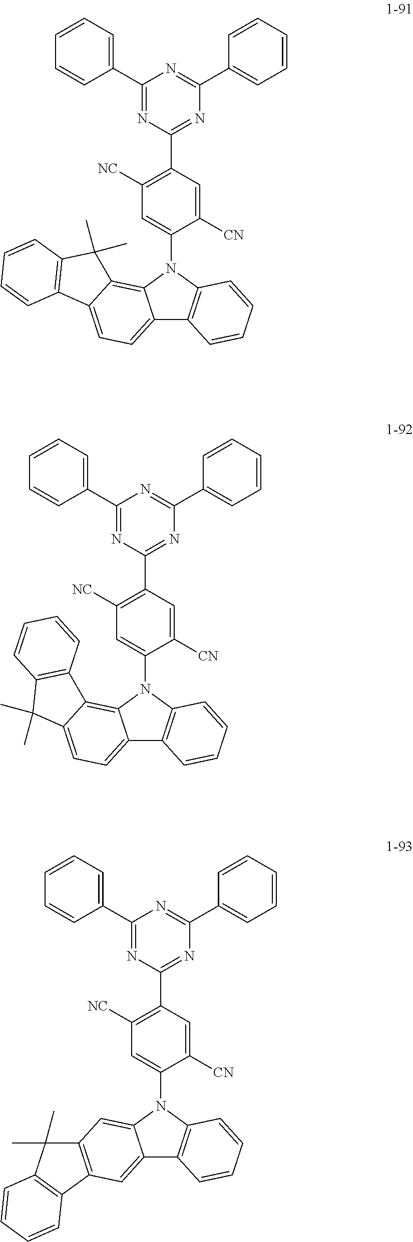 [SMILES: CC1(C)c2ccccc2-c2ccc3c4ccccc4n(-c4cc(C#N)c(-c5nc(-c6ccccc6)nc(-c6ccccc6)n5)cc4C#N)c3c21 ; CC1(C)c2ccccc2-c2c1ccc1c3ccccc3n(-c3cc(C#N)c(-c4nc(-c5ccccc5)nc(-c5ccccc5)n4)cc3C#N)c21 ; CC1(C)c2ccccc2-c2cc3c4ccccc4n(-c4cc(C#N)c(-c5nc(-c6ccccc6)nc(-c6ccccc6)n5)cc4C#N)c3cc21]

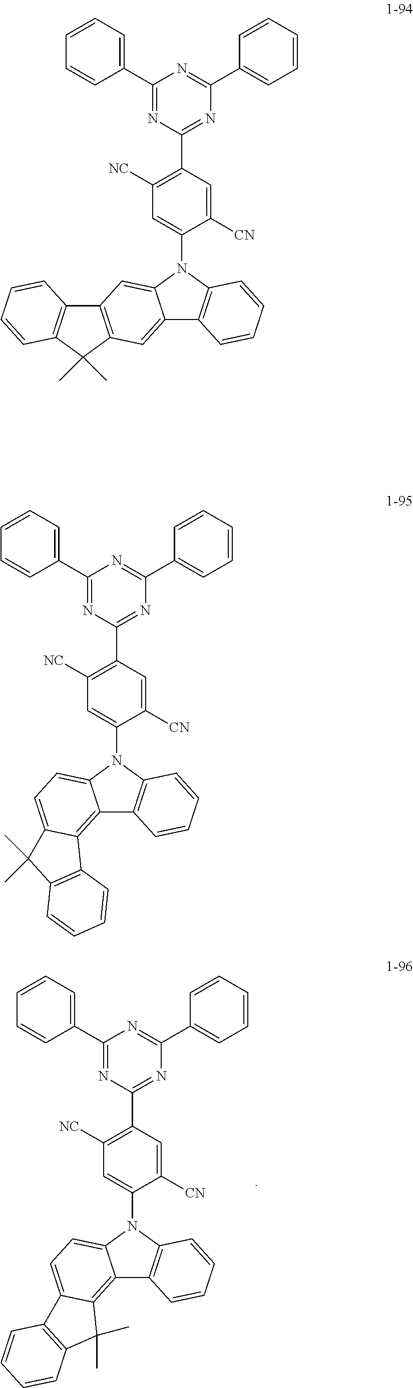 [SMILES: CC1(C)c2ccccc2-c2cc3c(cc21)c1ccccc1n3-c1cc(C#N)c(-c2nc(-c3ccccc3)nc(-c3ccccc3)n2)cc1C#N ; CC1(C)c2ccccc2-c2c1ccc1c2c2ccccc2n1-c1cc(C#N)c(-c2nc(-c3ccccc3)nc(-c3ccccc3)n2)cc1C#N ; CC1(C)c2ccccc2-c2ccc3c(c21)c1ccccc1n3-c1cc(C#N)c(-c2nc(-c3ccccc3)nc(-c3ccccc3)n2)cc1C#N]

9. The organic light emitting diode of claim 1, wherein the second compound is selected from the following compounds having the structure of Formula 5:

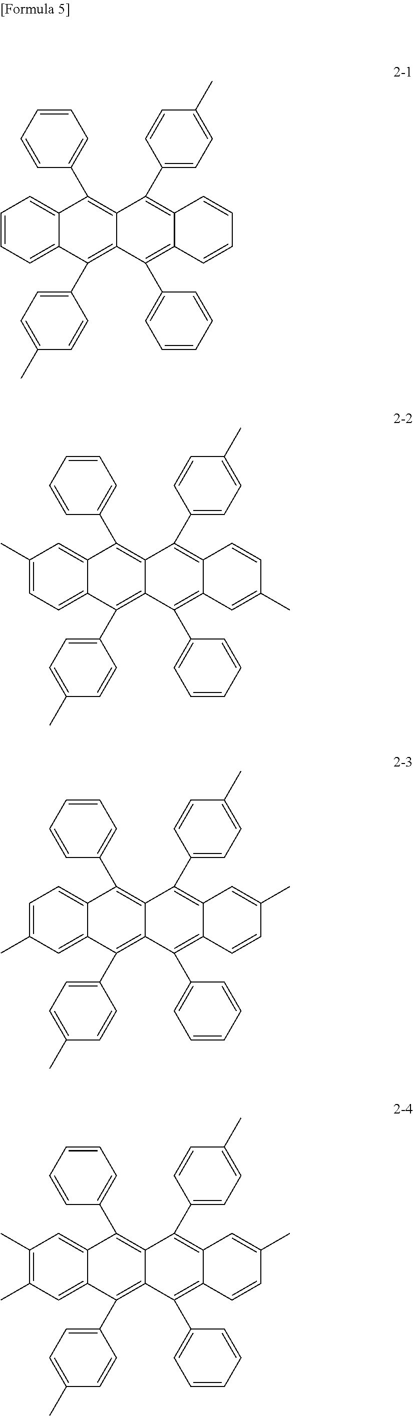

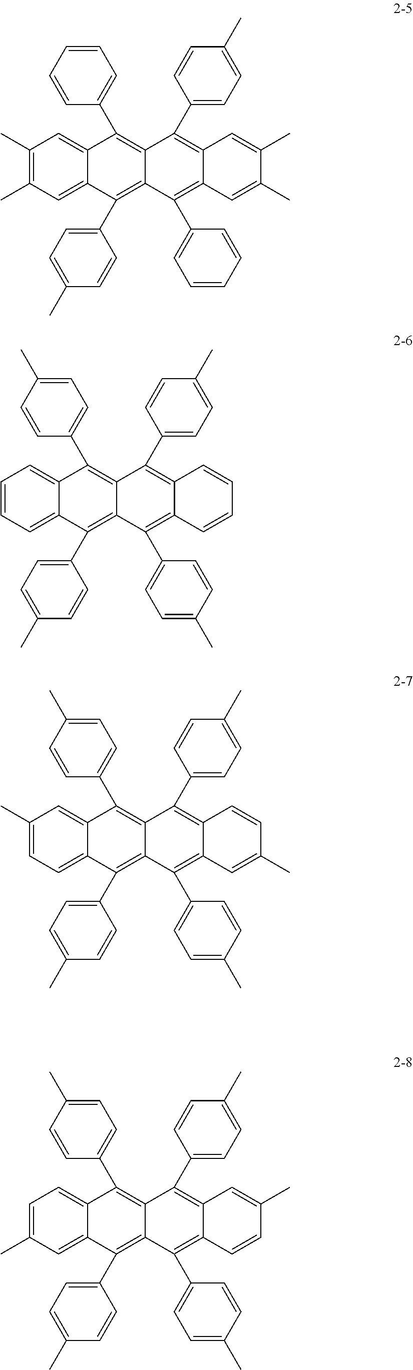

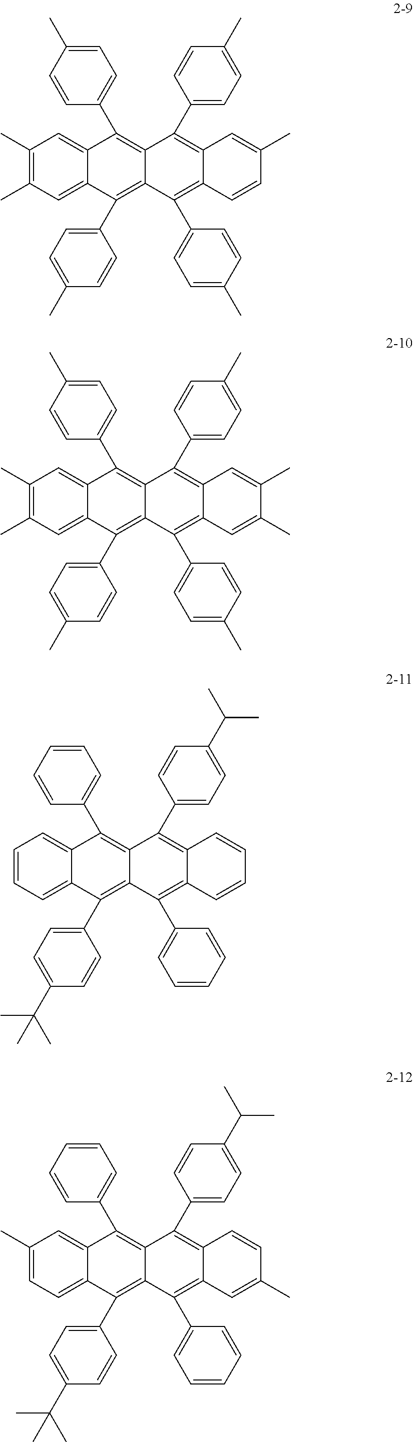

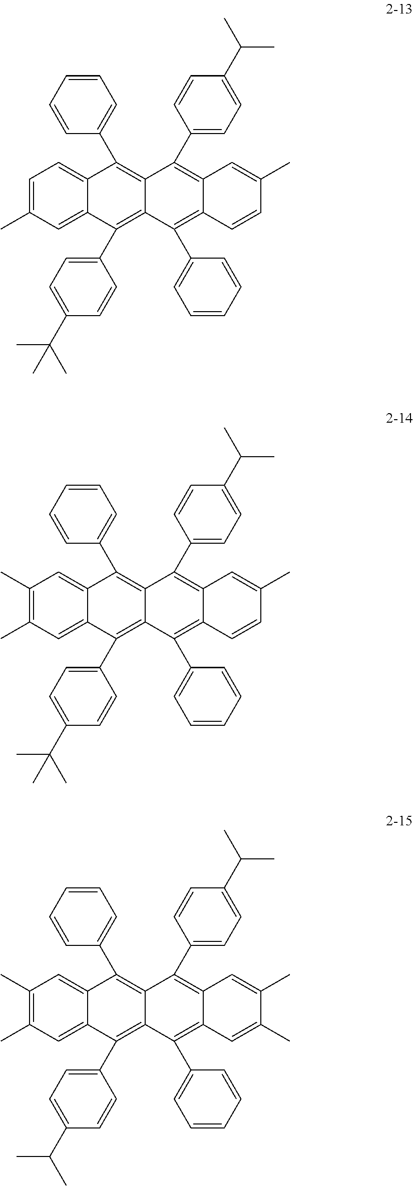

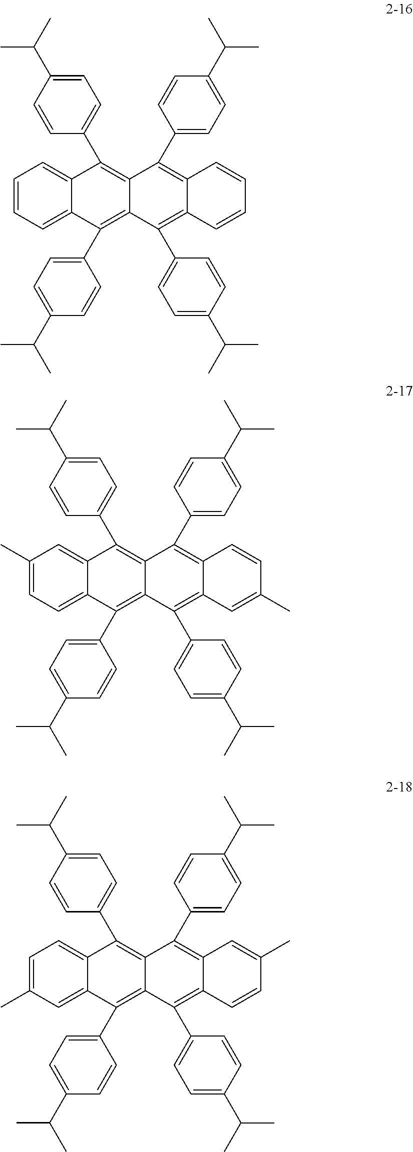

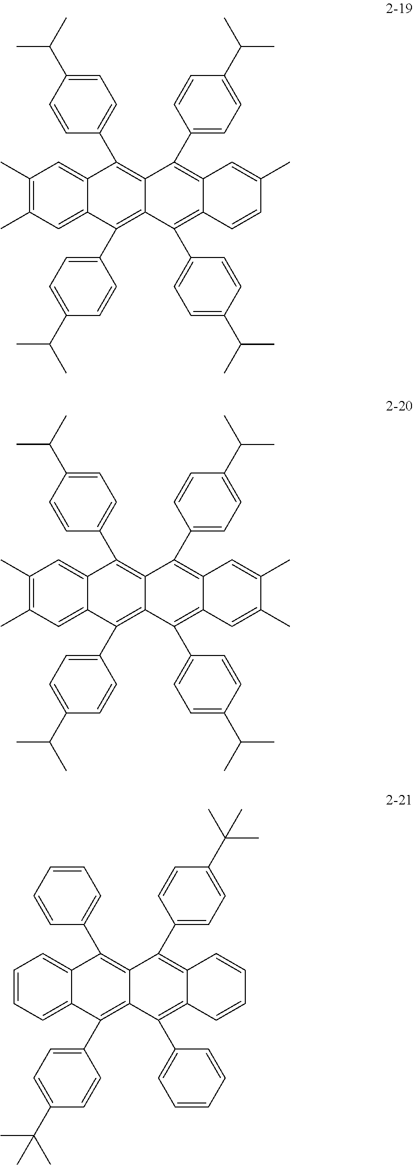

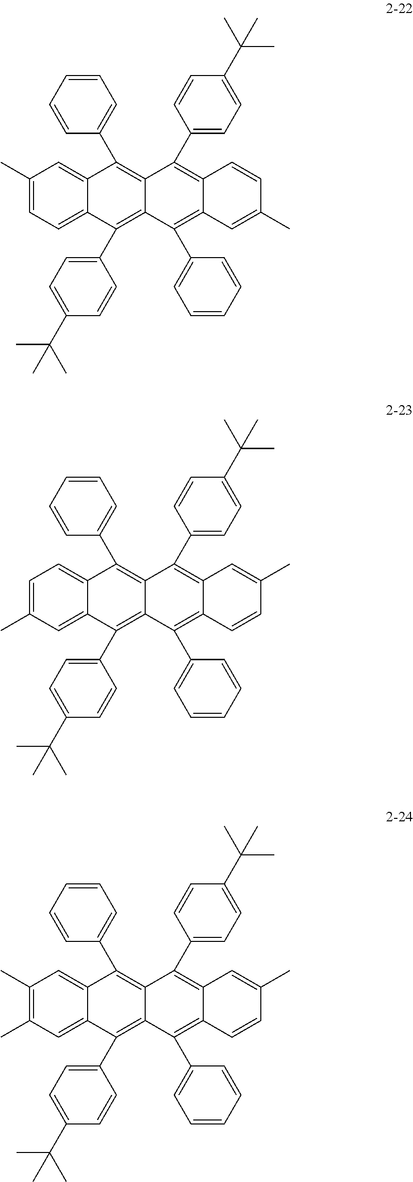

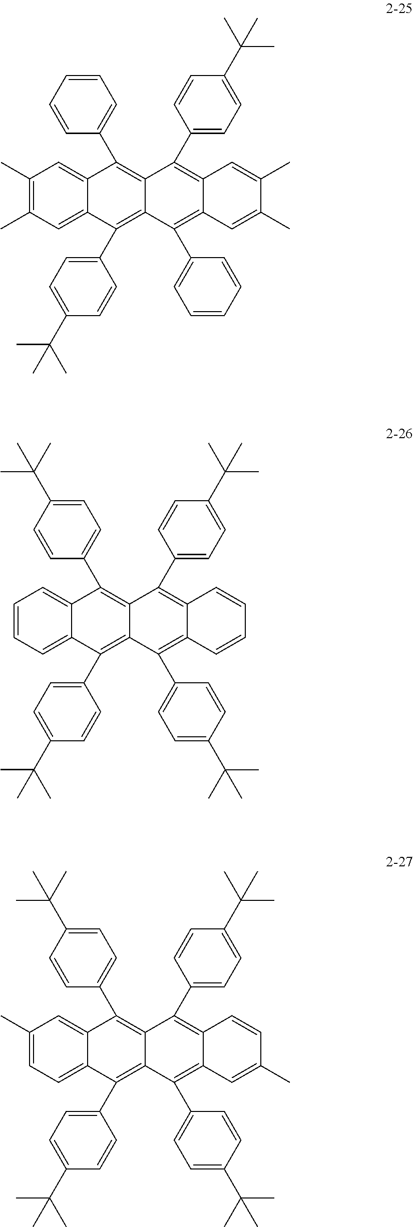

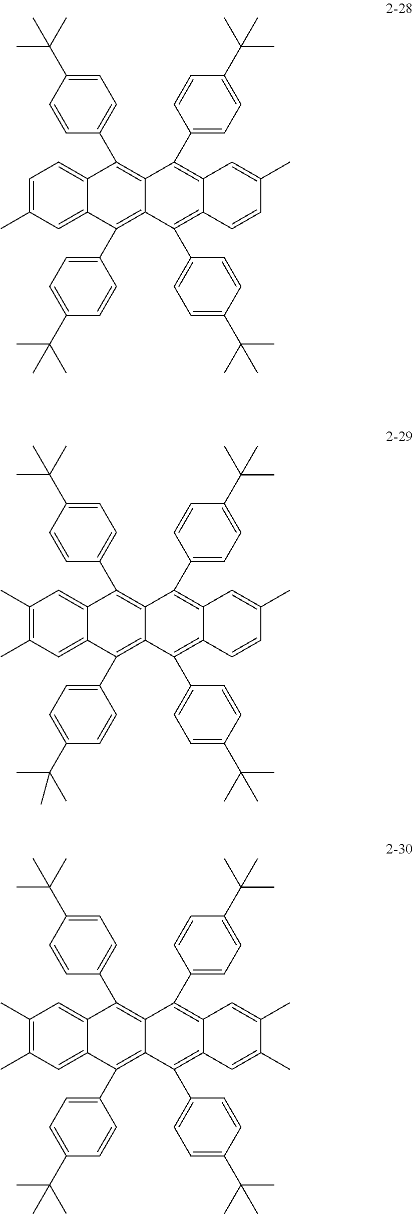

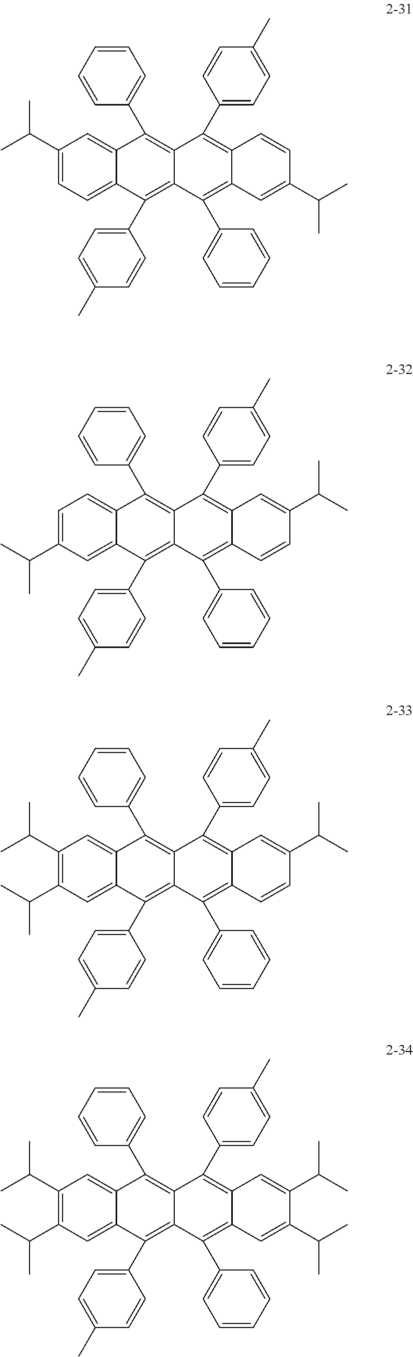

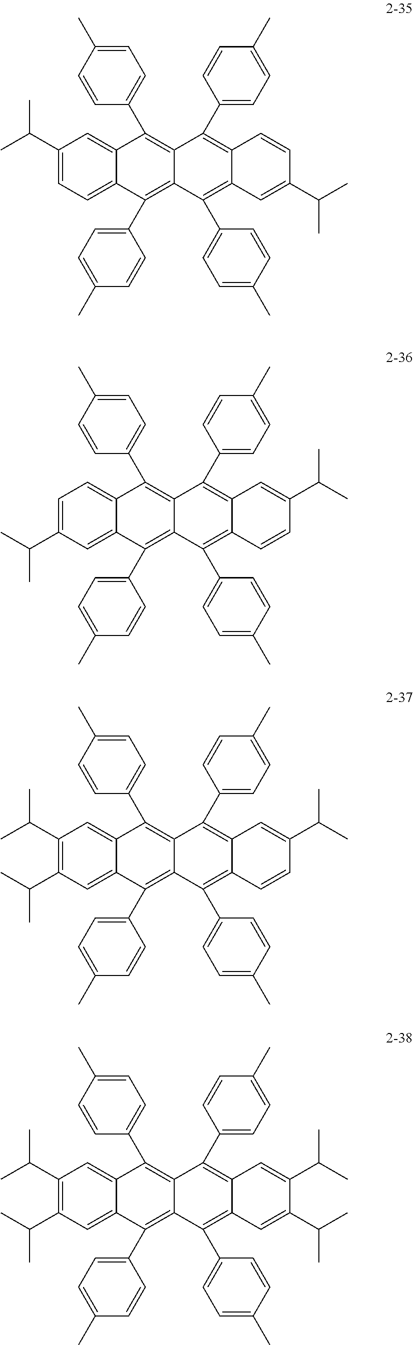

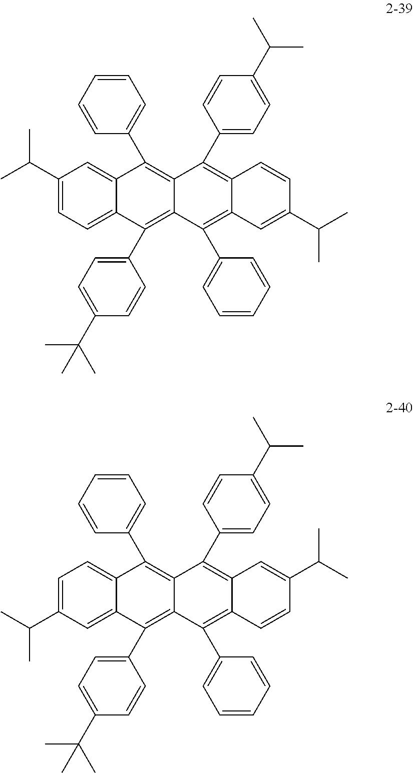

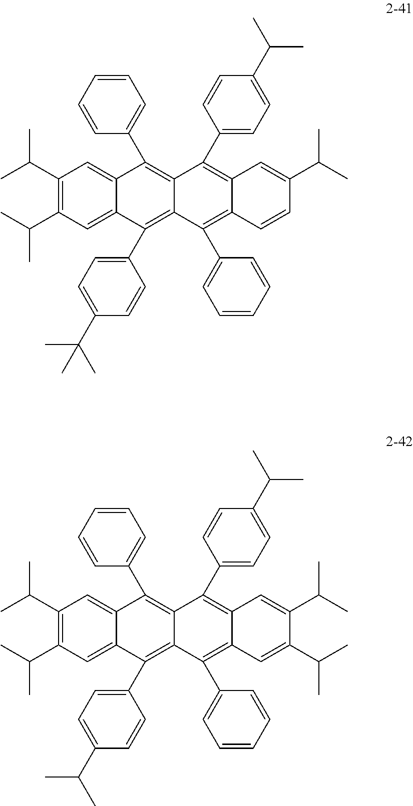

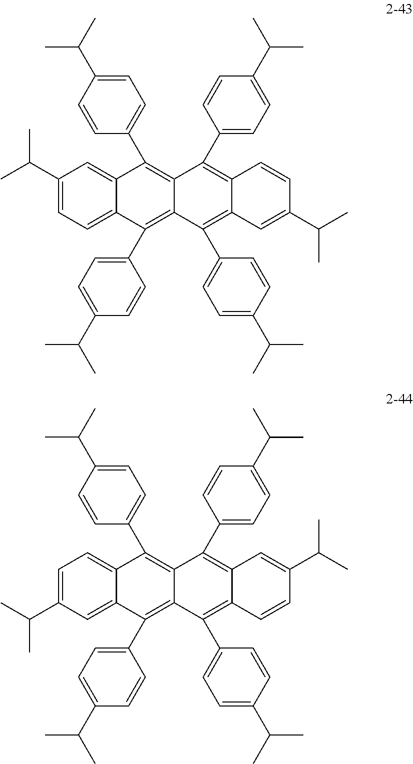

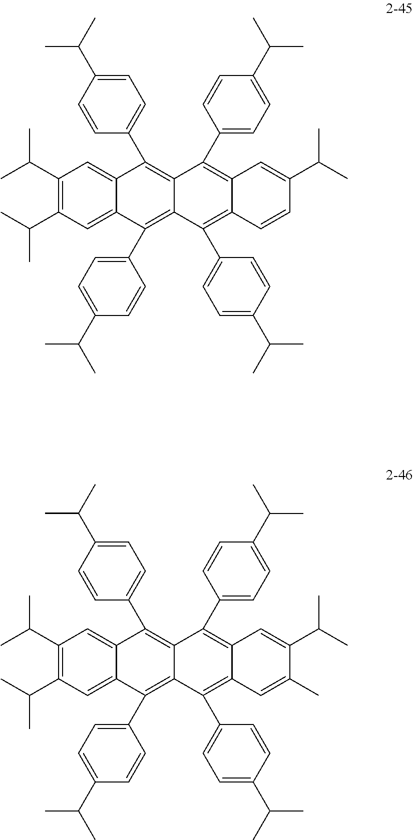

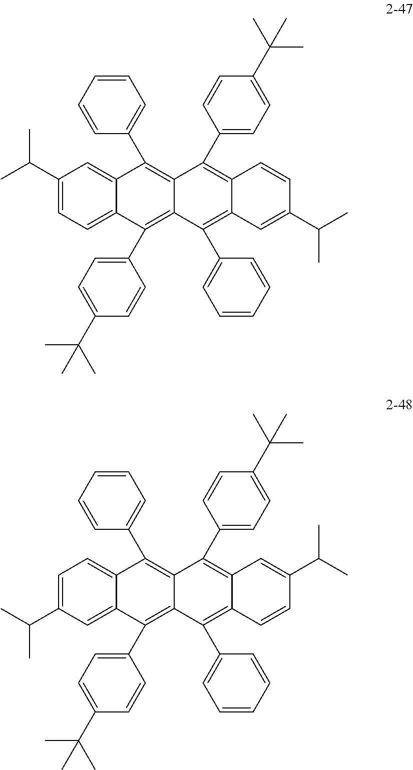

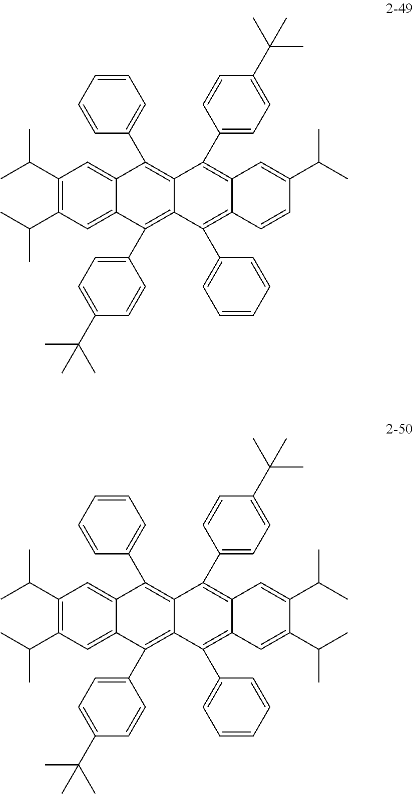

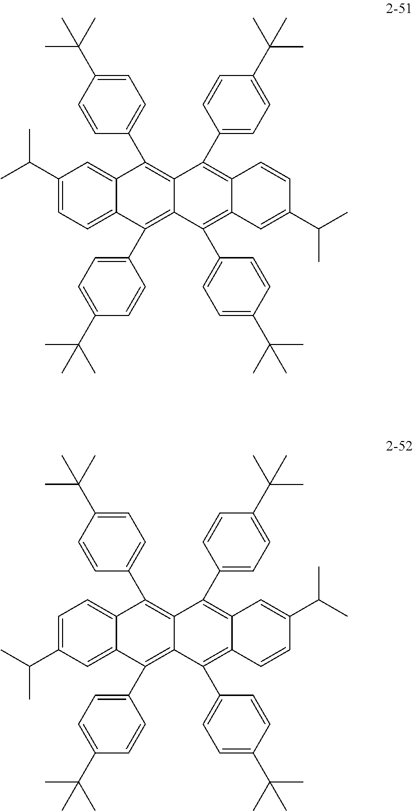

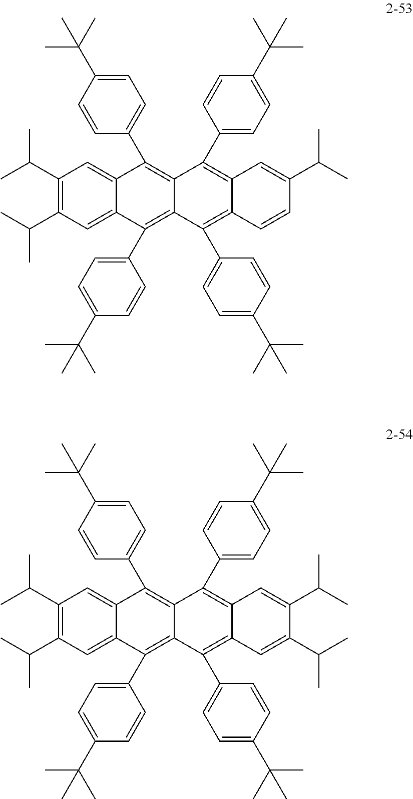

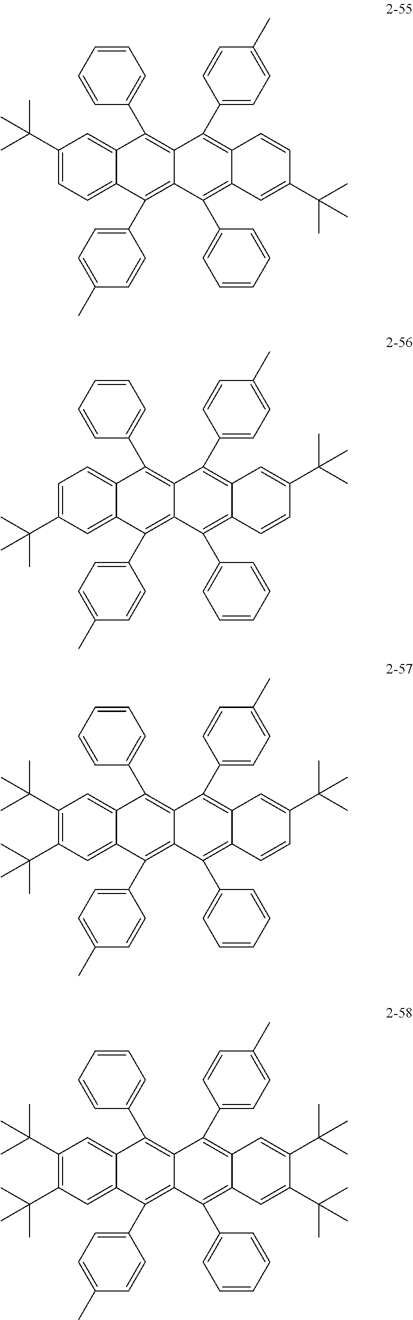

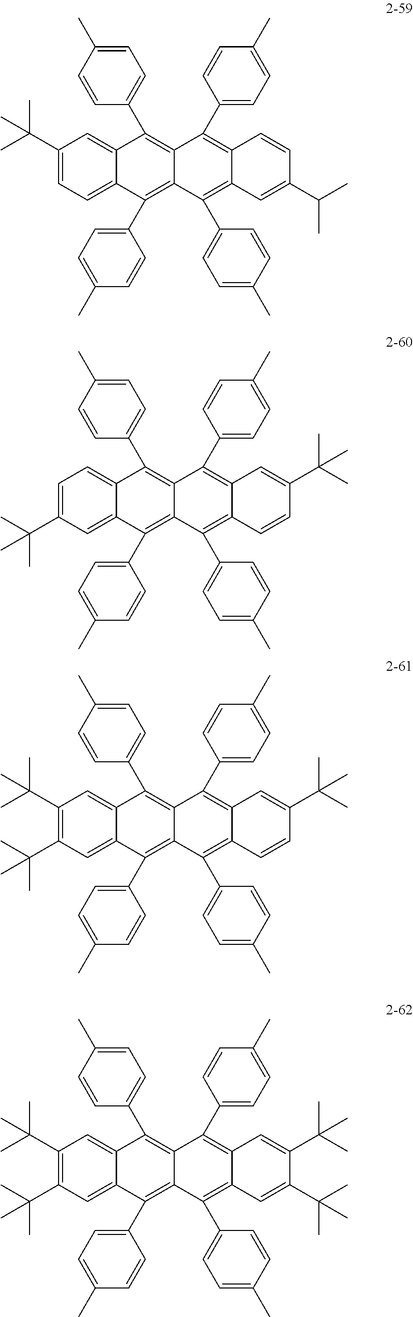

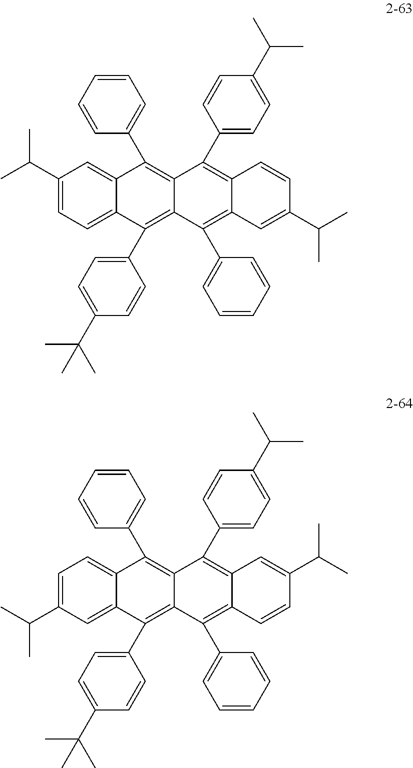

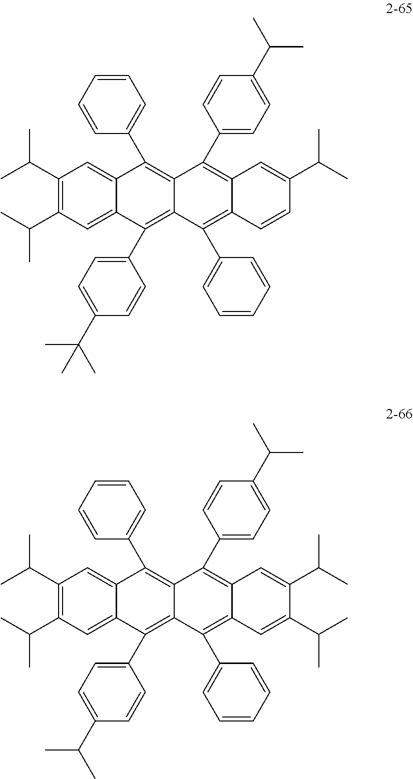

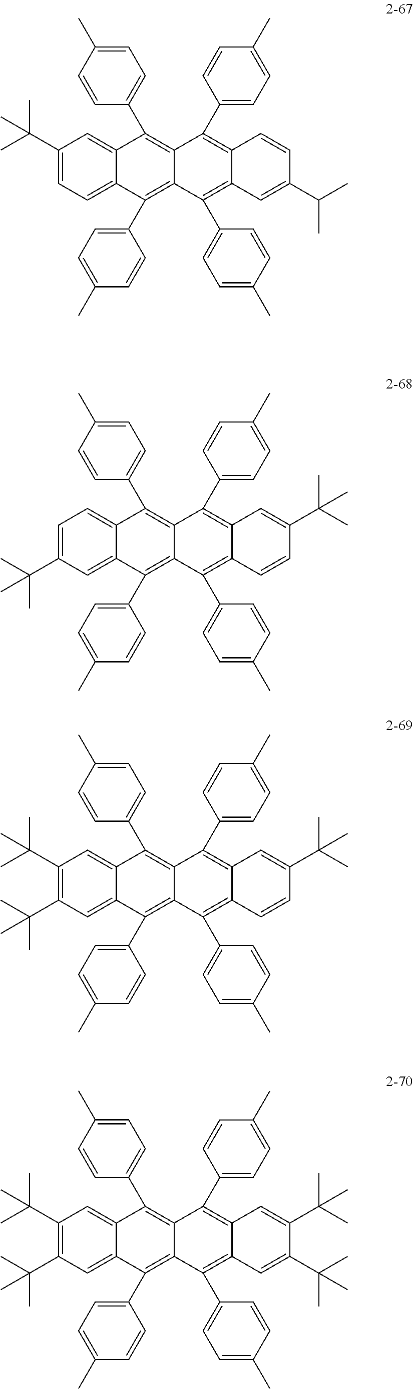

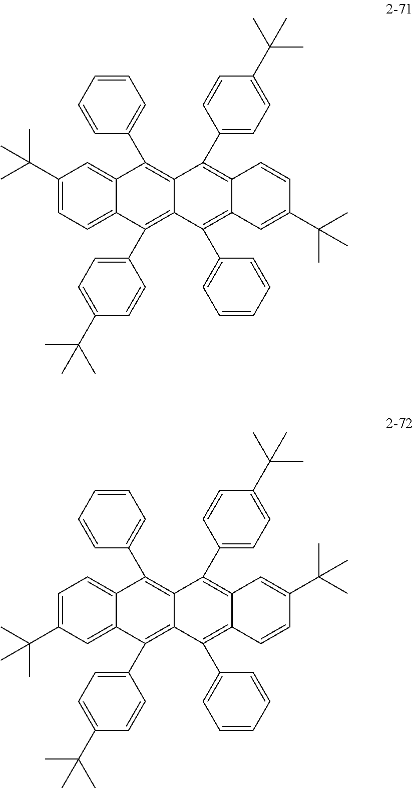

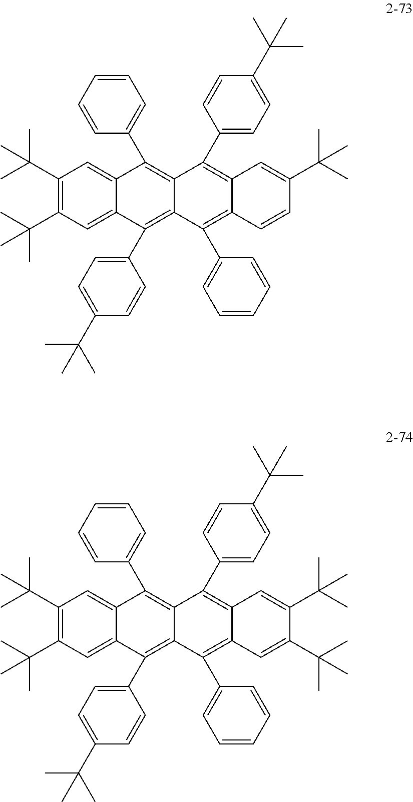

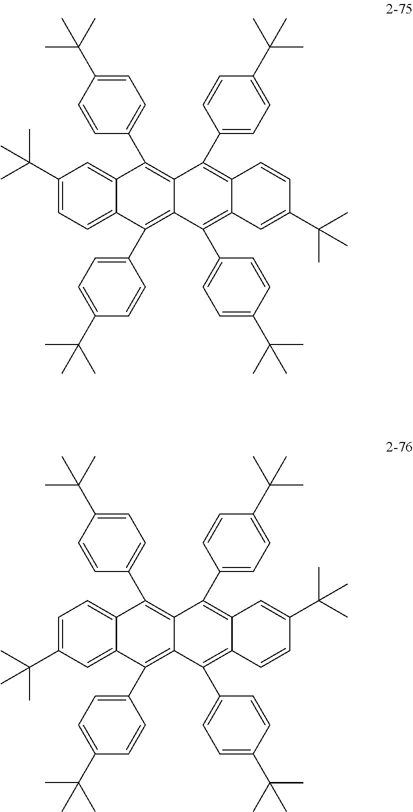

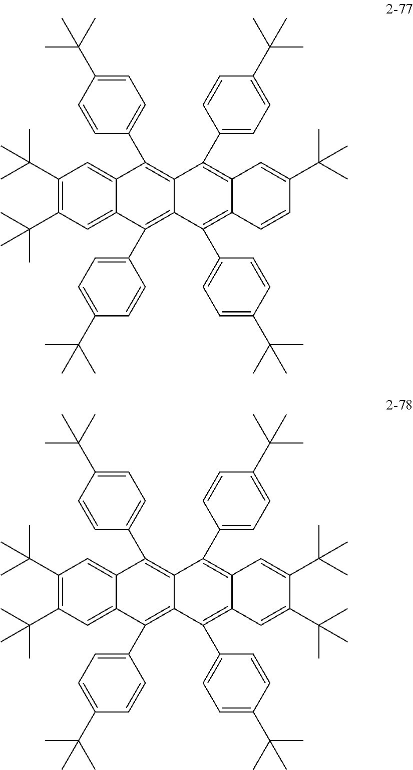

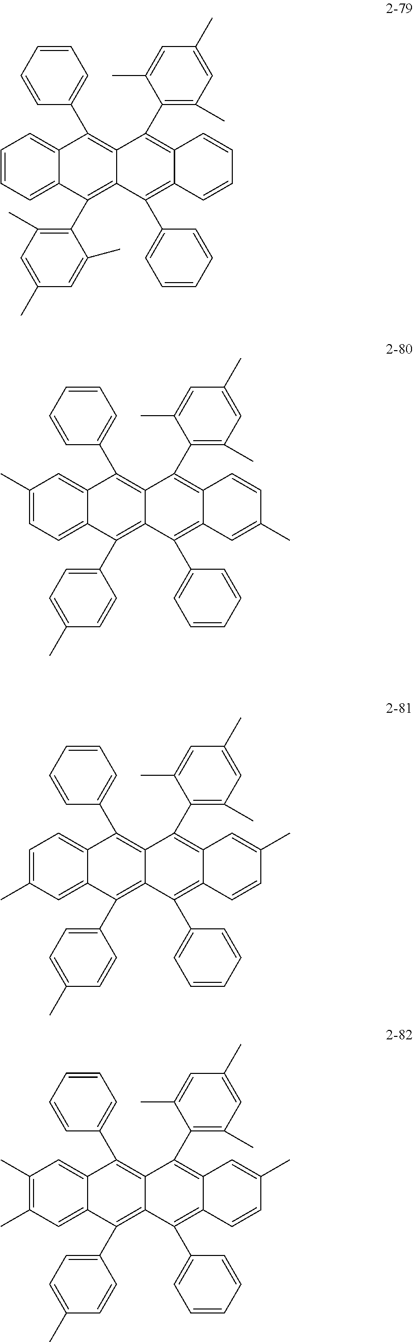

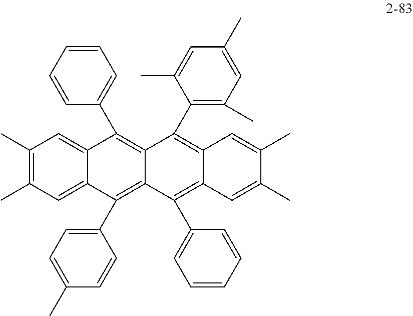

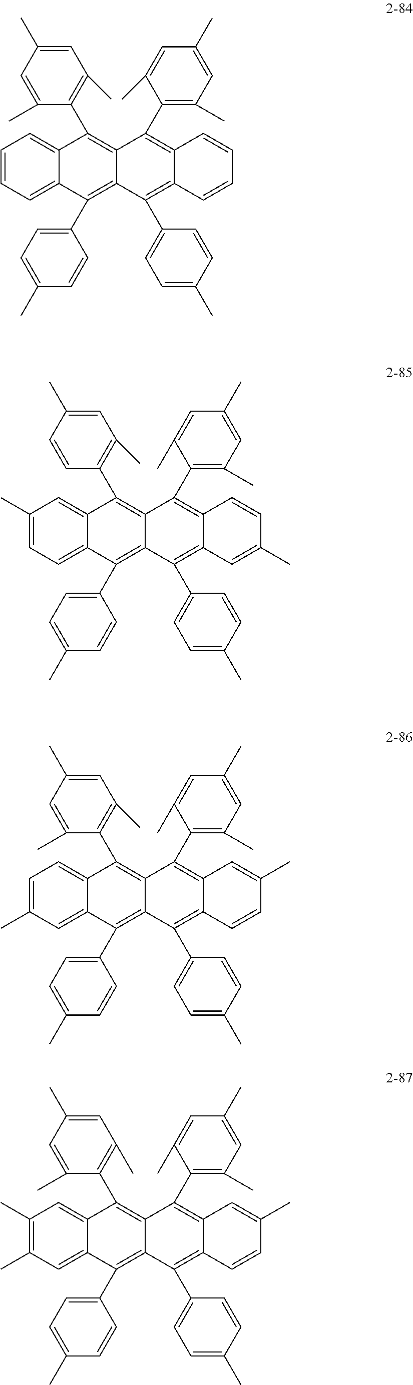

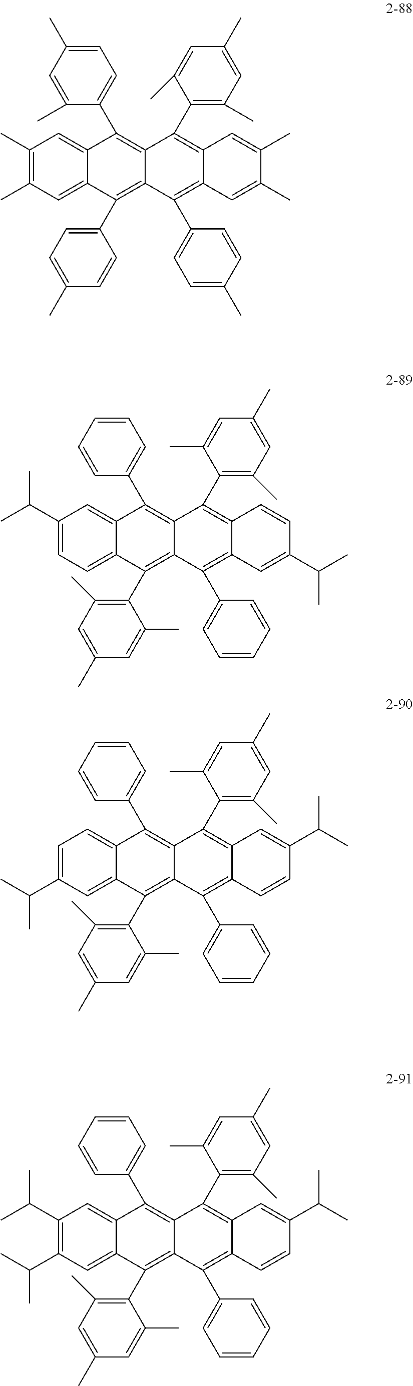

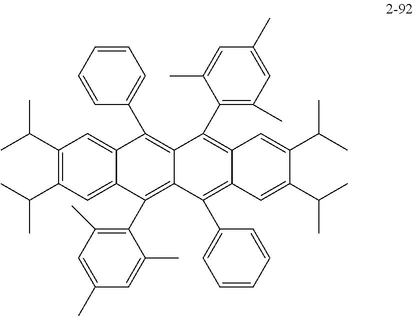 [SMILES: Cc1cc(C)c(-c2c3cc(C(C)C)c(C(C)C)cc3c(-c3ccccc3)c3c(-c4c(C)cc(C)cc4C)c4cc(C(C)C)c(C(C)C)cc4c(-c4ccccc4)c23)c(C)c1]

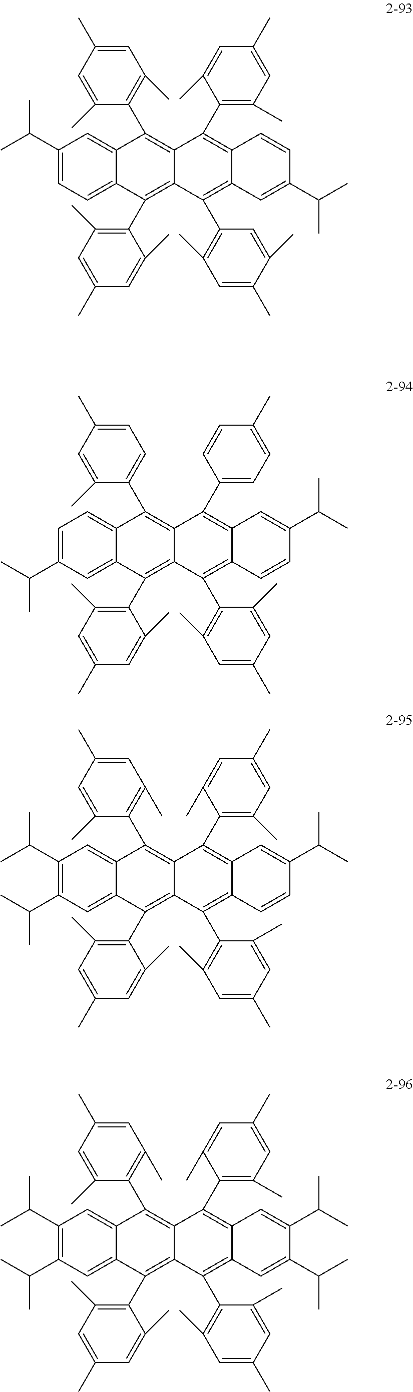

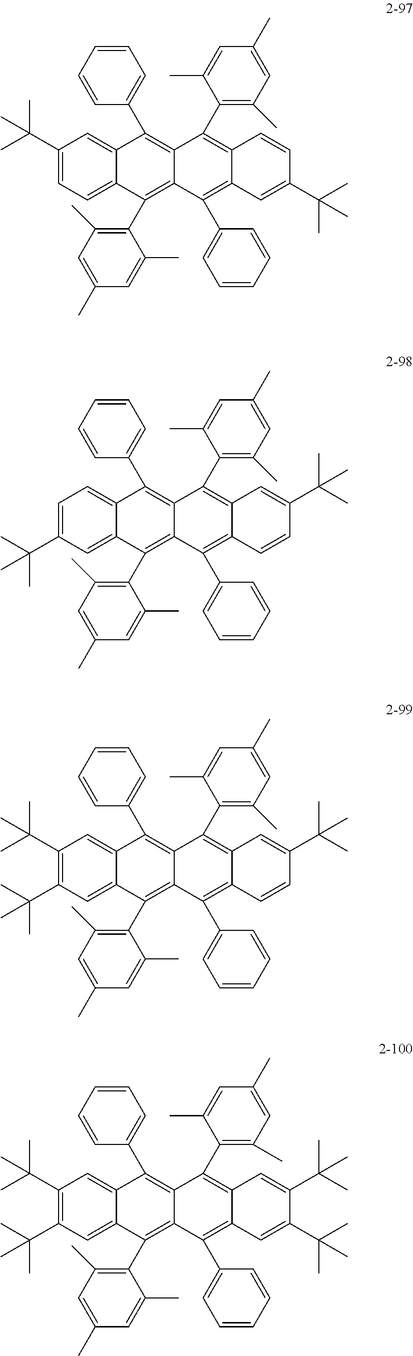

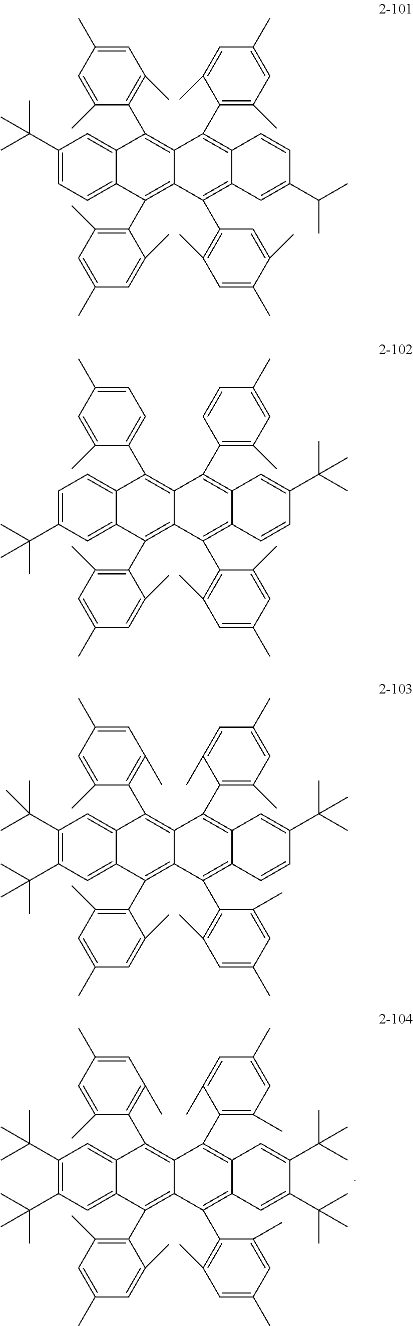

10. The organic light emitting diode of claim 1, wherein contents of the first compound is larger than contents of the second compound in the at least one emitting material layer.

11. The organic light emitting diode of claim 1, wherein the at least one emitting material layer has a mono-layered structure.

12. The organic light emitting diode of claim 1, the at least one emitting material layer further comprises a third compound.

13. The organic light emitting diode of claim 12, wherein a singlet energy level of the third compound is higher than a singlet energy level of the first compound.

14. The organic light emitting diode of claim 1, wherein the at least one emitting material layer includes a first emitting material layer disposed between the first and second electrodes and a second emitting material layer disposed between the first electrode and the first emitting material layer or between the first emitting material layer and the second electrode.

15. The organic light emitting diode of claim 14, wherein the first emitting material layer comprises the first compound and the second emitting material layer comprises the second compound.

16. The organic light emitting diode of claim 14, the at least one emitting material layer further comprises a third emitting material layer disposed oppositely to the second emitting material layer with respect to the first emitting material layer.

17. The organic light emitting diode of claim 16, wherein the first emitting material layer comprises the first compound and each of the second and third emitting material layers includes the second compound.

18. An organic light emitting device comprising:
a substrate; and
an organic light emitting diode of claim 1 over the substrate.

19. The organic light emitting device of claim 18, further comprises a thin film transistor over the substrate and connected to the organic light emitting diode.

20. The organic light emitting device of claim 18, further comprises a color filter layer disposed over the organic light emitting diode or disposed between the substrate and the organic light emitting diode.
